(19) United States
(12) Patent Application Publication
(10) Pub. No.: US 2010/0221349 A1
(43) Pub. Date: Sep. 2, 2010

(54) NUCLEIC ACID CONSTRUCTS

(75) Inventor: James Fuller, Cranberry TWP, PA (US)

Correspondence Address: FOLEY AND LARDNER LLP;SUITE 500, 3000 K STREET NW, WASHINGTON, DC, 20007, US

(73) Assignees: Powderject Vaccines, Inc.; Powdermed Limited

(21) Appl. No.: 11/815,278

(22) PCT Filed: Feb. 1, 2006
(86) PCT No.: PCT/GB2006/000344

§ 371 Date: Sep. 18, 2007

Related U.S. Application Data
(60) Provisional application No. 60/648,382, filed on Feb. 1, 2005.
(60) Provisional application No. 60/672,497, filed on Apr. 19, 2005.

(30) Foreign Application Priority Data
Apr. 20, 2005 (GB) 0507997.5

(51) Int. Cl. A61K 39/145; C12N 15/44; C12N 15/52; C12N 15/63; A61K 9/14; A61K 31/7052; C12N 15/00; A61P 31/16; A61M 5/30

(52) U.S. Cl. 424/490; 536/23.72; 536/23.2; 435/320.1; 514/44 R; 424/206.1; 435/455; 604/68

(57) ABSTRACT

A nucleic acid construct comprising a chimeric promoter sequence and a cloning site for insertion of a coding sequence in operable linkage with the chimeric promoter, wherein the chimeric promoter sequence comprises: (a) a Hcmv immediate early promoter sequence; (b) exon 1 and at least a part of exon 2 of the hCMV major immediate early gene; and (c) a heterologous intron provided in place of the intron A region of the hCMV major immediate early gene.

### FIELD OF THE INVENTION

The invention relates to the fields of molecular biology and immunology and generally to reagents useful in nucleic acid immunisation techniques. More specifically, the invention relates to nucleic acid constructs for the expression of polypeptides and in particular antigenic polypeptides, particularly influenza antigens as well as the expression of adjuvant polypeptides and to nucleic acid immunisation strategies using such reagents

### BACKGROUND OF THE INVENTION

Gene therapy and nucleic acid immunisation are promising approaches for the treatment and prevention of both acquired and inherited diseases. These techniques provide for the transfer of a desired nucleic acid into a subject with subsequent in vivo expression. Transfer can be accomplished by transfecting the subject's cells or tissues ex vivo and reintroducing the transformed material into the host. Alternatively, the nucleic acid can be administered in vivo directly to the recipient.

Each of these techniques requires efficient expression of the nucleic acid in the transfected cell, to provide a sufficient amount of the therapeutic or antigenic gene product. Several factors are known to affect the levels of expression obtained, including transfection efficiency, and the efficiency with which the gene or sequence of interest is transcribed and the mRNA translated.

A number of expression systems have been described in the art, each of which typically consists of a vector containing a gene or nucleotide sequence of interest operably linked to expression control sequences. These control sequences include transcriptional promoter sequences and transcriptional start and termination sequences. Commonly used promoters for mammalian cell expression systems include the SV40 early promoter, a cytomegalovirus (CMV) promoter such as the CMV immediate early promoter (Chapman et al (1991) Nucl. Acids Res. 19:3979-3986), the mouse mammary tumour virus long terminal repeat (LTR) promoter, the adenovirus major late promoter (Ad MLP) and the herpes simplex virus (HSV) promoter, among others. Nonviral promoters, such as a promoter derived from the murine metallothionein gene are also commonly used.

Expression systems often include transcriptional modulator elements, referred to as “enhancers”. Enhancers are broadly defined as a cis-acting agent, which when operably linked to a promoter/gene sequence, will increase transcription of that gene sequence. Enhancers can function from positions that are much further away from a sequence of interest than other expression control elements (e.g. promoters), and can operate when positioned in either orientation relative to the sequence of interest (Banerji et al. (1981) Cell 27:299-308, deVilleirs et al. (1981) Nucl. Acids Res 9: 6251-6264). Enhancers have been identified from a number of viral sources, including polyoma virus, BK virus, cytomegalovirus (CMV), adenovirus, simian virus 40 (SV40), Moloney sarcoma virus, bovine papilloma virus and Rous sarcoma virus (deVilleirs et al supra, Rosenthal et al. (1983) Science 222:749-755, Hearing et al. (1983) Cell 33:695-703, Weeks et al. (1983) Mol. Cell. Biol. 3:1222-1234, Levinson et al. (1982) Nature 295: 568-572, and Luciw et al. (1983) Cell 33: 705-716).

A number of expression systems for nucleic acid immunisation and gene therapy make use of the hCMV immediate early promoter. See eg U.S. Pat. Nos. 5,168,062 and 5,385,839 to Stinski, and EP Patent Specification 0323997 B1. Expression vectors using the hCMV immediate early promoter include for example, pWRG7128 (Roy et al, Vaccine 19, 764-778, 2001), and pBC12/CMV and pJW4303 which are mentioned in WO 95/20660. Chapman et al (1991) supra report reduced levels of expression from the hCMV immediate early promoter in the absence of hCMV Intron A.

### SUMMARY OF THE INVENTION

A nucleic acid construct has been developed using manipulated viral promoter/expression sequences that provides enhanced expression of heterologous coding sequences in host cells. The construct is suitable for efficient expression of genes and in particular antigen-encoding genes, and can therefore be used in nucleic acid immunisation. The construct may also be used to express adjuvant polypeptides The construct can be provided on carrier particles, for use in particle-mediated nucleic acid immunisation. In an especially preferred embodiment of the invention the construct encodes an influenza antigen, immunogenic fragment thereof or an immunogenic variant of either. In a further especially preferred embodiment the construct encodes an adjuvant polypeptide.

Accordingly, the present invention provides a nucleic acid construct suitable for delivery to a subject for inducing an immune response against influenza virus hemagglutinin (HA) antigen, which construct comprises:
(i) a chimeric promoter sequence comprising:
   (a) a hCMV immediate early promoter sequence;
   (b) exon 1 and at least a part of exon 2 of the hCMV major immediate early gene; and
   (c) a heterologous intron provided in place of the intron A region of the hCMV major immediate early gene;
(ii) a coding sequence in operable linkage with the chimeric promoter, where the coding sequence encodes an influenza virus hemagglutinin (HA) antigen, an immunogenic fragment thereof or an immunogenic variant of said antigen or fragment having at least 80% amino acid homology to said antigen or fragment;
(iii) a non-translated leader sequence which is derived from HBVpreS2 antigen sequence, HBV e-antigen sequence or HSV type 2gD antigen sequence and which is in operable linkage with the chimeric promoter; and
(iv) an enhancer sequence which is derived from a 3′ untranslated region (UTR) of a HBsAg sequence or of a simian CMV immediate early gene sequence, which is in operable linkage with the chimeric promoter and which is downstream of coding sequence.

The present invention also provides a nucleic acid construct suitable for delivery to a subject for inducing an immune response against influenza virus hemagglutinin (HA) antigen, which construct comprises:
(i) a chimeric promoter sequence comprising:
   (a) a hCMV immediate early promoter sequence;
   (b) exon 1 and at least a part of exon 2 of the hCMV major immediate early gene; and
   (c) a heterologous intron provided in place of the intron A region of the hCMV major immediate early gene; and
(ii) a coding sequence in operable linkage with the chimeric promoter, where the coding sequence encodes an influenza virus hemagglutinin (HA) antigen, an immunogenic fragment thereof or an immunogenic variant of said antigen or fragment having at least 80% amino acid homology to said antigen or fragment.

Further, the present invention provides a nucleic acid construct comprising a chimeric promoter sequence and a cloning site for insertion of a coding sequence in operable linkage with the chimeric promoter, wherein the chimeric promoter sequence comprises:
(a) a hCMV immediate early promoter sequence;
(b) exon 1 and at least a part of exon 2 of the hCMV major immediate early gene; and
(c) a heterologous intron provided in place of the intron A region of the hCMV major immediate early gene.

In a preferred instance, the construct comprises a coding sequence inserted into the cloning site. Thus, a coding sequence may be provided in the cloning site.

In a further preferred embodiment a construct of the invention may further comprise:
(a) a non-translated leader sequence which is derived from HBVpreS2 antigen sequence, HBV e-antigen sequence or HSV type 2 gD antigen sequence and which is in operable linkage with the chimeric promoter; and/or
(b) an enhancer sequence which is derived from a 3′ untranslated region (UTR) of a HBsAg sequence, or a 3′ UTR of a simian CMV immediate early gene sequence and which is in operable linkage with the chimeric promoter, wherein the enhancer sequence is downstream of the cloning site.

The invention also provides a nucleic acid construct comprising a promoter sequence and a coding sequence operably linked to the promoter, where the construct further comprises:
(a) a non-translated leader sequence which is derived from HBVpreS2 antigen sequence, HBV e-antigen sequence or HSV type 2gD antigen sequence, which is in operable linkage with the coding sequence and promoter which is heterologous to the coding sequence; and/or
(b) an enhancer sequence 3′ of and operably linked to the coding sequence, where the enhancer sequence is derived from a 3′ UTR of an HBsAg sequence or a 3′ UTR of a simian CMV immediate early gene sequence, and the coding sequence is heterologous to the 3′ enhancer sequence.

The invention also provides a nucleic acid construct comprising:
(i) a chimeric promoter sequence which comprises:
   (a) a hCMV immediate early promoter sequence;
   (b) exon 1 and at least a part of exon 2 of the hCMV major immediate early gene; and
   (c) a heterologous intron provided in place of the intron A region of the hCMV major immediate early gene; and
(ii) a cloning site for insertion of a coding sequence in operable linkage with the chimeric promoter; and
(iii) (a) a non-translated leader sequence which is derived from HBV preS2 antigen sequence, HBV e-antigen sequence or HSV type 2gD antigen sequence and which is in operable linkage with the chimeric promoter; and/or
   (b) an enhancer sequence which is derived from a 3′ untranslated region (UTR) of a HBsAg sequence, or a 3′ UTR of a simian CMV immediate early gene sequence and which is in operable linkage with the chimeric promoter, wherein the enhancer sequence is downstream of the cloning site.

The invention also provides a nucleic acid construct comprising:
(i) a promoter sequence;
(ii) a non-translated leader sequence derived from HBV preS2 antigen sequence, HBV e-antigen sequence or HSV type 2 gD antigen sequence; and
(iii) a coding sequence operably linked to (i) and (ii)

wherein the coding sequence is heterologous to the non-translated leader sequence.

The invention also provides a nucleic acid construct comprising:
(i) a promoter sequence;
(ii) a coding sequence operably linked to the promoter sequence (i); and
(iii) an enhancer sequence 3′ of and operably linked to the coding sequence (ii);

wherein the enhancer sequence (iii) is derived from a 3′ UTR of an HBsAg sequence or a 3′ UTR of a simian CMV immediate early gene sequence, and the coding sequence (ii) is heterologous to the 3′ enhancer sequence.

The present invention also provides a nucleic acid construct comprising a promoter sequence and a coding sequence operably linked to the promoter, where the construct further comprises:
(a) a non-translated leader sequence which is derived from HBVpreS2 antigen sequence, HBV e-antigen sequence or HSV type 2 gD antigen sequence, which is in operable linkage with the coding sequence and promoter which is heterologous to the coding sequence; and/or
(b) an enhancer sequence 3′ of and operably linked to the coding sequence, where the enhancer sequence is derived from a 3′ UTR of an HBsAg sequence or a 3′ UTR of a simian CMV immediate early gene sequence, and the coding sequence is heterologous to the 3′ enhancer sequence.

The present invention additionally provides a population of nucleic acid constructs where the population comprises at least two different constructs of the invention.

In another instance, the invention provides a purified isolated chimeric promoter sequence which comprises:
(a) a hCMV immediate early promoter sequence;
(b) exon 1 and at least a part of exon 2 of the hCMV major immediate early gene; and
(c) a heterologous intron provided in place of the intron A region of the hCMV major immediate early gene.

The invention also provides
a method of obtaining expression in mammalian cells of a polypeptide of interest, which method comprises transferring into said cells a nucleic acid construct, a population of nucleic acid constructs or coated particles of the invention;
coated particles, suitable for delivery from a particle-mediated delivery device, which particles comprise carrier particles coated with a nucleic acid construct of the invention, the construct including a coding sequence encoding the polypeptide;
a dosage receptacle for a particle mediated delivery device comprising the coated particles;
a particle mediated delivery device loaded with the coated particles;
a method of nucleic acid immunisation comprising administering to a subject an effective amount of the coated particles of the invention;
a pharmaceutical composition comprising a nucleic acid construct of the invention or a population of nucleic acid constructs of the invention together with a pharmaceutically acceptable carrier or excipient;
a vaccine composition comprising a nucleic acid construct of the invention or a population of nucleic acid constructs of the invention or coated particles of the invention.

Also provided for is the use of a nucleic acid construct of the invention, a population of nucleic acid constructs of the invention or coated particles of the invention in the manufacture of a medicament for nucleic acid immunisation.

These and other objects, aspects, embodiments and advantages of the present invention will readily occur to those of ordinary skill in the art in view of the disclosure herein.

### BRIEF DESCRIPTION OF THE DRAWINGS

FIG. 1 illustrates the levels of expression of hepatitis B virus surface antigen (HBsAg) obtained using various plasmid expression vectors.

FIG. 2 shows the effect of intron inclusion on expression of HBsAg in SCC15 cells and of beta-gal in B16 cells (average of three experiments).

FIG. 3 shows the effect of rat insulin intron A and HBV3′ UTR on expression of beta-gal in SCC15 cells (average of three experiments).

FIG. 4 shows the effect of rat insulin intron A and HBV3′ UTR on expression of HSVgD in SCC15 cells (average of three experiments).

FIG. 5 shows the effect of rat insulin intron A and HBV3′ UTR on expression of SEAP in SCC15 and B16 cells (three repetitions per cell line).

FIG. 6 shows the ability of heterologous signal peptides to direct secretion of SEAP or hFc fragment in B16 cells.

FIG. 7 illustrates levels of antibodies detected in the sera of mice immunised with antigen-encoding nucleic acids contained in a variety of plasmid expression vectors.

FIG. 8 is a diagrammatic representation of the pPJV expression vector.

FIG. 9 is a diagrammatic representation of pPJV7389.

FIG. 10 is a diagrammatic representation of pPJV7400.

FIG. 11 is a diagrammatic representation of pPJV7468.

FIG. 12 is a diagrammatic representation of pPJV7563.

FIG. 13 sets out the base composition for pPJV7563.

FIG. 14 provides a flowchart of the derivitisation of plasmids pPJV7563 and pPJV1671.

FIG. 15 provides maps of the key plasmids in the construction of pPJV1671.

FIG. 16 provides a feature map of pPJV1671 and provides a sequence comparison of the N termini sequences of the natural H3 Panama HA antigen and of the H3 Panama HA antigen encoded by pPJV1671.

FIG. 17 shows the hemagglutination inhibition antibody titres in pigs vaccinated with pPJV1671.

FIG. 18 shows the local skin reactions following particle mediated epidermal delivery of pPJV1671 to humans.

FIG. 19 shows hemagglutination Inhibition antibody titres in pigs immunized with a PMED based trivalent vaccine. HI titers specific for the H3, H1 and B viruses are shown for two PMED devices.

FIG. 20 is a diagrammatic representation of pPML7789.

FIG. 21 shows the annotated sequence of pPML7789.

FIG. 22 is a diagrammatic representation of pPJV2012.

FIG. 23 is a flow chart for the construction of pPJV2012.

FIG. 24 provides the annotated sequence of pPJV2012.

FIG. 25 is a diagrammatic representation of the vector pPJV7788.

FIG. 26 is a diagrammatic representation of the vector pPJV7788 showing restriction sites.

FIG. 27 is a diagrammatic representation of pICP27.

FIG. 28 is the amino acid sequence (SEQ ID NO:65) of ICP27 from MS strain of HSV-2 based on the nucleotide sequence of pICP27. The single amino acid difference between the published sequence of ICP27 from HSV-2 strain MS (asparagine=N) and strain HG52 (lysine) is in bold face type. The sequence identified as the putative CD8 epitope in HSV-2 strain MS is underlined.

FIG. 29 shows the identification of a dominant ICP27 epitope in Balb/c mice. A: Spleen (S) and lymph node (N) cells from HSV-2 infected Balb/c mice (BS and BN, respectively) or C57BL/6 mice (CS and CN) were assayed for IFN-γ ELISPOT activity using various peptide pools (C1-C12, R1-R6) described in Example 22. 5×10⁵ cells were plated into each well and results are tabulated as negative (empty squares), weak (<25 ELISPOTs/well, grey squares) or strong (>25 ELISPOTs/well, black squares). B: Sequences of the two peptides (SEQ ID NOS:66 and 67) recognized by cells from Balb/c mice highlighting a potential HSV-2 Dd epitope (bold) and a region (underlined) corresponding to a previously identified HSV-1 epitope (Banks et al, J. Virol. 67, 613-616, 1993).

FIG. 30 reports characterizing the Balb/c response to ICP27 using an IFN-γ ELISPOT assay. Spleen cells from infected Balb/c mice were assayed for IFN-γ ELISPOT activity using the peptide pool made of all peptides from ICP27 (Pool), or individual peptides HGPSLYRTF (P1, SEQ ID NO:68) and LYRTFAANPRA (P2, SEQ ID NO:69). 5×10⁵ cells were plated into each well and results are expressed as number of ELISPOTs/well. In addition, an aliquot of the spleen cell sample was treated with magnetic beads to deplete the sample of CD8+ cells as described in Example 22. The original sample (+CD8) and depleted sample (−CD8) were assayed for IFN-γ ELISPOT activity using the peptide pool made of all peptides from ICP27 (Pool).

FIG. 31 shows the correlation between HSV-2 challenge protection and ICP27-specific cytokine production. Groups of 16 mice received primary and booster PMED DNA immunizations consisting of the pICP27 DNA vaccine with and without CT DEI vector pPIV2013 (dual A+B subunit) or subunit LT DEI vector pPJV2012 (dual A+B). The pICP27 to DEI vector was a ratio of 9:1. Panel A: post challenge survival data. Half of the animals in each group were challenged two weeks following the second immunization with 50 LD₅₀ of HSV-2. Panels B and C: ICP27-specific IFN-γ and TNF-α production in each group, respectively. The remaining animals were sacrificed at the same time and splenocytes were collected for measurement of ICP27-specific cytokine production using a cytometric bead array kit.

FIG. 32 shows the role of IFN-γ and TNF-α in protection from HSV-2 lethal challenge—morbidity evaluation. Groups of 4 mice received primary and booster immunizations with the pICP27 PMED DNA vaccine with (4 groups) or without (1 group) the pPJV2012 dual A+B LT DEI vector. The pICP27 to DEI vector was a ratio of 9:1. Animals were intranasally challenged two weeks later with 50 LD₅₀ of HSV-2. For T-cell depletion, mice received intraperitoneal injections containing 200 μg of monoclonal antibody specific for IFN-γ and/or TNF-α on days −2, 0, 2, 4, 6, and 8 relative to virus challenge as described in Example 22. Animals were monitored for mortality (% survival) and morbidity, scored on a scale of 0 to 4 according to the following schedule: 4, healthy; 3, ruffled fur, sneezing; 2, sores on eyes or rump, reduced movement; 1, hunched, little movement; 0, dead.

FIG. 33 shows the role of T-cell populations in protection from HSV-2 lethal challenge. Five groups of 4 mice received primary and booster immunizations with the pICP27 PMED DNA vaccine with (+LT, 4 groups) or without (−LT, 1 group) the pPJV2012 dual A+B LT DEI vector. The pICP27 to DEI vector was a ratio of 9:1. Animals were challenged intranasally with 50 LD₅₀ of HSV-2 two weeks later. Three groups of the ICP27+LT DEI-immunized animals were treated with intraperitoneal injections of 90 μg of αCD4 and/or αCD8-specific monoclonal antibodies at, days −2 and 0 relative to virus challenge. In order to keep the total DNA in the pICP27 alone group (indicated as −LT) equal to the pICP27+LT groups, empty vector was added to the pICP27 alone group.

### BRIEF DESCRIPTION OF THE SEQUENCES

SEQ ID NO:1 is hCMV immediate early promoter sequence (GenBank #M60321, X17403)

SEQ ID NO:2 is sequence from exons 1 and 2 of the hCMV major immediate early gene (GenBank #M60321, X17403)

SEQ ID NO:3 is rat insulin intron A sequence (GenBank #J00748)

SEQ ID NO:4 is the sequence of a chimeric promoter according to the present invention

SEQ ID NO:5 is a leader sequence from HBV pre S2 antigen 5′ UTR sequence (GenBank #M54923)

SEQ ID NO:6 is a leader sequence from HSV type 2 gD 5′ UTR sequence (GenBank #Z86099)

SEQ ID NO:7 is a leader sequence from HBV e antigen 5′ UTR sequence (GenBank #M54923)

SEQ ID NO:8 is HBVenh 3′ UTR sequence (GenBank #AF143308)

SEQ ID NO:9 is simian immediate early gene 3′ UTR sequence (GenBank #M16019)

SEQ ID NO:10 is rabbit, globin poly A sequence (GenBank #K03256)

SEQ ID NO:11 is simian sCMV immediate early gene poly A sequence (GenBank #M16019)

SEQ ID NO:12 is HSV2 gB gene poly A sequence (GenBank #Z86099)

SEQ ID NO:13 is HPV16 early gene poly A sequence (GenBank #K02718)

SEQ ID NO:14 is the sequence of the pPJV expression vector

SEQ ID NO:15 is PCR primer JF93

SEQ ID NO:16 is PCR primer F110

SEQ ID NO:17 is PCR primer GW1

SEQ ID NO:18 is PCR primer JF254

SEQ ID NO:19 is PCR primer GW150

SEQ ID NO:20 is PCR primer JF255

SEQ ID NO:21 is PCR primer DS1

SEQ ID NO:22 is PCR primer DA1

SEQ ID NO:23 is PCR primer JF301

SEQ ID NO:24 is PCR primer JF302

SEQ ID NO:25 is PCR primer JF84

SEQ ID NO:26 is PCR primer JF225

SEQ ID NO:27 is PCR primer JF335

SEQ ID NO:28 is PCR primer JF336

SEQ ID NO:29 is PCR primer JF357

SEQ ID NO:30 is PCR primer JF365

SEQ ID NO:31 is PCR primer JF393

SEQ ID NO:32 is PCR primer JF406

SEQ ID NO:33 is PCR primer JF256

SEQ ID NO:34 is PCR primer JF257

SEQ ID NO:35 is PCR primer JF320

SEQ ID NO:36 is PCR primer JF321

SEQ ID NO:37 is PCR primer JF386

SEQ ID NO:38 is PCR primer FcAS

SEQ ID NO:39 is oligonucleotide JF354

SEQ ID NO:40 is PCR primer JF355

SEQ ID NO:41 is PCR primer JF356

SEQ ID NO:42 is oligonucleotide JF348

SEQ ID NO:43 is PCR primer JF349

SEQ ID NO:44 is PCR primer JF350

SEQ ID NO:45 is oligonucleotide JF351

SEQ ID NO:46 is PCR primer JF352

SEQ ID NO:47 is PCR primer JF353

SEQ ID NO:48 is PCR primer JF430

SEQ ID NO:49 is PCR primer JF442

SEQ ID NO:50 is PCR primer JF421

SEQ ID NO:51 is PCR primer JF444

SEQ ID NO:52 is Pseudorabies virus (PRV) promoter sequence.

SEQ ID NO:53 is Rous sarcoma virus (RSV) promoter sequence.

SEQ ID NO:54 provides the nucleotide sequence of the pPJV1671 vector and the amino acid sequence of the encoded H3N2 HA antigen.

SEQ ID NO:55 provides the amino acid sequence alone of the H3N2 HA antigen encode by pPJV1671.

SEQ ID NO:56 provides the natural N terminal amino acid sequence of the H3 Panama HA antigen.

SEQ ID NO:57 provides the N terminal amino acid sequence for the H3 Pananama HA antigen encoded by pPJV1671.

SEQ ID NO:58 provides the consensus sequence of the sequences of SEQ ID Nos. 56 and 57.

SEQ ID NO:59 provides the nucleotide sequence of the pPML7789 vector and the amino acid sequence of the VN1194H5 antigen.

SEQ ID NO:60 provides the amino acid sequence alone of the VN1194H5 antigen.

SEQ ID NO:61 provides the nucleotide sequence of the pPJV2012 vector.

SEQ ID NO:62 provides the nucleotide sequence of the PJV7788 vector.

SEQ ID NOS:63 and 64 are the 5′ and 3′ primers used in Example 22 to amplify DNA of the MS strain of HSV-2.

SEQ ID NO:65 is the amino acid sequence of ICP27 from MS strain of HSV-2 based on the nucleotide sequence of pICP27, Example 22.

SEQ ID NOS:66 and 67 are peptides 45 and 46 recognised by cells from Balb/c mice in Example 22.

SEQ ID NO:68 is a homologous nine amino acid sequence contained in peptides 45 and 46.

SEQ ID NOS:69 and 70 are amino acid regions of HSV-2 ICP27 and HSV-1 ICP27 that differ by one amino acid.

### DETAILED DESCRIPTION OF THE INVENTION

Before describing the present invention in detail, it is to be understood that this invention is not limited to particularly exemplified molecules or process parameters as such may, of course, vary. It is also to be understood that the terminology used herein is for the purpose of describing particular embodiments of the invention only, and is not intended to be limiting. In addition, the practice of the present invention will employ, unless otherwise indicated, conventional methods of virology, microbiology, molecular biology, recombinant DNA techniques and immunology all of which are within the ordinary skill of the art. Such techniques are explained fully in the literature. See, e.g., Sambrook, et al., Molecular Cloning: A Laboratory Manual (2nd Edition, 1989); DNA Cloning: A Practical Approach, vol. I & II (D. Glover, ed.); Oligonucleotide Synthesis (N. Gait, ed., 1984); A Practical Guide to Molecular Cloning (1984); and Fundamental Virology, 2nd Edition, vol. I & II (B. N. Fields and D. M. Knipe, eds.).

All publications, patents and patent applications cited herein, whether supra or infra, are hereby incorporated by reference in their entirety.

It must be noted that, as used in this specification and the appended claims, the singular forms “a,” “an” and “the” include plural referents unless the content clearly dictates otherwise.

In instances where a particular agent is specified as comprising particular units, in a preferred instance the agent may consist essentially of such units.

### A. DEFINITIONS

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In describing the present invention, the following terms will be employed, and are intended to be defined as indicated below.

The term “nucleic acid immunization” is used herein to refer to the introduction of a nucleic acid molecule encoding one or more selected antigens or polypeptides into a host cell for the in vivo expression of the antigen or antigens. The nucleic acid molecule can be introduced directly into the recipient subject, such as by standard intramuscular or intradermal injection; transdermal particle delivery; inhalation; topically, or by oral, intranasal or mucosal modes of administration. In particular, the nucleic acid may be administered via transdermal particle delivery. The molecule alternatively can be introduced ex vivo into cells which have been removed from a subject. In this latter case, cells containing the nucleic acid molecule of interest are re-introduced into the subject such that an immune response can be mounted against the antigen encoded by the nucleic acid molecule. The nucleic acid molecules used in such immunization are generally referred to herein as “nucleic acid vaccines.” Any of the nucleic acids mentioned herein may be present in such vaccines and in particular the nucleic acid constructs mentioned herein may be present.

The term “adjuvant intends an material or composition capable of specifically or non-specifically altering, enhancing, directing, redirecting, potentiating or initiating an antigen specific immune response. Thus, coadminisration of an adjuvant with an antigen may result in a lower dose or fewer doses of antigen being necessary to achieve a desired immune response in the subject to which the antigen is administered, or coadministration may result in a qualitatively and/or quantitavely different immune response in the subject. In particular, the administration of the adjuvant may result in an enhanced immune response such as one of greater magnitude and/or duration. The effectiveness of an adjuvant can be determined by administering the adjuvant with a vaccine composition in parallel with a vaccine composition alone to animals and comparing antibody and/or cellular mediated immunity in the two groups using standard assays such as radioimmunoassay, ELISAs, and CTL assays. The constructs of the invention may express one or more adjuvant polypeptides.

By “core carrier” is meant a carrier on which a guest nucleic acid (e.g., DNA, RNA) is coated in order to impart a defined particle size as well as a sufficiently high density to achieve the momentum required for cell membrane penetration, such that the guest molecule can be delivered using particle-mediated techniques (see, e.g., U.S. Pat. No. 5,100,792). Core carriers typically include materials such as tungsten, gold, platinum, ferrite, polystyrene and latex. See e.g., Particle Bombardment Technology for Gene Transfer, (1994) Yang, N. ed., Oxford University Press, New York, N.Y. pages 10-11.

By “needleless syringe” is meant an instrument which delivers a particulate composition transdermally without the aid of a conventional needle to pierce the skin. Needleless syringes for use with the present invention are discussed herein.

The term “transdermal” delivery intends intradermal (e.g., into the dermis or epidermis), transdermal (e.g., “percutaneous”) and transmucosal administration, i.e., delivery by passage of an agent into or through skin or mucosal tissue. See, e.g., Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987). Thus, the term encompasses delivery from a needleless syringe as described in U.S. Pat. No. 5,630,796, as well as particle-mediated delivery as described in U.S. Pat. No. 5,865,796.

A “polypeptide” is used in it broadest sense to refer to a compound of two or more subunit amino acids, amino acid analogs, or other peptidomimetics. The subunits may be linked by peptide bonds or by other bonds, for example ester, ether, etc. As used herein, the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is typically called a polypeptide or a protein.

An “antigen” refers to any agent, generally a macromolecule, which can elicit an immunological response in an individual. The term may be used to refer to an individual macromolecule or to a homogeneous or heterogeneous population of antigenic macromolecules. As used herein, “antigen” is generally used to refer to a protein molecule or portion thereof which contains one or more epitopes. An “antigen” may comprise, for instance, a naturally occurring polypeptide, a fragment of such a polypeptide which is immunogenic or a variant form of either which retains immunogenicity. In a preferred instance, where a fragment or variant is referred to, the immune response generated may preferably be capable of recognizing the original polypeptide from which the fragment or variant is derived.

For the purposes of the present invention, antigens can be obtained or derived from any appropriate source. For the purposes of the present invention, an “antigen” includes a protein having modifications, such as deletions, additions and substitutions (generally conservative in nature) to the native sequence, so long as the protein maintains sufficient immunogenicity. These modifications may be deliberate, for example through site-directed mutagenesis, or may be accidental, such as through mutations of hosts which produce the antigens. In a particularly preferred embodiment of the invention the antigen employed or encoded may be an influenza antigen, an immunogenic fragment of an influenza antigen or an immunogenic variant of either.

An “immune response” against an antigen of interest is the development in an individual of a humoral and/or a cellular immune response to that antigen. For purposes of the present invention, a “humoral immune response” refers to an immune response mediated by antibody molecules, while a “cellular immune response” is one mediated by T-lymphocytes and/or other white blood cells.

The terms “nucleic acid molecule” and “polynucleotide” are used interchangeably herein and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term nucleic acid sequence is the alphabetical representation of a polynucleotide molecule. This alphabetical representation can be input into databases in a computer having a central processing unit and used for bioinformatics applications such as functional genomics and homology searching.

A “vector” is capable of transferring nucleic acid sequences to target cells (e.g., viral vectors, non-viral vectors, particulate carriers, and liposomes). The target cells may be procaryotic or eucaryotic. Typically, “vector construct,” “expression vector,” and “gene transfer vector,” mean any nucleic acid construct capable of directing the expression of a gene of interest and which can transfer gene sequences to target cells. Thus, the term includes cloning and expression vehicles, as well as viral vectors. A “plasmid” is a vector in the form of an extrachromosomal genetic element.

A nucleic acid sequence which “encodes” a selected antigen is a nucleic acid molecule which is transcribed (in the case of DNA) and translated (in the case of mRNA) into a polypeptide in vivo or in vitro when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, procaryotic or eucaryotic mRNA, genomic sequences from viral or procaryotic DNA or RNA, and even synthetic DNA sequences. A transcription termination sequence may be located 3′ to the coding sequence.

In some cases a transcribed sequence may give rise to multiple polypeptides, for instance a transcript may contain multiple open reading frames (ORFs) and also one or more Internal Ribosome Entry Sites (IRES) to allow translation of ORFs after the first ORF. A transcript may be translated to give a polypeptide which is subsequently cleaved to give a plurality of polypeptides. In some cases a nucleic acid construct may give rise to multiple transcripts and hence a plurality of polypeptides.

A “promoter” is a nucleotide sequence which initiates and regulates transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

“Operably linked” refers to an arrangement of elements wherein the components so described are configured so as to perform their usual function. Thus, a given promoter operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence.

“Recombinant” is used herein to describe a nucleic acid molecule (polynucleotide) of genomic, cDNA, semisynthetic, or synthetic origin which, by virtue of its origin or manipulation is not associated with all or a portion of the polynucleotide with which it is associated in nature and/or is linked to a polynucleotide other than that to which it is linked in nature. Two nucleic acid sequences which are contained within a single recombinant nucleic acid molecule are “heterologous” relative to each other when they are not normally associated with each other in nature.

Homologues of polynucleotides are referred to herein. Typically a polynucleotide which is homologous to another polynucleotide is at least 70% homologous to the polynucleotide, preferably at least 75, 80 or 90% and more preferably at least 95%, 97% or 99% homologous thereto. Methods of measuring homology are well known in the art and it will be understood by those of skill in the art that in the present context, homology is calculated on the basis of nucleic acid identity. Such homology may, for instance, exist over a region of at least 15, preferably at least 30, for instance at least 40, 60 or 100 or more contiguous nucleotides. The region of homology may be over at least 150, preferably at least 200 and even more preferably over at least 300 nucleotides. The region of homology may relate to any of the elements referred to herein in relation to the nucleic acid constructs of the invention. In some cases, the region of homology may be over the entire region in question, such as, for instance, over the entire region of any of the elements specified herein.

Equivalent levels of amino acid homology to those referred to in relation to nucleotide homology above may be present. Thus, any of the above mentioned levels of homology may apply at the amino acid level. Homology at the amino acid level may, for instance, be over at least 15, preferably at least 25, more preferably over at least 50, still more preferably at least 75 and even more preferably over at least 100 amino acids. The region of homology may be over the entire length of the element in question.

Methods of measuring polynucleotide homology or identity are known in the art. For example the UWGCG Package provides the BESTFIT program which can be used to calculate homology (e.g. used on its default settings) (Devereux et al (1984) Nucleic Acids Research 12, p 387-395).

The PILEUP and BLAST algorithms can also be used to calculate homology or line up sequences (typically on their default settings), for example as described in Altschul S. F. (1993) J Mol Evol 36:290-300; Altschul, S, F et al (1990) J Mol Biol 215:403-10.

Software for performing BLAST analysis is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89:10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl, Acad. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologues typically hybridize with the relevant polynucleotide at a level significantly above background. The signal level generated by the interaction between the homologue and the polynucleotide is typically at least 10 fold, preferably at least 100 fold, as intense as “background hybridisation”. The intensity of interaction may be measured, for example, by radiolabelling the probe, e.g. with ³²P. Selective hybridisation is typically achieved using conditions of medium to high stringency, (for example, 0.03M sodium chloride and 0.003M sodium citrate at from about 50° C. to about 60° C.

Stringent hybridization conditions can include 50% formamide, 5× Denhardt's Solution, 5×SSC, 0.1% SDS and 100 μg/ml denatured salmon sperm DNA and the washing conditions can include 2×SSC, 0.1% SDS at 37° C. followed by 1×SSC, 0.1% SDS at 68° C. Defining appropriate hybridization conditions is within the skill of the art. See, e.g., Sambrook et al., supra.

The homologue may differ from a sequence in the relevant polynucleotide by less than 3, 5, 10, 15, 20 or more mutations (each of which may be a substitution, duplication, deletion or insertion). These mutations may be measured over a region of at least 30, for instance at least 40, 60 or 100 or more contiguous nucleotides of the homologue. In some instances the mutations may be measured over the entire region of the homologue. Where a polynucleotide encodes a polypeptide, substitutions preferably create “conservative” changes in the amino acid encoded. These are defined according to Table 1 below. Amino acids in the same block in the second column and preferably in the same line in the third column may be substituted for each other in conservative changes.

TABLE 1

| | | |
|---|---|---|
| ALIPHATIC | Non-Polar | G A P |
| | | I L V |
| | Polar-uncharged | C S T M |
| | | N Q |
| | Polar-charged | D E |
| | | K R |
| AROMATIC | | H F W Y |

The term “fragment” indicates a smaller part of a larger entity. Fragments of specific elements referred to herein may be employed in the invention. In particular, such fragments will retain some or all functionality of the original element and in particular any of the functions mentioned herein. In preferred instances a fragment of an antigen may retain immunogenicity and a fragment of an adjuvant the ability to act as an adjuvant. In some instances, a fragment may be at least 50%, preferably at least 60%, more preferably at least 70%, still more preferably at least 80%, even more preferably at least 90% and still more preferably at least 95% of the length of the original. A fragment may be equal to or less than such percentages of the length of the original.

The terms “individual” and “subject” are used interchangeably herein to refer to any member of the subphylum cordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs as well as pigs; birds, including domestic, wild and game birds such as chickens, turkeys and other gallinaceous birds, ducks, geese, and the like. The terms do not denote a particular age. Thus, both adult and newborn individuals are intended to be covered. The methods described herein are intended for use in any of the above vertebrate species, since the immune systems of all of these vertebrates operate similarly.

In some instances, the invention may be used to immunize any suitable subject and in particular any suitable subject of a given species. In a preferred instance, any suitable human subject may be immunized. Thus, as many subjects as possible may, for instance, be immunized without emphasis on any particular group of subjects. For instance, a population of subjects as a whole, or as many as possible, may be immunized. In particular, where the invention is being used to treat influenza, and especially to immunize against a pandemic strain of influenza, the constructs of the invention may be used to immunize any subject and preferably as many subjects as possible.

In other cases, the subject or individual may be one at risk of infection or for whom infection would be particularly detrimental. The infection may, in a preferred instance, be a respiratory infection. In particular, where the invention is being used to prevent or treat a respiratory infection and in particular against influenza, the subject may be human. In some cases, the nucleic acid constructs of the invention may be administered in preference, or first, to particular at risk groups. This may, for instance, be the case for administration of constructs to immunize against non-pandemic strains of influenza. In some cases the subjects may, for instance, fall into one or more of the following categories:
a subject with a respiratory disorder and/or heart problems and in particular who has asthma, emphysema, bronchitis and/or chronic obstructive pulmonary disease (COPD);
a subject with a chronic medical condition such as diabetes, immune suppression, immune deficiency, sickle cell disease and/or a kidney disease;
a subject aged at least 50 years, preferably at least 60 years, more preferably at least 65 years, even more preferably at least 75 years of age and still more preferably at least 80 years of age;
a child aged 2 years or less, in particular from 6 to 23 months, for instance 18 months or less;
a subject on a chronic aspirin therapy and in particular one aged from 6 months to 18 years;
a pregnant woman and in particular one who will be in their second or third trimester of pregnancy during influenza season;
a resident of a nursing home or long term care facility; and/or
a care worker for any of the above or someone who is likely to come into regular contact with them.

### B. GENERAL OVERVIEW

The invention is concerned with nucleic acid constructs which allow efficient expression of heterologous coding sequences, and in particular antigen-encoding genes, in host cells. The constructs may, in some instances, express one or more adjuvant polypeptides. More specifically, the invention provides nucleic acid constructs comprising, or in some embodiments, consisting essentially of a chimeric promoter sequence and a cloning site, such that when a coding sequence is inserted in the cloning site, the coding sequence is in operable linkage with the chimeric promoter. The invention also provides constructs with coding sequences inserted into the cloning site or sites. The coding sequences may encode any of the polypeptides referred to herein and in particular an antigen and especially any of the antigens and adjuvant polypeptides mentioned herein.

In an especially preferred embodiment the constructs comprise a coding sequence and in particular a coding sequence encoding an antigen, an immunogenic fragment thereof or an immunogenic variant of either. In an especially preferred embodiment the coding sequences encode an influenza antigen, an immunogenic fragment thereof or an immunogenic variant of either. In a further preferred embodiment the coding sequence may encode an adjuvant polypeptide and in particular an ADP ribosylating bacterial toxin subunit, a fragment thereof with adjuvant activity or a variant of either with adjuvant activity.

The chimeric promoter comprises, or in some embodiments consists essentially of:
(a) a hCMV immediate early promoter sequence;
(b) exon 1 and at least a part of exon 2 of the hCMV major immediate early gene; and
(c) a heterologous intron provided in place of the intron A region of the hCMV major immediate early gene.

The hCMV immediate early promoter sequence (a) may comprise:
(i) a native hCMV immediate early promoter sequence;
(ii) a functional homologous variant thereof; or
(iii) a functional fragment of (i) or (ii).

In general sequence (a) comprises about 100 to 600, preferably 200 to 600, for instance 400 to 600 nucleotides. Typically sequence (a) comprises the sequences present in (i) which bind transcription factors or the RNA polymerase, or instead of any of these sequences, homologues of these sequences able to bind the same transcription factors and RNA polymerase. Typically such sequences or their homologues are present in promoter sequence (a) in the same order and/or substantially the same relative spacing as in (i).

Generally, (i) comprises at least from nucleotides −100 to −1, typically −150 to −1, for example −500 to −1 or −600 to −1 of the hCMV major immediate early gene. Sequence (i) typically comprises the hCMV core promoter sequence and may also include one or more enhancer elements present in hCMV immediate early promoter. For example, (i) may comprise from nucleotides −118 to −1, or −524 to −1 as in U.S. Pat. No. 6,218,140, or from nucleotides −62 to 1 or −465 to −1 as in U.S. Pat. No. 5,385,839.

Generally (i) includes a TATA box or CAAT box commonly found in promoter sequences. Preferably the sequence includes one or more of the repeat sequences in the hCMV immediate early promoter.

In a preferred embodiment, (i) comprises SEQ ID NO: 1. In a further preferred embodiment, (i) comprises nucleotides 903 to 1587 of SEQ ID NO: 54. In another embodiment (i) may comprise nucleotides 903 to 1587 of SEQ ID NO: 14. In a further preferred embodiment (i) may comprise nucleotides 1002 to 1686 or 2624 to 3308 of SEQ ID No. 57. In a further preferred embodiment (i) may comprise nucleotides 1815 to 1935 and/or 1948 to 2632 of SEQ ID No. 61 and in particular nucleotides 1948 to 2632 of SEQ ID No. 61. In another preferred embodiment nucleotides 1815 to 1935 may be used.

In a particularly preferred embodiment of the invention the hCMV immediate early promoter sequence (a) comprises:
(i) the nucleotide sequence of SEQ ID No. 1, nucleotides 903 to 1587 of SEQ ID No. 54, nucleotides 1815 to 1935 of SEQ ID No: 61, nucleotides 1948 to 2632 of SEQ ID No: 61, nucleotides 1002 to 1686 of SEQ ID No: 62 and/or nucleotides 2624 to 3308 of SEQ ID No: 62;
(ii) a functional variant of (i) which has at least 80% nucleotide sequence homology to one or more of the sequences of (i); and/or
(iii) a functional fragment of (i) or (ii).

In some instances, a fragment may comprise at least 300 nucleotides, preferably at least 400 nucleotides, more preferably at least 500 nucleotides and even more preferably at least 600 nucleotides of such sequences. The fragment may comprise up to 800, up to 600 or up to 400 nucleotides

A hCMV immediate early promoter sequence can be obtained using known methods. A native hCMV immediate early promoter can be isolated directly from a sample of the virus, using standard techniques. U.S. Pat. No. 5,385,839, for example, describes the cloning of a hCMV promoter region. The sequence of a hCMV immediate early promoter is available at Genbank #M60321 (hCMV Towne strain) and X17403 (hCMV Ad169 strain). A native sequence could therefore be isolated by PCR using PCR primers based on the known sequence. See e.g. Sambrook et al, supra, for a description of techniques used to obtain and isolate DNA. A suitable hCMV promoter sequence could also be isolated from an existing plasmid vector. Promoter sequences can also be produced synthetically.

A functional variant (ii) or fragment (iii) is generally one which retains and/or complements the activity of the native promoter (i). Typically this activity is the ability to cause (including initiating and regulating) transcription of an operably linked polynucleotide, in particular the hCMV major immediate early gene. In one embodiment, the variant or fragment would be able to complement the activity of the native promoter in a hCMV virus, for example allowing the virus to retain the ability to infect and/or replicate in cells.

A homologous variant (ii) or fragment (iii) can be assayed for the ability to retain and/or complement the activity of (i). For example, a variant or fragment may be assayed for ability to restore functionality (such as infection and/or replication ability) to mutant hCMV in which the native hCMV immediate early promoter is defective.

A homologous variant (ii) or fragment (iii) may be tested for utility using the Comparative Expression Assay below. The test promoter sequence is swapped into the base vector in place of the native hCMV immediate early promoter. Typically, a functional variant or fragment allows at least 50%, for instance, 60, 70, 80, 90% or more of the expression provided using the base vector. A functional variant or fragment may provide any of the levels of expression referred to herein. In some instances, the variant or fragment may provide a higher level of expression such as at least 50%, 100%, 150%, 200%, 300% or more increase in activity. Generally expression is provided in at least one, but preferably two, reference cell types. Typically, the reference cells are mammalian HEK 293T, CHO, HeLa, BHK, 3T3 or COS cells. The reference cells may be SSC-15 or B16 host cells in some instances.

Additionally or alternatively, promoter sequence may be tested in the Comparative Immunogenicity Assay below. Test promoter sequence is swapped into the base vector in place of the native hCMV immediate early promoter. A functional promoter sequence typically provides antibody titres that are at least as high as or higher than those achieved by the base vector with at least one, preferably two antigens. Preferably antibody titres are at least 5%, 10%, 20%, 30% or 40% higher than with the base vector. In some cases, the level of antigen titres may be any of those referred to herein. Suitable antigens are HBsAg, HSV 2gD and flu-M2 antigens. Particularly preferred antigens are influenza antigens. Influenza antigens include the HA, NA, M2, NP, M1 PB1, PB2, PA, NS1 and NS2 antigens and in particular the HA, NA and M2 antigens. In an especially preferred embodiment the antigen is HA or a fragment thereof or a variant of either. According to the assay, a functional homologous variant (ii) or functional fragment (iii) of native promoter sequence (i) is typically one which allows the highest antibody titres achieved by the native sequence. In some instances, the antibody titre may be slightly lower including any of the levels mentioned herein.

In instances where a construct of the invention encodes an adjuvant polypeptide, the comparative immunogenicity test may be employed with a standard antigen and comparing a test vector encoding a polypeptide of unknown adjuvant activity with a standard adjuvant vector. For instance, a test adjuvant vector may encode a fragment or variant of a known adjuvant polypeptide and be compared against a standard vector expressing the known adjuvant polypeptide in their ability to promote an immune response when administered with an antigen. A fragment or variant may have any of the levels of activity mentioned herein and in particular will provide at least 50%, preferably at least 75%, more preferably at least 85% and even more preferably at least 100% of the adjuvant activity of the standard adjuvant. Preferably the test vector and standard vector will be identical, or at least substantially identical, apart from the sequences encoding the adjuvant/polypeptide under test.

As mentioned above, the construct may comprise exon sequence (b) which comprises sequence derived from exon 1 and exon 2 of the hCMV major immediate early gene. Exons are coding sequences, which in nature are generally separated by introns. In the native hCMV major immediate early gene, exons 1 and 2 are usually separated by the native intron A. In the present chimeric construct exon 2 sequence is generally positioned 3′ of exon 1 sequence, without intervening intron sequence, so that the exon 1 and exon 2 sequences are contiguous.

Exon sequence (b) may comprise:
(i) native exon sequence, typically exon 1 and (whole or partial) exon 2;
(ii) a homologous variant of (i) which is functional; or
(iii) a functional fragment of (i) or (ii).

Sequence (i) may comprise from about 50 to 100% of the native hCMV major immediate early gene exon 1 sequence, for example, 60 to 90% or 70 to 80%. Typically at least 50% of the natural exon 1 sequence is present, such as 60%, 70%, 80%, 90% or more. Exon sequence (b) also comprises at least a part of exon 2 sequence. In sequence (i), typically 2 or more bases of native exon 2, for example 2 to 9, 2 to 7 or 3 to 5 bases are present. Up to and including 100% of natural exon 2 sequence, for example 5 to 95%, 20 to 80% or 40 to 60% of natural exon 2 sequence may be present. Typically the homologous variant has any of the above lengths mentioned for the native sequence.

Preferably (i) comprises SEQ ID No. 2. In a further preferred embodiment (i) comprises nucleotides 1588 to 1718 of SEQ ID NO: 54. In a further preferred embodiment (i) comprises nucleotides 1588 to 1718 of SEQ ID No. 14. In another preferred instance (i) comprises nucleotides 1684 to 1814 and/or 2633 to 2763 of SEQ ID No. 51. In a further preferred embodiment, (i) may comprise nucleotides 1687 to 1817 and/or 3309 to 3439 of SEQ ID No: SEQ ID No. 62.

Thus, in a particularly preferred embodiment exon sequence (b) of the chimeric promoter comprises:
(i) the nucleotide sequence of SEQ ID No. 2, the nucleotide sequence of nucleotides 1588 to 1718 of SEQ ID No. 54, nucleotides 1684 to 1814 of SEQ ID No: 61, nucleotides 2633 to 2763 of SEQ ID No: 61, nucleotides 1687 to 1817 of SEQ ID No: 62 and/or nucleotides 3309 to 3439 of SEQ ID No: 62;
(ii) a functional variant of (i) which has at least 80% nucleotide sequence homology to one or more of the sequences of (i); and/or
(iii) a functional fragment of (i) or (ii).

Suitable exon sequence (b) can be obtained using known methods. See e.g. Sambrook et al., supra, for a description of techniques used to obtain and isolate DNA. Native hCMV major immediate early gene sequence can be isolated directly from a sample of the virus, using standard techniques (see for example, MacLean, A (1998) “Preparation of HSV-DNA and Production of Infectious Virus” in Herpes Simplex Virus Protocols S. Brown, A Maclean, editors, Humana Press, Totowa, N.J., pp. 19-26). The sequence of a hCMV major immediate early gene, including the location of exon 1 and exon 2, is available at Genbank #M60321, X17403. Native exon 1 and 2 sequences could therefore be isolated by cutting the native major gene sequence at appropriate restriction sites or by PCR using PCR primers based on the known sequence. Suitable exon sequences could alternatively be isolated from an existing plasmid vector, such as pWRG7128. Exon sequences can also be produced synthetically, rather than cloned. Variant sequences can readily be constructed by routine methodologies such as site-directed mutagenesis.

Generally the exon sequence will, when present in the construct of the invention, enhance expression, typically causing comparable enhancement to the native exon 1 and exon 2 sequence (i) mentioned above.

Exon sequence may be assayed for functionality using the Comparative Expression Assay below. Test exon sequence is swapped into the base vector in place of the exon sequence already present. Generally exon sequence is functional if the sequence does not abrogate expression, but preferably increases expression in at least one, preferably two reference cell types when compared to the base vector. Typically the reference cells are mammalian HEK293T, CHO, HeLa, BHK, 3T3 or COS cells. SSC15 or B16 cells may be used. Preferably expression increases by at least 5%, 10%, 20%, 30% or 40%. Expression may increase by any of the levels mentioned herein. According to this assay, a functional homologous variant (ii) or functional fragment (iii) of natural exon sequence (i) is one which typically allows at least 50% of the expression improvement provided by the natural sequence. In some instances, where the level of expression is lower than that given by the base level, the decrease may be any of the levels mentioned herein.

Additionally or alternatively, exon sequence may be tested in the Comparative Immunogenicity Assay below. Test exon sequence is swapped into the base vector in place of the exon sequence already present. Functional exon sequence provides antibody titres that are typically at least as high as or higher than those achieved by the base vector with at least one, preferably two antigens. Preferably antibody titres are at least 5%, 10%, 20%, 30% or 40% higher than with the base vector. The increase may be any of the levels mentioned herein. Preferred antigens are HBsAg, HSV 2gD and flu-M2 antigens. Especially preferred antigens are influenza antigens, including any of those mentioned herein and in particular HA, NA and M2 antigens. The use of HA antigen, an immunogenic fragment thereof or an immunogenic variant of either is especially preferred. According to this assay, a functional homologous variant (ii) or functional fragment (iii) of natural exon sequence (i) is one which typically allows the highest antibody titres achieved by the natural sequence. In instances where the level of antibody titre is slightly lower, the decrease may be any of the levels mentioned herein. Adjuvant vectors may be assessed in an equivalent manner as discussed above.

The chimeric promoter construct comprises heterologous intron (c) in place of the native intron A region of the hCMV major immediate early gene. An intron is a non-coding sequence which is spliced from the hnRNA transcribed from a gene. A heterologous intron is one which is not present in the coding sequence in nature.

The heterologous intron (c) replaces wholly or partially, native intron A of the hCMV major immediate early gene. Typically the native intron A is absent.

In general the heterologous intron (c) is 3′ of exon sequence (b).

Typically the heterologous intron (c) comprises:
(i) a natural intron;
(ii) a functional homologous variant of (i); or
(iii) a functional fragment of (i) or (ii).

Heterologous intron (c) is in general a viral or eukaryotic intron. Preferably the intron is a mammalian intron, in particular a non-human intron, for example a rat intron may be employed. In some instances, a chicken intron may be employed. Preferably the intron is an intron A, for example, rat insulin intron A, chicken keratin intron A or chicken cardiac actin intron A. In an especially preferred embodiment the intron is rat insulin intron A.

In a preferred embodiment the heterologous intron (c) comprises a sequence selected from the rat insulin gene intron A sequence, chicken keratin gene intron A sequence, chicken cardiac actin gene intron A sequence, a functional fragment of any thereof or a functional variant of any of the preceding

Typically intron (c) has a length of from about 50 nucleotides to about 1000 nucleotides, for instance from about 100 to about 500 nucleotides. The intron (c) may for example, comprise 50 to 500 nucleotides, such as up to 100, 200, 300 or 400 nucleotides. Preferably the intron comprises sequence found at about nucleotides 50 to 133 of native rat insulin intron A, or a homolog of this sequence.

Preferably heterologous intron (c) is capable of being spliced from an RNA transcript in a eukaryotic host cell. In general the intron comprises one or more of a donor sequence (such as GT), an acceptor sequence (such as AG), a 3′ pyrimidine rich region and a branch point sequence. The pyrimidine rich region, if present, may include, for example at least 5, 8, 10 or more pyrimidines. Preferably the intron comprises at least a donor sequence, acceptor sequence and a branch point sequence. Typically in the chimeric construct, intron (c) comprises non-intron flanking sequences which are derived from exon sequences found on the intron/exon boundaries of the natural intron (i). The flanking exon sequence may be native exon sequence or a homologue of this sequence which retains substantially the same activity as the native sequence, for example retains splicing function. Typically from 5 to 10, preferably from 7 to 10 bases of exon sequence are included at each end of the intron.

Intron (c) may be an artificial intron, provided that the intron is functional. For example, a recombinant or chimeric intron may be used. Such an intron may comprise sequence from more than one natural intron.

Typically intron (c) comprises sequences present in hCMV intron A which bind transcription factors or regulatory proteins or instead of any of these sequences, homologues of these sequences able to bind the same factors or proteins. Typically such sequences or their homologues are present in the intron (c) in the same order and/or substantially the same relative spacing as in hCMV intron A.

Intron (c) may comprise an homologous variant (ii) in which the sequence of the natural intron (i) has been modified to remove an internal restriction site. For example, an homologous variant of rat insulin intron A may be used in which an internal Nhe1 site has been destroyed.

Preferably, intron (c) comprises:
(i) SEQ ID No. 3;
(ii) a functional homologous variant of (i); or
(iii) a functional fragment of (i) or (ii).

In a further preferred instance, (i) may comprise nucleotides 1725 to 1857 of SEQ ID NO: 54 or the same nucleotides of SEQ ID No. 14. In a further preferred embodiment (i) may comprise nucleotides 1545 to 1677 and/or 2770 to 2902 of SEQ ID No: 61. In another preferred instance, (i) may comprise nucleotides 1824 to 1956 and/or 3446 to 3578 of SEQ ID No:62.

In one preferred instance, the rat insulin gene intron A sequence comprises:
(i) the nucleotide sequence of SEQ ID No. 3, the nucleotide sequence of nucleotides 1725 to 1857 of SEQ ID No. 54, nucleotides 1545 to 1677 of SEQ ID No: 61, nucleotides 2770 to 2902 of SEQ ID No: 61, nucleotides 1824 to 1956 of SEQ ID No:62 and/or nucleotides 3446 to 3578 of SEQ ID No:62;
(ii) a functional variant of (i) which has at least 80% nucleotide sequence homology to one or more of the sequences of (i); and/or
(iii) a functional fragment of (i) or (ii).

Intron sequence (c) may be obtained using standard cloning techniques. For example, rat insulin intron A sequence is available at GenBank J00748, chicken keratin intron A sequence at GenBank J00847 and chicken cardiac intron A sequence at GenBank X02212. Intron sequence can be isolated from natural sources using primers based on known sequence. Sequence may be prepared synthetically. Variant sequences may be obtained by mutagenesis.

Typically a functional intron sequence, for example a functional variant (ii) or a functional fragment (iii) is one which has substantially the same activity as, and/or complements the activity of a natural intron (i). In one embodiment the activity is splicing activity.

Intron (c) sequences may be tested for splicing activity using a routine splicing assay. In general a functional homologue (ii) or functional fragment (iii) will show at least 50%, for example 60%, 70%, 80%, 90% and up to 100% or more of the splicing efficiency of the natural intron (i) in the assay.

In general the heterologous intron sequence will, when present in the construct of the invention, enhance expression. Typically, a variant (ii) or fragment (iii) intron will cause comparable enhancement to a natural intron (i). In some cases, a decrease in expression might be seen including any of the levels mentioned herein.

Functionality of potential intron sequence (c) can be tested using the Comparative Expression Assay below. The heterologous intron is swapped into the base vector. Generally heterologous intron sequence is functional if the addition of the sequence increases expression in at least one, but preferably two reference cell types by 25% or more, compared to the base vector. Typically the reference cells are mammalian HEK293T, CHO, HeLa, BHK, 3T3 or COS cells. SSC15 or B16 cells may be used. The increase in expression may be at least 35%, 45%, 55% or more. The increase may be any of the levels mentioned herein. According to this assay, a functional variant (i) or functional fragment (ii) of a natural intron sequence (i) is typically one which allows greater than 50% of the expression improvement achieved by the natural sequence. In cases where a decrease in expression is seen the decrease may, for instance, be any of the levels mentioned herein.

A heterologous intron (c) sequence may, additionally or alternatively be tested for functionality using the Comparative Immunogenicity Assay below. Intron (c) sequence is added to the base vector. A functional intron (c) sequence provides antibody titres that are higher than those achieved by the base vector with at least one, preferably two antigens. Preferably, the antibody titres are at least 5 or 10%, for instance 20%, 30% or 40% higher than with the base vector. Preferred antigens are HBsAg, HSV2gD and Flu-M2 antigens. Especially preferred antigens are influenza antigens, including any of those mentioned herein and in particular HA, NA and M2 antigens. In particular, HA antigen, an immunogenic fragment thereof or a immunogenic variant of either may be employed. According to this assay, a functional variant (ii) or functional fragment (iii) of a natural intron sequence (i) is typically one which allows the highest antibody titres achieved by the natural sequence. In some cases a decrease might be seen, the levels may, for instance, be any of those mentioned herein. Adjuvant vectors may be assessed in an equivalent manner as discussed elsewhere herein.

Suitable heterologous intron sequence can be obtained using standard cloning techniques. For example, rat insulin intron A sequence is available at GenBank J00748, chicken keratin intron A at GenBank J00847, and chicken cardiac actin intron A at X02212. Intron sequence can be isolated from native sources using primers based on the known sequence data. Suitable sequence may also be prepared synthetically.

The component sequences (a), (b) and (c) may be provided suitably linked together to form a chimeric promoter using standard cloning or molecular biology techniques. Preferably intron sequence (c) is provided 3′ of exon sequence (b). The chimeric promoter construct is linked to a cloning site, in such a way that the promoter will effect the expression of a coding sequence inserted in the site, when the proper enzymes are present. Suitable cloning sites, including multi-cloning sites are known in the art, e.g. the pUC19, pBC SK, pBluescript II KS, cDNA3.1, pSP72, pGEM 7Z multicloning site. Any suitable restriction site may, for instance, be used as a cloning site to insert the coding sequences.

In one preferred embodiment, the chimeric promoter employed comprises:
(i) the nucleotide sequence of SEQ ID No. 4 or the nucleotide sequence of nucleotides 903 to 1857 of SEQ ID No. 54;
(ii) a functional variant of (i) which has at least 80% nucleotide sequence homology to (i); and/or
(iii) a functional fragment of (i) or (ii).

In a further preferred instance, the chimeric promoter employed may be one that comprises the sequence of SEQ ID No: 14.

Typically, a nucleic acid for insertion (or inserted) in the cloning site encodes a therapeutically relevant polypeptide. It is preferred that the coding sequence is suitable for use in nucleic acid immunisation or gene therapy. The nucleic acid insert may thus comprise a sequence capable of providing immunity, for example an immunogenic sequence that elicits a humoral and/or cellular immune response when delivered to a subject. Alternatively, the nucleic acid may comprise one or more genes encoding a therapeutic polypeptide e.g. a protein defective or missing from a target cell genome or a non-native protein having a desired biological or therapeutic effect (e.g., an antiviral function). The construct may encode an adjuvant polypeptide. For the treatment of genetic disorders, functional genes corresponding to genes known to be deficient in the particular disorder can be administered to a subject. Preferably the nucleic acid is DNA. The nucleic acid may in some instances be RNA or PNA.

In some instances a non-coding RNA may be expressed by a construct of the invention. Thus the vector may have inserted into the cloning site a region which can give rise to such an RNA, for instance an anti-sense RNA or SiRNA. The anti-sense RNA or SiRNA may, for instance, inhibit the expression of any of the genes mentioned herein or a gene of any of the pathogens mentioned herein. However, in the most preferred embodiment, a polypeptide is encoded.

Suitable nucleic acids for insertion include those used for the treatment of inflammatory diseases, autoimmune, chronic and infectious diseases, including such disorders as AIDS, cancer, neurological diseases, cardivascular disease, hypercholestemia; various blood disorders including various anemias, thalassemia and hemophilia; genetic defects such as cystic fibrosis, Gaucher's Disease, adenosine deaminase (ADA) deficiency, emphysema, etc.

The constructs in the invention may be used to treat or prevent a condition. The constructs may be used to ameliorate a condition and/or eliminate or reduce a particular, or all, symptoms of a disorder. In a particularly preferred instance the constructs may be used to vaccinate a subject against a pathogen.

For example, in methods for the treatment of solid tumors, genes encoding toxic peptides (i.e., chemotherapeutic agents such as ricin, diptheria toxin and cobra venom factor), tumor suppressor genes such as p53, genes coding for mRNA sequences which are antisense to transforming oncogenes, antineoplastic peptides such as tumor necrosis factor (TNF) and other cytokines, or transdominant negative mutants of transforming oncogenes, can be inserted for expression at or near the tumor site.

In one preferred embodiment a construct of the invention may encode a polypeptide for treating or preventing a cancer. In a particularly preferred embodiment a construct of the invention may encode a tumour antigen. Examples of tumour associated antigens include, but are not limited to, cancer-testes antigens such as members of the MAGE family (MAGE 1, 2, 3 etc), NY-ESO-1 and SSX-2, differentation antigens such as tyrosinase, gp100, PSA, Her-2 and CEA, mutated self antigens and viral tumour antigens such as E6 and/or E7 from oncogenic HPV types. Further examples of particular tumour antigens include MART-1, Melan-A, p97, beta-HCG, GalNAc, MAGE-1, MAGE-2, MAGE-4, MAGE-12, MUC1, MUC2, MUC3, MUC4, MUC18, CEA, DDC, P1A, EpCam, melanoma antigen gp75, Hker 8, high molecular weight melanoma antigen, K19, Tyr1, Tyr2, members of the pMl 17 gene family, c-Met, PSM (prostate mucin antigen), PSMA (prostate specific membrane antigen), prostate secretary protein, alpha-fetoprotein, CA125, CA19.9, TAG-72, BRCA-1 and BRCA-2 antigen.

Examples of particular cancers that the antigen may be derived include those from cancers of the lung, prostate, breast, colon, ovary, testes, bowel, melanoma, a lymphoma and a leukaemia. The constructs of the invention may also be used to treat or prevent such cancers.

Similarly, nucleic acids coding for polypeptides known to display antiviral and/or antibacterial activity, or stimulate the host's immune system, can also be included. The nucleic acid may encode one of the various cytokines (or functional fragments thereof), such as the interleukins, interferons and colony stimulating factors.

In a preferred instance, the coding sequences may encode an antigen, immunogenic fragment thereof or immunogenic variant of either. The antigen may in particular be a viral, bacterial, parasitic or fungal pathogen antigen or a tumour antigen. In a preferred instance, the antigen is a viral antigen, an immunogenic fragment thereof or an immunogenic variant of either.

The nucleic acid may encode an antigen for the treatment or prevention of a number of conditions including but not limited to cancer, allergies, toxicity and infection by a pathogen such as, but not limited to, fungus, viruses including Human Papilloma Viruses (HPV), HIV, HSV2/HSV1, influenza virus (types A, B and C), Polio virus, RSV virus, Rhinoviruses, Rotaviruses, Hepatitis A virus, Norwalk Virus Group, Enteroviruses, Astroviruses, Measles virus, Para Influenza virus, Mumps virus, Varicella-Zoster virus, Cytomegalovirus, Epstein-Barr virus, Adenoviruses, Rubella virus, Human T-cell Lymphoma type I virus (HTLV-I), Hepatitis B virus (HBV), Hepatitis C virus (HCV), Hepatitis D virus, Pox virus, Marburg and Ebola; bacteria including M.tuberculosis, Chlamydia, N.gonorrhoeae, Shigella, Salmonella, Vibrio Cholera, Treponema pallidua, Pseudomonas, Bordetella pertussis, Brucella, Franciscella tulorensis, Helicobacter pylori, Leptospria interrogaus, Legionella pnumophila, Yersinia pestis, Streptococcus (types A and B), Pneumococcus, Meningococcus, Hemophilus influenza (type b), Toxoplama gondii, Complybacteriosis, Moraxella catarrhalis, Donovanosis, and Actinomycosis; fungal pathogens including Candidiasis and Aspergillosis; parasitic pathogens including Taenia, Flukes, Roundworms, Amebiasis, Giardiasis, Cryptosporidium, Schitosoma, Pneumocystis carinii, Trichomoniasis and Trichinosis. The nucleic acid my also be used to provide a suitable immune response against numerous veterinary diseases, such as Foot and Mouth diseases, Coronavirus, Pasteurella multocida, Helicobacter, Strongylus vulgaris, Actinobacillus pleuropneumonia, Bovine viral diarrhea virus (BVDV), Klebsiella pneumoniae, E. coli, Bordetella pertussis, Bordetella parapertussis and Bordetella brochiseptica. Thus in one aspect, the nucleic acid constructs of the present invention may find use in a vaccine. Vaccines of the invention may be used tovaccinate against any of the pathogens and conditions mentioned herein. Thus, the invention provides a vaccine composition comprising a nucleic acid construct of the invention or a population of nucleic acid constructs of the invention or coated particles of the invention.

In one preferred instance a nucleic acid construct of the invention may encode an antigen from a member of the adenoviridae (including for instance a human adenovirus), herpesviridae (including for instance HSV-1, HSV-2, EBV, CMV and VZV), papovaviridae (including for instance HPV), pox-viridae (including for instance smallpox and vaccinia), parvoviridae (including for instance parvovirus B19), reoviridae (including for instance a rotavirus), coronaviridae (including for instance SARS), flaviviridae (including for instance yellow fever, West Nile virus, dengue, hepatitis C and tick-borne encephalitis), picornaviridae (including polio, rhinovirus, and hepatitis A), togaviridae (including for instance rubella virus), filoviridae (including for instance Marburg and Ebola), paramyxoviridae (including for instance a parainfluenza virus, respiratory syncitial virus, mumps and measles), rhabdoviridae (including for instance rabies virus), Bunyaviridae (including for instance Hantaan virus), orthomyxoviridae (including for instance influenza A, B and C viruses), retroviridae (including for instance HIV and HTLV) and hepadnaviridae (including for instance hepatitis B). In a further preferred instance the antigen may be from a pathogen responsible for a veterinary disease and in particular may be from a viral pathogen, including, for instance, a Reovirus (such as African Horse sickness or Bluetongue virus) and Herpes viruses (including equine herpes). The antigen may be one from Foot and Mouth Disease virus. In a further preferred instance the antigen may be from a Tick borne encephalitis virus, dengue virus, SARS, West Nile virus and Hantaan virus.

In another preferred case the antigen may be from a retroviradae (e.g. HTLV-I; HTLV-11; or HIV-1 (also known as HTLV-111, LAV, ARV, hTLR, etc.)). In particular from HIV and in particular the isolates HIVIIIb, HIVSF2, HTVLAV, HIVLAI, HIVMN; HIV-1CM235, HIV-1; or HIV-2. In a particularly preferred embodiment, the antigen may be a human immunodeficiency virus (HIV) antigen. Examples of preferred HIV antigens include, for example, gp120, gp 160 gp41, gag antigens such as p24gag and p55gag, as well as proteins derived from the pol, env, tat, vif, rev, nef, vpr, vpu or LTR regions of HIV. In a particularly preferred case the antigen may be HIV gp120 or a portion of HIV gp120. The antigen may be from an immunodeficiency virus, and may, for example, be from SW or a feline immunodeficiency virus.

Thus, the encoded polypeptide may be an antigen, an immunogenic fragment thereof or an immunogenic variant thereof. The fragment or variant may, for instance, have any of the levels of homology, proportion of the length of the original antigen, and functionality specified herein and in particular ability to give rise to an immune response. In some instances, the encoding sequence of the nucleic acid construct may have been modified to optimize expression. For instance, codon usage may be modified to that typical of the subject. A consensus Kozak sequence for the subject may also be substituted for the naturally occurring sequence around the start codon.

In an especially preferred embodiment the nucleic acid construct of the invention comprises a coding sequence encoding an influenza antigen, an immunogenic fragment thereof or an immunogenic variant of either. The fragment and/or variant may have any of the levels of sequence homology, fragment lengths and/or levels of functionality specified herein. In particular, preferably a coding sequence of the construct encodes an influenza virus antigen, an immunogenic fragment of an influenza virus antigen or an immunogenic variant with 80% amino acid homology to any of the preceding.

For instance the influenza antigen may be an influenza NP (nucleoprotein/nucleocapsid protein), HA (hemagglutinin), NA (neuraminidase), M1, M2, PB1, PB2, PA, NS1 and/or NS2 antigens or may be a fragment or variant of such antigens. In a preferred embodiment the encoded antigen may be HA, NA and/or M2 influenza antigen or a fragment or a variant of such antigens. In an especially preferred instance, the encoded antigen may be an HA or an NA antigen or a fragment or variant of such antigens and in particular an HA antigen or a fragment or variant of such an antigen.

Thus, in an especially preferred construct of the invention the encoded antigen is influenza hemaglutinin (HA), an immunogenic fragment thereof or an immunogenic variant with 80% amino acid sequence homology to either. In a further preferred instance the encoded antigen is influenza Neuraminidase (NA), M2, an immunogenic fragment of either or an immunogenic variant with 80% amino acid sequence homology to any of the preceding.

In a preferred instance, a construct of the invention may encode more than one polypeptide and in particular more than one influenza antigen, immunogenic fragment or immunogenic variant of either. In instances where more than one antigen is to be employed in a preferred instance HA and NA antigens may be employed together or a fragment or a variant of such antigens.

In instances where a construct of the invention expresses more than one influenza antigen, immunogenic fragment, or immunogenic variant of either, at least two of the different antigens, fragments or variants encoded are from the same influenza polypeptide from different strains of influenza virus.

In one preferred embodiment the antigen may be from the H5N1 strain of influenza and immunogenic fragments thereof and variants of either which retain immunogenicity may be employed. In particular, the antigen may be one from the H5N1 strain or a fragment of such an antigen.

In some instances, the antigen may be a fragment or variant of a naturally occurring influenza polypeptide. For instance, the antigen may correspond to a sub-region of a naturally occurring influenza polypeptide including any of the various fragment lengths referred to herein. The antigen may be a variant of a naturally occurring influenza antigen or of a fragment of such an antigen. Preferably such variants and/or fragments will be able to give rise to an immune response capable of recognising the antigen and in particular the influenza virus the fragment or variant is derived from.

The influenza antigen may be from any influenza virus. The antigen may be from influenza virus A, B or C, in particular from influenza A and/or B. The antigen may be from a variant influenza strain and in particular a variant strain associated with increased infectivity or pathogenicity of the influenza strain. The antigen may, for instance, be from one of the strains identified annually by the World Health Organisation to be used in influenza vaccines and in particular may be an antigen identified by the WHO for such use. In a preferred instance, a nucleic acid construct, population of nucleic acids, pharmaceutical composition or vaccine of the invention may encode an antigen of each of three influenza strains and in particular the three strains identified by WHO or other equivalent authorities in a particular year.

In a preferred instance, one or more of the encoded influenza antigens may originate from a pandemic influenza strain. Thus, the influenza antigen, immunogenic fragment or variant of either may be from a pandemic influenza strain. A construct encoding the antigen from the pandemic strain may, for instance, be administered, or be present, on its own. In others, the construct may also encode, or be administered with other constructs that encode, other antigens. In a preferred case, an antigen from a pandemic influenza strain and an antigen from a non-pandemic influenza strain may be encoded either on the same or separate constructs. In particular, a pandemic flu antigen and an antigen from 1, 2, 3, 4, 5, 6 or more non-pandemic influenza strains may be administered, preferably an antigen from each of 3, 4 or 5 non-pandemic influenza strains may be administered and even more preferably an antigen from each of 3 or 4 non-pandemic influenza strains may be administered.

In some instances, the construct may encode more than one polypeptide. In particular, a construct may encode more than one antigen and especially more than one influenza antigen. For instance, the construct may express two, three, four, five, six or more polypeptides and in particular antigens. In a preferred case, the construct may encode three, four, five or more different polypeptides and in particular antigens. In an even more preferred case the construct may encode three, four or five different polypeptides and in particular antigens. In a still more preferred case the construct may encode three or four different polypeptides, in particular antigens and especially influenza antigens. At least one of the antigens will be expressed using the chimeric promoter of the invention and preferably all of the antigens will be so expressed. Typically, each antigen may be expressed from a separate chimeric promoter of the invention. In some cases, a single promoter may be used to generate a transcript which gives rise to a plurality of polypeptides. In some instances, several antigens may be expressed as a fusion protein.

In a preferred instance, a construct of the invention encodes a pandemic influenza antigen, immunogenic fragment thereof, or immunogenic variant of either and one or more non-pandemic influenza antigens, immunogenic fragments thereof or immunogenic variants of either.

The nucleic acid constructs of the invention may be employed in a vaccine. Thus, the invention provides a vaccine composition comprising a nucleic acid construct, a population of nucleic acid constructs or coated carrier particles of the invention. The vaccine may comprise a suitable pharmaceutical carrier or excipient. The vaccine may comprise an adjuvant and in particular an adjuvant construct of the invention. Alternatively, the vaccine may be administered separately, sequentially or simultaneously with such an adjuvant.

In one preferred instance, the invention provides a multivalent vaccine comprising at least two different constructs of the invention that encode different antigens, immunogenic fragments thereof, or immunogenic variants of either. The antigen may be any of those mentioned herein. In a preferred instance, the invention provides a multivalent vaccine comprising at least two different constructs of the invention which encode different influenza antigens, immunogenic fragments thereof or immunogenic variants of either. In alternative instances, a plurality of antigens may be expressed from the same construct to provide a multivalent vaccine or combinations of constructs encoding single and plural antigens may be used.

In a further preferred instance, a multivalent vaccine of the invention may be one which is a trivalent, tetravalent or pentavalent vaccine encoding three, four or five different antigens, immunogenic fragments or immunogenic variants and in particular influenza antigens, fragments or variants of either.

In another preferred instance a vaccine of the invention, including the multivalent vaccines, may comprises a construct which encodes a pandemic influenza antigen, immunogenic fragment thereof or immunogenic variant of either. A multivalent vaccine may encode a pandemic influenza antigen, immunogenic fragment, or immunogenic variant of either and also, for instance, an antigen from each of three, four or five different influenza strains, immunogenic fragments thereof or immunogenic variants of either.

An adjuvant may be present in a vaccine of the invention or administered simultaneously, separately or sequentially with a vaccine of the invention. In particular, the invention provides a vaccine which comprises at least one construct of the invention encoding an ADP ribosylating bacterial toxin subunit, fragment thereof with adjuvant activity or variant thereof with adjuvant activity.

Nucleic acid constructs expressing more than one antigen may be used to generate multivalent vaccines, i.e. a vaccine intended to immunize against a plurality of different antigens and in particular against influenza antigens. In some instances, an influenza antigen or antigens and an antigen or antigens from a different pathogen will be provided. Thus, any of the constructs, populations of nucleic acid constructs, vaccines, pharmaceutical compositions and coated carrier particles mentioned herein may comprise a construct encoding an influenza antigen and either the same construct, or different constructs, may encode antigens from different pathogens. The various multivalent influenza constructs, populations and vaccines mentioned herein may also encode an antigen or antigens from a different pathogen.

In some cases, the antigens encoded will be from the same pathogen. In some instances, the different antigens will all be from the same virus. Thus, in a preferred instance, all of the antigens will be influenza antigens. The plurality of antigens may come from the same strain influenza virus and hence may be from several different polypeptides of the influenza virus. Alternatively, the plurality of antigens may be from different strains of virus and in particular influenza virus. In the case of multivalent constructs and/or vaccines antigens from at least two and preferably at least three, four or five different influenza strains may be chosen. The antigens may, for instance, be from 2, 3, 4, 5, 6 or more different strains, in particular antigens from three, four or five strains may be employed, and even more preferably from three or four different strains.

Multivalent vaccines may alternatively or additionally comprise a plurality of different constructs of the invention with the different constructs encoding different antigens. For instance, at least 2, 3, 4, 5, or 6 different constructs may be employed. In a particularly preferred instance 2, 3, 4 or 5 different constructs and especially three or four constructs may be employed. In one instance, where more than one construct is present, each construct encodes a singular antigen. In a further instance, constructs may encode 2, 3, 4, 5 or more different antigens, particularly 3 or 4 different antigens.

The invention also provides a population of nucleic acid constructs where the population comprises a plurality of constructs of the invention and in particular any of the combinations mentioned above. Thus, the invention provides a population of nucleic acid constructs where the population comprises at least two different constructs of the invention. The nucleic acid constructs may be present in any suitable amounts relative to each other. Typically, each nucleic acid construct is present in a weight ratio of approximately 1:10 to each other.

The populations of nucleic acid constructs may be used to generate multivalent vaccines and in the various methods of the invention. Multivalent vaccines may also be multivalent because they comprise any of the constructs of the invention that encode more than one antigen.

In one preferred instance a population of nucleic acids is provided where the population comprises at least two different constructs that encode different antigens. In particular, the invention provides a population comprising at least two different constructs which encode influenza antigens, immunogenic fragments thereof or immunogenic variants of either. In such a population of nucleic acid constructs preferably at least two of the different antigens may be from the same influenza polypeptide, such as HA, from different influenza strains. In populations with at least two different constructs which encode influenza antigens are present, preferably at least two of the different antigens, fragments or variants are from different influenza polypeptides may be from the same or a different influenza strain. Further preferred populations include a population of nucleic acid constructs where the population comprises at least three different constructs which each encode an antigen of a different influenza strain or an immunogenic fragment or immunogenic variant.

In another preferred instance, the invention provides a population of nucleic acid constructs which comprises at least one construct encoding an antigen, immunogenic fragment thereof, or an immunogenic variant of either and at least one construct encoding an ADP ribosylating bacterial toxin subunit, fragment thereof with adjuvant activity or variant thereof with adjuvant activity. The or each construct encoding an antigen, immunogenic fragment or immunogenic variant is typically present at a weight ratio of about 10:1 to 1:10 with respect to the or each construct encoding the ADP ribosylating bacterial toxin subunit, fragment thereof with adjuvant activity or variant thereof with adjuvant activity. The weight ratio may be from about 10:1 to about 1:1, for example about 9:1.

In instances where a plurality of polypeptides is to be encoded, any combination of constructs encoding one or more polypeptides and a plurality of constructs may be employed. For instance, where three antigens are to be encoded, one construct may encode all three, one construct may encode two and another construct one antigen, or three constructs each encoding one antigen may, for instance, be employed. In instances where four antigens are to be encoded, they may be encoded via four, three, two or one construct, with each construct encoding one, two, three, or four different antigens. In one instance, as few as possible constructs may be employed, for instance only one or two constructs may be employed. The invention also provides populations of nucleic acids and vaccines comprising such combinations of nucleic acid constructs.

In a preferred instance, the invention provides a multivalent vaccine or population of nucleic acids comprising a construct encoding an antigen from a pandemic influenza virus and which also encodes 3, 4 or 5 and in particular 3 or 4 non-pandemic influenza antigens. The non-pandemic influenza antigens may be encoded on the same construct as the pandemic influenza antigen or on different constructs. In a preferred instance the 3, 4 or 5 other non-pandemic influenza antigens may be encoded on a separate construct or constructs and in particular on a single separate construct. In another instance, the construct encoding the pandemic antigen may also encode one or more of the other antigens.

In some embodiments, the nucleic acid construct will encode an adjuvant or a separate construct may do so. Any of the populations, compositions and vaccines of the invention may comprise, or may be administered simultaneously, sequentially or separately with such an adjuvant construct. Thus, the sequences inserted into the cloning site for insertion of a coding sequence may encode a polypeptide that can act as an adjuvant.

Thus, in one instance the invention comprises a nucleic acid construct wherein the coding sequence encodes an ADP ribosylating bacterial toxin subunit, a fragment thereof with adjuvant activity or a variant of either with adjuvant activity which also has 80% amino acid homology to any of the preceding. In a preferred instance, the nucleic acid construct comprises two coding sequences comprising an ADP ribosylating bacterial toxin subunit, fragment thereof with adjuvant activity or variant of either with adjuvant activity, where each is linked to such a chimeric promoter.

The two coding sequences encoding an ADP ribosylating bacterial toxin subunit, fragment thereof with adjuvant activity or variant of either with adjuvant activity may be, in one instance, in inverse orientation. In another instance the two coding sequences encoding an ADP ribosylating bacterial toxin subunit, fragment thereof with adjuvant activity or variant of either with adjuvant activity may be in the same orientation. In any of the constructs of the invention comprising more than one coding sequence linked to a chimeric promoter, the promoters may be in the same orientation, in different orientations or a mixture of the two where there are three or more such promoters.

In a preferred instance, the encoded adjuvant in any of the adjuvant constructs may be an ADP-ribosylating bacterial toxin. These include diphtheria toxin (DT), pertussis toxin (PT), cholera toxin (CT), the E. coli heat labile toxins (LT1 and LT2), Pseudomonas endotoxin A, Pseudomonas exotoxin S, B cereus exoenzyme, B. sphaericus toxin, C botulinum C2 and C3 toxins, C. limosum exoenzyme, as well as toxins from C. perfringens, C spiriforma and C. difficile and Staphylococcus aureus EDIN. Most ADP-ribosylating bacterial toxins contain A and B subunits. The construct may express the A subunit, the B subunit and/or both subunits.

In a preferred instance, the nucleic acid construct may encode the E. coli heat labile toxin and/or cholera toxin and in particular may express E. coli heat labile toxin. A GenBank entry for the complete sequences of the CT subunit A and B genes can be found at Locus VIBCTXABB (Accession No. D30053), while a GenBank entry for the complete sequences of the LT subunit A and B genes can be found at locus AB0116677 (Accession No. AB011677). In a particularly preferred instance, a construct of the invention may encode the LT A and/or LT B subunits encoded by the vectors pPJV2012 and/or pPJV7788 or a fragment of such a subunit which retains adjuvant activity or a variant of either which retains adjuvant activity. A construct of the invention may comprise the coding sequences for the LT A and/or LTB subunits of the vectors pPJV2012 and/or pPJV7788, a fragment thereof which encodes a polypeptide which has adjuvant activity or a variant of either which has adjuvant activity. In a preferred instance a construct of the invention has both the LT A and LT B coding sequences.

In another preferred instance, the construct may comprise the sequence of the vector PJV2012 provided by SEQ ID No: 61 or a sequence with 60% sequence identity thereto. In a further instance, the construct may comprise the sequence of the vector PJV7788 provided by SEQ ID No: 62 or a sequence with 60% sequence identity thereto.

The construct may express an active variant or fragment of a particular adjuvant. The variant or fragment will be said to be active if it retains at least some of the adjuvant activity of the polypeptide it is derived from. Thus, the variant and/or fragment will still be able to enhance an immune response against a particular antigen in comparison to the immune response seen when no adjuvant is administered with the antigen. The encoded sequence may be active fragments or variants of CT A and/or B subunits and in particular may be active fragments of LT A and/or B subunits. Variants and fragments which may be employed may, for instance, have any of the lengths, levels of sequence homology or other characteristics mentioned herein for variants and fragments. They may, for instance, be assessed using the Comparative Immunogenicity Assay using a particular antigen and then comparing the results seen with an adjuvant base vector and a variant vector encoding an adjuvant. In a particularly preferred embodiment the construct may encode the LTA and/or LTB subunits and in particular both. The construct may, in a particularly preferred instance be pPJV2012 or pPJV7788 the sequences for which are provided herein.

Any of the adjuvant constructs of the invention may be administered simultaneously, sequentially or separately with an antigen or a nucleic acid encoding an antigen. In a preferred instance an adjuvant construct of the invention may be administered simultaneously, sequentially or separately with a construct of the invention encoding an antigen. Compositions and vaccines comprising an adjuvant construct and a construct encoding an antigen are provided, as are core carriers coated with both types of construct provided on them or mixtures of core carriers coated with an adjuvant construct and other core carriers coated with a construct encoding an antigen.

The toxin subunit may have had its naturally occurring signal sequence deleted. The natural signal sequences of the exotoxins may be replaced by a eukaryotic signal sequence and in particular by chicken lysozyme signal peptide. A naturally occurring exotoxin subunit may have been modified to detoxify the toxin. The A subunit may have been modified to disrupt or inactivate ADP-ribosyl transferase activity. In some cases the exotoxin subunits may retain toxicity. Thus, in some instances the exotoxin subunits may not have been detoxified.

Thus, the adjuvant constructs of the invention may be used to enhance an immune response against a particular antigen. The enhanced immune response may involve an immune response of greater magnitude or duration. In may mean that when the antigen is re-encountered the immune response then is greater than if no adjuvant was administered. The enhanced immune response may result in higher antibody titres. In the case of some constructs, and in particular those expressing exotoxin subunits, the adjuvant may result in an augmented cellular response and a T helper 1-like immune response against the antigen in question.

The adjuvant constructs may be administered with, be present in a vaccine with, or be present in a population of nucleic acids with any of the other constructs mentioned herein. The adjuvant constructs may also encode, or be administered with a construct which encodes, an antigen or antigen, including any of these mentioned herein and in particular those encoding influenza.

In one embodiment, a construct of the invention may encode an immunostimulator and/or an immunosuppressor. In a preferred instance a construct may encode one or more of interferon alpha, beta and/or gamma, interleukin-1, -2, -4, -5, -7, -10, -12, -13, -18, -23 and -24, GM-CSF, G-CSF, TGF-beta, B7.1, B7.2, CTLA-4, CD40 ligand, CD40, OX40, OX40 ligand, Flt-3 ligand, TRAIL, TRANCE, Fas ligand, TNF alpha, MCP-1 alpha, PF-4, SLC, MIP-3 alpha, IP-10. In some cases such a construct may be administered simultaneously, separately or sequentially with any other construct, in particular one of the invention and especially a construct encoding an antigen.

A construct of the invention may comprise a polyadenylation signal. The nucleic acid for insertion into the cloning site may comprise a polyadenylation (polyA) sequence. Such a polyA sequence is generally native to the coding sequence. Alternatively, a heterologous polyA sequence may be provided in the nucleic acid construct of the invention. Typically the polyA sequence will be provided downstream of the cloning site, such that it is operatively linked to a coding sequence inserted in the cloning site. Any suitable polyA sequence may be included in the construct using standard cloning techniques. Such polyA sequences are known in the art.

The poly A sequence may be:
(i) a natural poly A sequence;
(ii) a functional homologous variant of (i); or
(iii) a functional fragment of (i) or (ii).

The natural poly A sequence (i) may be, for example a rabbit, beta globin gene poly A, Human Papilloma Virus (HPV) early or late gene poly A, HSV-2gB gene poly A, a simian CMV immediate early gene poly A or HSV gD late gene poly A. The polyadenylation sequence may be derived from a polyadenylation sequence of a gene selected from that of the rabbit beta-globin gene, human papilloma virus (HPV) early or late genes, the HSV-2gB gene, a simian CMV immediate early gene and HSVgD late gene.

Preferably the natural poly A sequence (i) is selected from the group consisting of SEQ ID No. 10 (GenBank K03256), SEQ ID No. 11 (GenBank M16019), SEQ ID No. 12 (GenBank 280699) and SEQ ID No. 13 (GenBank K02718). A particularly preferred poly A sequence comprises nucleotides 4243 to 4373 of SEQ ID NO: 54 or is a functional homologous variant or fragment thereof. A preferred poly A sequence is also provided by nucleotides 2556 to 2686 of SEQ ID No. 14 or a functional fragment or variant of either may also be employed.

In a further preferred instance, a polyadenylation signal employed in a construct of the invention may comprise:
(i) the nucleotide sequence of SEQ ID NO:10, SEQ ID NO:11, SEQ ID NO:12, SEQ ID NO:13, nucleotides 4243 to 4373 of SEQ ID No:54, nucleotides 906 to 1038 of SEQ ID No: 61, nucleotides 4375 to 4050 of SEQ ID No:61, and/or nucleotides 2463 to 2593 of SEQ ID No:62;
(ii) a functional variant of (i) which has at least 80% nucleotide sequence homology to one or more of the sequences of (i); and/or
(iii) a functional fragment of (i) or (ii).

In general, a functional polyA sequence is one which retains polyadenylation activity.

A poly A sequence may be tested for the ability to bring about polyadenylation of an RNA transcript using a routine expression assay. A functional homologous variant (ii) or functional fragment (iii) typically shows at least 50%, for example 60%, 70% 80% or more of the poly A activity of the natural poly A sequence in the assay.

Generally the poly A sequence will, when present in the construct of the invention, enhance expression, typically causing comparable enhancement to the natural poly A sequence (i) mentioned above.

A poly A sequence may also be assayed for functionality using the Comparative Expression Assay below. A test poly A region is swapped into the base vector in place of the RBGpA. A test poly A is considered functional if the poly A does not abrogate expression, but preferably increases expression in at least one but preferably two reference cell types, compared to the base vector. Preferably there is an increase in expression of at least 5%, 10%, 20%, 30%, 40% or 50% or more. The increase may be any of the levels mentioned herein. In some cases a decrease may occur, including any of the levels mentioned herein. Preferred cell types are mammalian HEK293T, CHO, HeLa, BHK, 3T3 or COS cells. SSC15 or B16 cells may also be employed. According to the assay, typically an homologous variant (ii) or fragment (iii) is functional if it allows greater than 50% of the expression improvement achieved by the natural poly A sequence (i).

Alternatively, or additionally, poly A sequences may be tested for activity in the Comparative Immunogenicity Assay below. Poly A sequence is swapped into the base vector in place of RBGpA. A functional poly A sequence provides antibody titres that are at least as high as or higher than those achieved by the base vector with at least one, preferably two antigens. Preferably the antibody titres are at least 5%, 10%, for instance 20%, 30% or 40% higher than those achieved with the base vector. In some instances any of the increases or decreases mentioned herein may be seen. Preferred antigens are HBsAg, HSV2gD and Flu-M2 antigens. Especially preferred antigens are influenza antigens, including any of those mentioned herein and in particular HA, NA and M2 antigens. In particular, HA antigen, or an immunogenic fragment thereof or an immunogenic variant of either may be employed. An homologous variant (ii) or fragment (iii) is typically functional if it allows the highest antibody titres achieved by the natural poly A sequence (i).

The nucleic acid construct may comprise additional control sequences which influence expression of a coding sequence inserted in the cloning site. The construct may include a non-translated leader sequence. The sequence is provided in the construct in operable linkage with the chimeric promoter, and therefore also with a coding sequence inserted at the cloning site. The leader provides a translational start site for expression of an inserted coding sequence and typically includes a Kozak sequence.

Typically the untranslated leader sequence comprises:
(i) a natural untranslated leader sequence;
(ii) a functional homologous variant of (i); or
(iii) a functional fragment of (i) or (ii).

Typically the leader sequence comprises sequences present in (i) which bind transcription components or regulatory proteins, or homologues of these sequences which are able to bind the same components or proteins. Typically such sequences or their homologues are present in the leader sequence in the same order and/or substantially the same relative spacing as in (i). In general the leader sequence comprises a translational start site for expression of an inserted coding sequence. Typically the leader sequence includes a Kozak sequence.

In general the untranslated leader sequence has a length of from about 10 to about 200 nucleotides, for example from about 15 to 150 nucleotides, preferably 15 to about 130 nucleotides. Leader sequences comprising, for example, 15, 50, 75 or 100 nucleotides may be used.

Generally a functional untranslated leader sequence is one which is able to provide a translational start site for expression of a coding sequence in operable linkage with the leader sequence. Typically a functional variant (ii) or fragment (iii) has substantially the same activity as and/or complements the activity of the natural sequence (i), usually in facilitating or enhancing expression of a coding sequence in operable linkage with the sequence.

A variant (ii) or fragment (iii) may be tested for activity as an untranslated leader sequence relative to natural leader sequence using standard protocols. For example, expression vectors may be prepared comprising a natural leader sequence (i) operably linked to its native coding sequence, and expression monitored in suitable host cells e.g. mammalian HEK 293T, CHO, HeLa, BHK, 3T3 or COS cells. Test constructs may be prepared in which the natural leader sequence is replaced by an homologous variant or fragment and expression is monitored again in the same host cells. In general, a variant (ii) or fragment (iii) provides at least 50%, such as 60%, 70%, 80%, 90% or 100% or more of the expression provided by the natural sequence. In some cases any of the increases or decreases in expression mentioned herein may be seen.

A potential leader sequence can also be tested for utility in the Comparative Expression Assay below. A test leader sequence is swapped into the base vector in place of the HBV pre S2 5′UTR. A functional leader sequence does not abrogate expression but preferably increases expression in at least one but preferably two reference cell types, compared to the base vector. In general expression is increased by at least 5%, 10%, 20%, 30%, 40% or 50%. Preferred cell types are mammalian HEK293T, CHO, HeLa, BHK, 3T3 or COS cells. According to the assay, an homologous variant (ii) or fragment (iii) is typically functional if it allows greater than 50% of the expression improvement achieved by the natural leader sequence.

Alternatively, or additionally, a leader sequence may be tested for activity in the Comparative Immunogenicity Assay below. A leader sequence is swapped into the base vector in place of HBV pre S2 5′ UTR. A functional leader sequence provides antibody titres that are at least as high as or higher than those achieved by the base vector, with at least one preferably two antigens. Preferably the antibody titres are at least 5%, 10%, 20%, 30% or 40% higher than with the base vector. Preferred antigens are HBsAg, HSV 2gD and Flu-M2 antigens. Especially preferred antigens are influenza antigens, including any of those mentioned herein and in particular HA, NA and M2 antigens. In particular, HA antigen, or a fragment or variant thereof may be employed. An homologous variant (ii) or fragment (iii) is typically functional if it allows the highest antibody titres allowed by the natural leader sequence (i). However, in some instances one of the decreased levels of expression mentioned herein may be seen.

Suitable leader sequence can be obtained using standard protocols. For example, HBV preS2 antigen sequence, HBV e-antigen sequence and HSV type 2 gD antigen sequence is available at GenBank M54923, M54923 and 286099 respectively. Primers can be designed based on this known sequence and used to isolate homologous sequences. Leader sequences may be synthesised based on known sequences.

In general the natural sequence (i) is a eukaryotic sequence or a viral sequence, in particular, of a virus which infects a eukaryote. Preferably the natural sequence (i) is HBV or HSV sequence, for example HBV preS2 antigen sequence, HBV e-antigen sequence, or HSV type 2 gD antigen sequence. Particularly preferably, (i) is selected from the group consisting of SEQ ID No. 5, SEQ ID No. 6 and SEQ ID No. 7. In a further preferred instance, the untranslated leader sequence comprises nucleotides 1864-1984 of SEQ ID NO: 54 or SEQ ID No. 14. Functional fragments or variants of any of these sequences may also be employed.

In one instance, the non-translated leader sequence comprises:
(i) the nucleotide sequence of SEQ ID No 5, Seq ID No: 6, Seq ID No: 7 or nucleotides 1864 to 1984 of SEQ ID No. 54.
(ii) a functional variant of (i) which has at least 80% nucleotide sequence homology to (i); and/or
(iii) a functional fragment of (i) or (ii).

Such a non-translated leader sequence may be employed in any of the constructs of the invention.

The nucleic acid construct may comprise an enhancer sequence. An enhancer sequence is typically provided 3′ of the cloning site, in operable linkage with both the chimeric promoter and an inserted coding sequence, and acts to increase transcription of the inserted sequence.

In general the enhancer comprises:
(i) a natural enhancer;
(ii) a functional homologous variant of (i); or
(iii) a functional fragment of (i) or (ii).

The enhancer sequence generally comprises from about 50 to about 850 nucleotides, for example from about 75 to about 500 nucleotides. Enhancers of about 100, 200, 300 or 400 nucleotides may be used.

Typically (i) is a eukaryotic or viral enhancer, in particular, of a virus which infects eukaryotes. Usually such enhancers occur in the 3′ untranslated region (3′ UTR) of a gene. Preferably (i) is an HBV or a CMV enhancer, for example an HBs Ag 3′UTR or a simian CMV immediate early gene 3′ UTR. Preferably (i) comprises SEQ ID No. 8 or SEQ ID No. 9. Preferably (i) may comprise nucleotides 3699-4231 of SEQ ID NO: 54. In another preferred instance (i) may comprise nucleotides 2012 to 2544 of SEQ ID No. 14. Functional fragments or variants of any of these sequences may be employed.

In general, the enhancer in the construct comprises sequences found in (i) which bind transcription components or regulatory proteins, for example transcription factors, or homologues of these sequences which bind the same components or proteins. Preferably these sequences are present in the enhancer in the same order and/or substantially the same relative spacing as in (i).

Generally a functional enhancer is one which enhances or increases expression of a polynucleotide, for example, a coding sequence, which is operably linked to the enhancer sequence. Typically a functional homologous variant (ii) or fragment (iii) has substantially the same activity (for example, enhancement of expression) as and/or complements the activity of the natural enhancer (i).

Enhancer activity may be assayed using an enhancer trap assay. Protocols are known in the art. A functional homologous variant (ii) or fragment (iii) preferably provides at least 50% of the enhancer activity shown by the natural enhancer in such as assay. Typically the activity is at least 60%, 70%, 80%, 90, 100% or more of the activity of the natural enhancer. In general, a functional variant (ii) or fragment (iii) is able to complement the activity of the natural enhancer (i) in the assay. Any of the increases or decreases referred to herein may, for instance, be seen.

In one preferred instance, the enhancer sequence comprises:
(i) the nucleotide sequence of SEQ ID No:8, SEQ ID No: 9, nucleotides 3699 to 4231 of SEQ ID No. 54, nucleotides 3831 to 4363 of SEQ ID No: 61 and/or 4507 to 5038 of SEQ ID No: 62.
(ii) a functional variant of (i) which has at least 80% nucleotide sequence homology to one or more of the sequences of (i); and/or
(iii) a functional fragment of (i) or (ii).

Enhancer utility may also be tested using the Comparative Expression Assay set out below. A test 3′ UTR sequence is swapped into the base vector. A 3′ UTR has utility if it does not abrogate expression, but preferably increases expression in at least one but preferably two reference cell types compared to the base vectors in the assay. Preferably expression is increased by at least 5%, 10%, 20%, 30%, 40% or 50%. Preferred cell types are mammalian HEK293T, CHO, HeLa, BHK, 3T3 or COS cells. According to this assay, an homologous variant (ii) or fragment (iii) is typically functional if it allows greater than 50% of the expression improvement achieved by the natural enhancer sequence (i).

Additionally or alternatively, enhancer sequences may be tested for activity in the Comparative Immunogenicity Assay below. A 3′ UTR is swapped into the base vector. A functional enhancer sequence provides antibody titres that are at least as high as or higher than those achieved by the base vector with at least one, preferably two antigens. Preferably the antibody titres are at least 5%, 10%, 20%, 30% or 40% higher than with the base vector. Preferred antigens are HBs Ag, HSV2gD and Flu-M2 antigens. Especially preferred antigens are influenza antigens, including any of those mentioned herein and in particular HA, NA and M2 antigens. In particular, HA may be employed or a fragment or variant thereof. An homologous variant (ii) or fragment (iii) is functional if it allows the highest antibody titre allowed by the natural enhancer sequence (i).

Suitable enhancer sequence can be obtained using standard cloning methods. For example, HBsAg 3′ UTR sequence, or simian CMV immediate early gene 3′ UTR sequence can be accessed at GenBank AF143308 and M16019. Primers can be designed based on this known sequence and used to isolate homologous sequences.

In some instances, where a plurality of polypeptides are encoded by a single construct, it may be desirable to omit particular sequences to reduce the size of the construct. In particular, a construct may lack an enhancer and/or region encoding an untranslated leader sequence operably linked to the sequence or sequences encoding the polypeptide to be expressed. This may, for instance, be the case where three, four, five or more polypeptides are expressed from the same construct and particularly three, four or five polypeptides are expressed from the same construct.

In a preferred embodiment, the nucleic acid construct comprises a heterologous polyA sequence, a heterologous leader sequence and a heterologous enhancer, all in operable linkage with the chimeric promoter, for efficient expression of an inserted coding sequence.

In a further aspect, the present invention also provides a nucleic acid construct comprising, or sometimes consisting essentially of:
(i) a promoter sequence
(ii) a non-translated leader sequence derived from HBV preS2 antigen sequence, HBV e-antigen sequence or HSV type 2 gD antigen sequence; and
(iii) a coding sequence operably linked to (i) and (ii)

wherein the coding sequence is heterologous to the non-translated leader sequence.

Typically the promoter sequence (i) is derived from a viral or eukaryotic promoter sequence. The promoter sequence may be a natural promoter sequence, a functional homologue of the natural sequence or a functional fragment of either. Suitable natural promoters include, for example, the hCMV immediate early promoter, Pseudorabies virus (PRV) promoter or Rous sarcoma virus (RSV) promoter. Preferably the natural promoter comprises SEQ ID NO: 52 or SEQ ID NO: 53.

An artificial promoter construct, such as the chimeric promoter described above, may be used, provided that the promoter is functional.

A functional promoter sequence is generally one which is able to cause (including initiate and regulate) transcription of an operably linked coding sequence in a suitable host cell.

A promoter sequence may be tested for promoter activity using a routine expression assay. A functional homologue or fragment of a natural promoter sequence typically provides at least 50%, for example, or least 60, 70, 80 or 90% of the expression provided by the natural sequence in such an assay.

The non-translated leader sequence (ii) is as described above. Suitable coding sequences (iii) include those already described in relation to the chimeric promoter construct. However, in the present aspect of the invention, the coding sequence is heterologous to the non-translated leader sequence. The present construct typically includes a poly A sequence, which as already described, may be native to the coding sequence, or provided as a heterologous poly A sequence in the construct. Suitable poly A sequences have already been described. The construct may additionally include an enhancer sequence 3′ of the coding sequence. Suitable enhancer sequences are described above in relation to the chimeric promoter construct.

In another aspect, the invention provides a nucleic acid construct comprising, or in some embodiments consisting essentially of:
(i) a promoter sequence;
(ii) a coding sequence operably linked to the promoter sequence (i) and;
(iii) an enhancer sequence 3′ of and operably linked to the coding sequence (ii);

wherein the enhancer sequence (iii) is derived from a 3′ UTR of an HBsAg sequence or a 3′ UTR of a simian CMV immediate early gene sequence, and the coding sequence (ii) is heterologous to the enhancer sequence.

The construct may include a non-translated leader sequence such as the ones already described in relation to the chimeric promoter construct.

Typically the promoter sequence (i) is derived from a viral or eukaryotic promoter sequence. The promoter sequence may be a natural promoter sequence, a functional homologue of the natural sequence or a functional fragment of either. Suitable natural promoters include, for example, the hCMV immediate early promoter, Pseudorabies virus (PRV) promoter or Rous sarcoma virus (RSV) promoter. Preferably the natural promoter comprises SEQ ID NO: 52 or SEQ ID NO: 53.

An artificial promoter construct, such as the chimeric promoter described above, may be used, provided that the promoter is functional.

A functional promoter sequence is generally one which is able to cause (including initiate and regulate) transcription of an operably linked coding sequence in a suitable host cell.

A promoter sequence may be tested for promoter activity using a routine expression assay. Functional homologues or fragments of a natural promoter sequence typically provide at least 50%, for example, or least 60, 70, 80 or 90% or the expression provided by the natural sequence in such an assay.

Suitable coding sequences (ii) include those already mentioned in relation to the chimeric promoter construct. However, in the present aspect, the coding sequence is heterologous to the 3′ enhancer sequence. The enhancer sequence (iii) of the construct is described above. The present construct also typically includes a poly A sequence. As in the case of the chimeric promoter construct, this poly A region may be native to the coding sequence (ii) or may be provided as a heterologous poly A component in the construct.

A construct according to any aspect of the present invention may comprise a signal peptide sequence. Thus, a nucleic acid construct may comprise a nucleotide sequence encoding a signal peptide that is operably linked to the coding sequence. The signal peptide sequence is inserted in operable linkage with the promoter such that the signal peptide is expressed and facilitates secretion of a polypeptide encoded by coding sequence also in operable linkage with the promoter.

Typically a signal peptide sequence encodes a peptide of 10 to 30 amino acids for example 15 to 20 amino acids. Often the amino acids are predominantly hydrophobic. In a typical situation, a signal peptide targets a growing polypeptide chain bearing the signal peptide to the endoplasmic reticulum of the expressing cell. The signal peptide is cleaved off in the endoplasmic reticulum, allow for secretion of the polypeptide via the Golgi apparatus.

A signal peptide for use in the invention may comprise:
(i) a natural signal peptide sequence;
(ii) a homologous variant of (i) which retains signal peptide activity; or
(iii) a fragment of (i) or (ii) which retains signal peptide activity.

Sequence (i) may be for example human tissue plasminogen activator signal peptide (hTPAsp) (GenBank L00141), the aprotinin signal peptide (GenBank AAD13685), tobacco extensin signal peptide (GenBank JU0465), or chicken lysozyme signal peptide (GenBank AF410481).

A signal peptide, suitable for use in the present invention, is one which will enable the secretion of heterologous proteins. A functional signal peptide can be identified in an assay which compares the effect of a test signal peptide with the effect of a known signal peptide—e.g. human tissue plasminogen activator signal peptide (hTPAsP)—and with the effect of having no signal peptide. The Comparative Expression Assay set out below may be used but with the following modification. Secretion expression vectors are constructed containing the base vector with either the test signal peptide, hTPAsp or no signal peptide. Coding sequences for polypeptides devoid of their naturally occurring signal peptides are inserted into the vectors and the vectors transformed into reference host cells. Preferably cells are mammalian HEK293T, CHO, HeLa, BHK, 3T3 or COS cells. The cell media is analysed for polypeptide expression levels. A functional signal peptide enables polypeptide secretion at a higher level than a vector lacking a signal peptide with at least one, preferably two polypeptides. Typically, secretion is 5% higher, or more preferably 10% higher or more, for example 20 or 50% higher or more. Typically, secretion levels are comparable to those obtained using hTPAsp. In some cases, any of increases or decreases referred to herein may be seen.

In a preferred instance, the signal peptide is selected from the human tissue plasminogen activator signal peptide (hTPAsp), aprotinin signal peptide, tobacco extension signal peptide and chicken lysozyme signal peptide.

Allowing secretion of encoded protein outside of an expressing cell may have a number of advantages, in particular where the protein is an antigen. For example, increased antigen secretion could allow greater antigen uptake and response by immune cells (macrophages, Langerhan's cells, B-cells, T-cells etc), enable the ability of antigen to reach the bloodstream and signal cells (cytokines), enable an antigen to find cellular ligands and effect a function (antibodies, toxins such as cholera toxin, E. coli LT) and participate in normal cellular biochemical processes (cellular receptors).

A construct of the invention may be in the form of a plasmid. A nucleic acid construct of the invention may be in the form of a plasmid expression vector. In a preferred instance, a construct of the invention may be a DNA construct. In alternative instances, the construct may be an RNA or PNA construct

The vector may then include additional elements, such as an origin of replication, or selector genes. Such elements are known in the art and can be included using standard techniques. In one embodiment, the plasmid vector has the sequence in SEQ ID NO:14. Alternatively, the construct may be included in a viral vector construct.

In some embodiments, the nucleic acid construct of the invention may comprise two or more of the chimeric promoters defined herein. Thus, the construct may comprise a plurality of chimeric promoters and in particular the construct may have two, three, four, five or more chimeric promoters. The chimeric promoters will preferably be each separately operably linked to a cloning site for insertion of a coding sequence. Thus, the construct may express two, three, four, five or more coding sequences. The coding sequences expressed may be any of those specified herein. In a preferred instance, the construct has two chimeric promoters with each having a coding sequence operably linked to them. In particular, the two promoters may be transcribed away from each other. In other embodiments, the promoters may be transcribed towards each other. Thus, the promoters may be in inverted orientation or in some cases the same orientation.

In particular, the constructs with two promoters may express the A and B subunits of an ADP-ribosylating bacterial toxin, including any of those mentioned herein and preferably an LTA and B subunit. However, constructs with multiple promoters may express any combination of the coding sequences mentioned herein. In a further preferred aspect, constructs with multiple promoters may express a plurality of influenza antigens, including combinations of any of the influenza antigens mentioned herein. In another preferred aspect, a construct may express a pandemic influenza antigen, an immunogenic fragment thereof or an immunogenic variant of either and one or more of any of the coding sequences mentioned herein.

In cases where the construct has multiple chimeric promoters each may comprise, or be operably linked to, any of the sequences mentioned herein. In a particularly preferred instance, the heterologous intron of one or more of the promoters may be the rat insulin gene intron A sequence. One or more of the chimeric promoters may also preferably comprise the 5′ UTR of HBVpre-S2. One or more of the promoters may comprise the poly adenylation sequence of the rabbit beta globin gene.

In a preferred case, the nucleic acid construct of the invention may comprise two chimeric promoter sequences, with each promoter sequence being operably linked to a cloning site which has a coding sequence inserted into it, where each chimeric promoter comprises:
(a) a hCMV immediate early promoter sequence;
(b) exon 1 and at least a part of exon 2 of the hCMV major immediate early gene; and
(c) a heterologous intron provided in place of the intron A region of the hCMV major immediate early gene.

with the coding sequence operably linked to one chimeric promoter encoding a LTA subunit and the coding sequence linked to the other encoding an LTB subunit. The construct can therefore express both subunits. Preferably:
the heterologous intron of each promoter is the rat insulin gene intron A sequence;
the sequence encoding each LT subunit is operably linked to the 5′ UTR of HBV pre-S2; and/or
an LT encoding sequence is operably linked to the rabbit beta globin gene polyadenylation sequence.

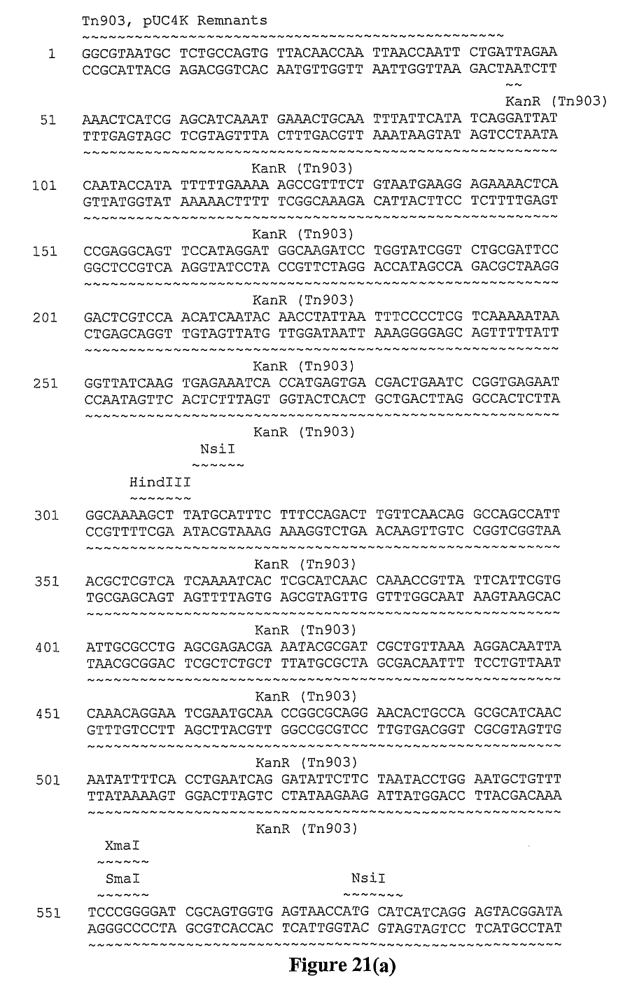

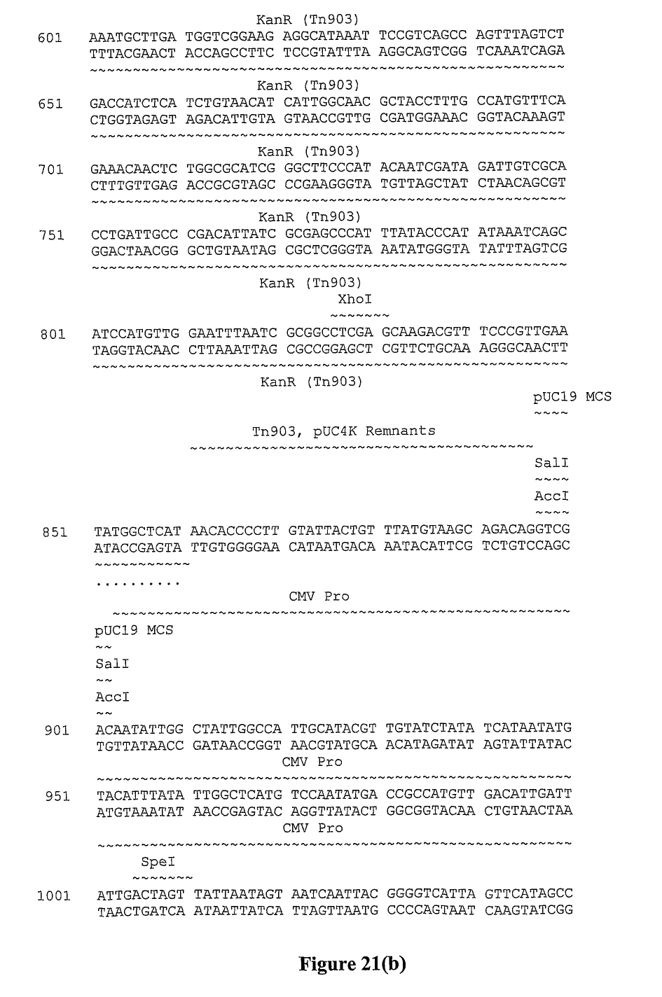

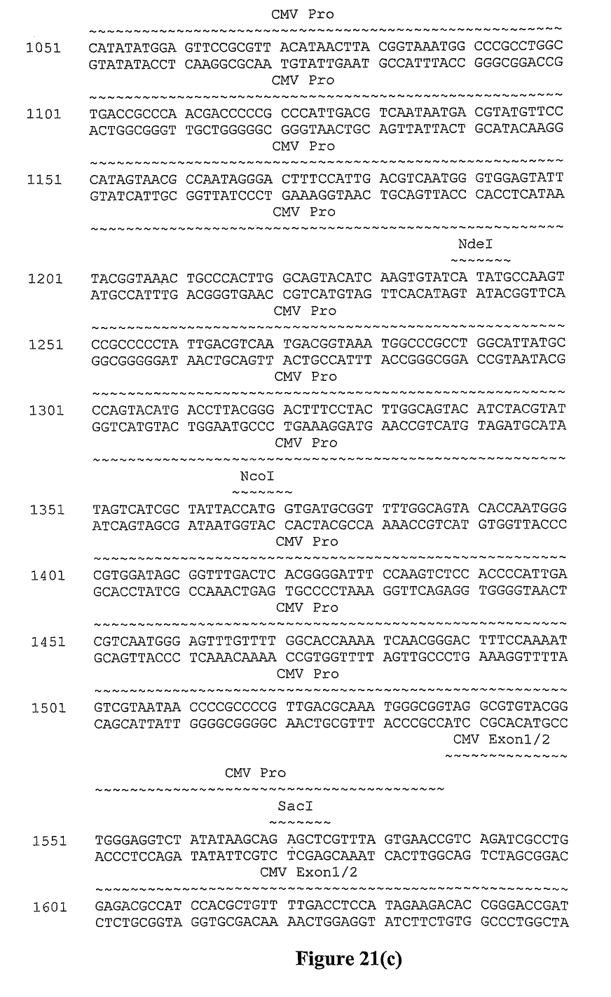

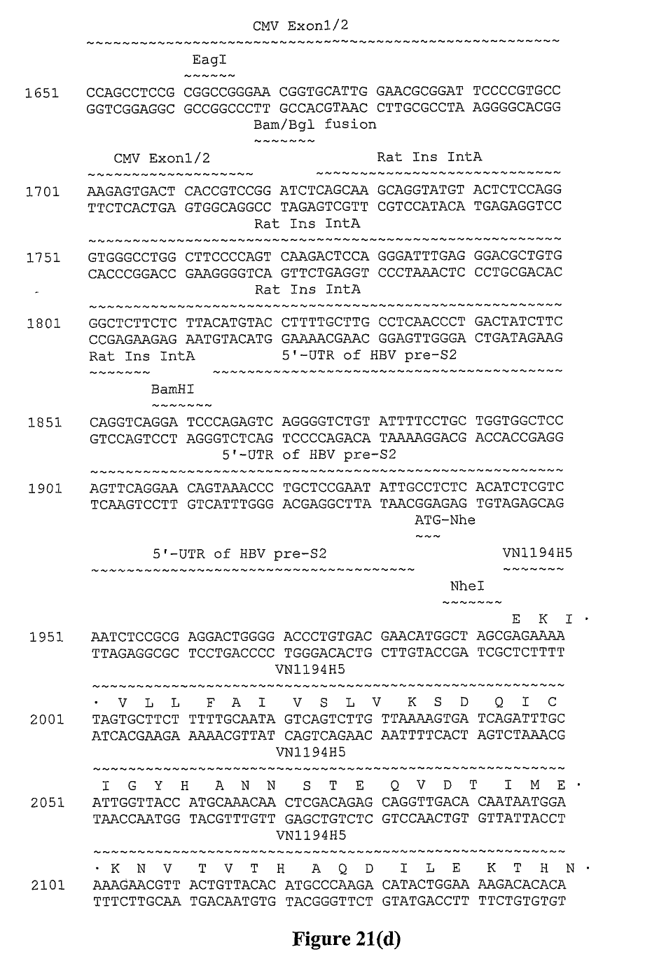

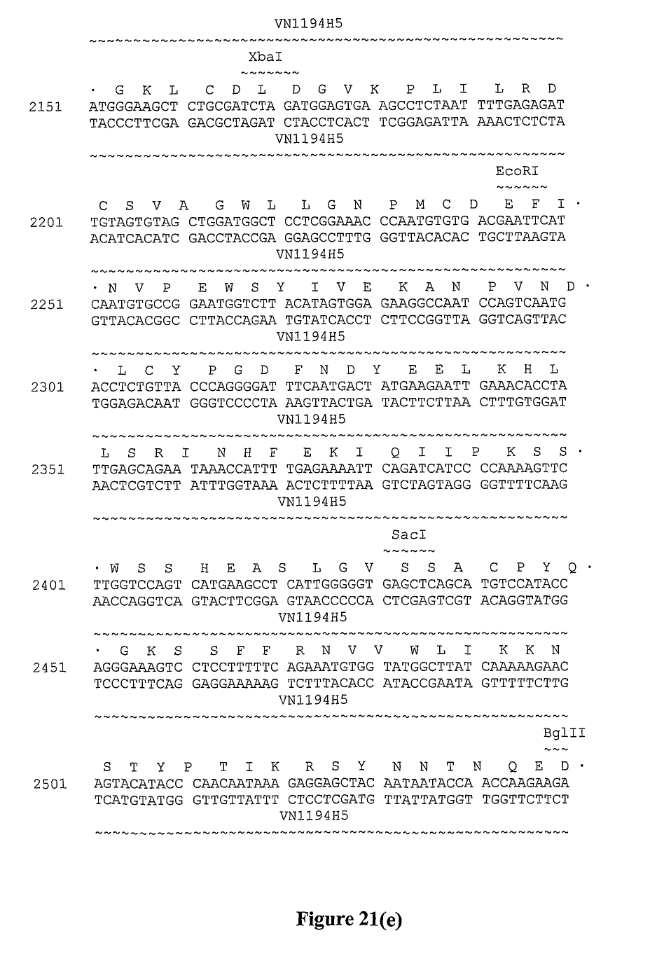

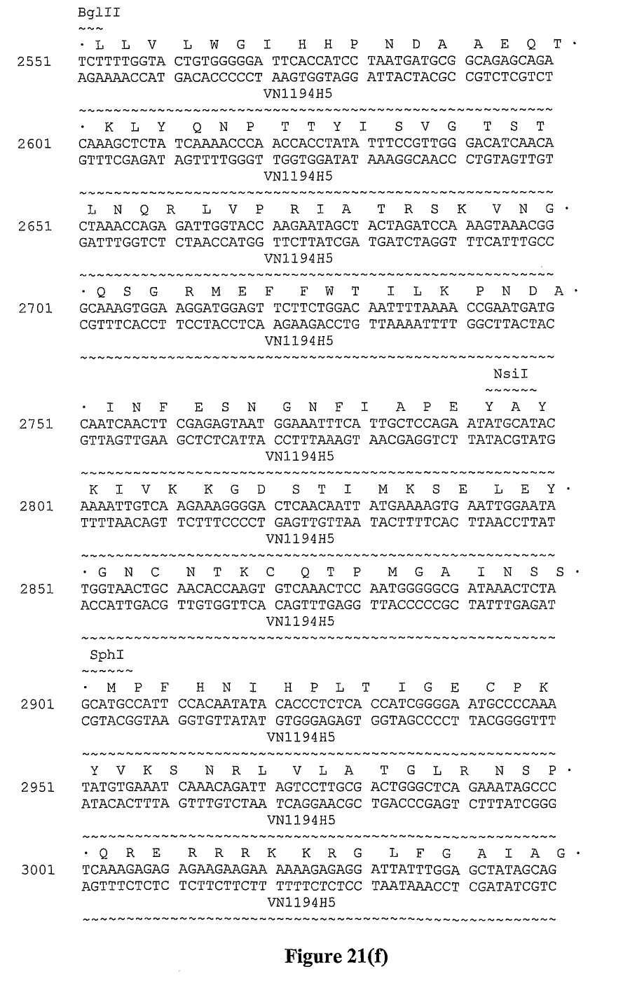

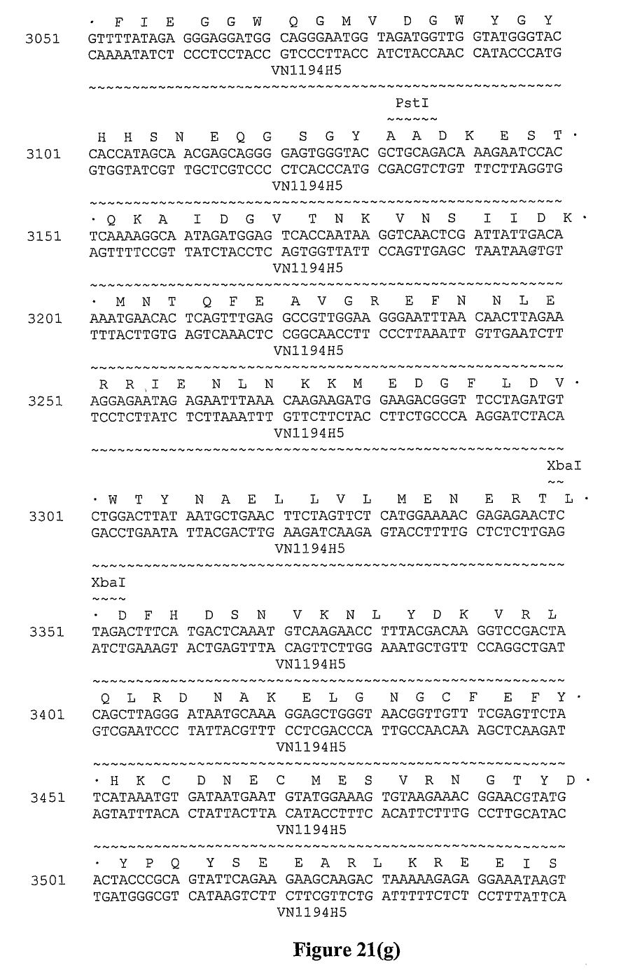

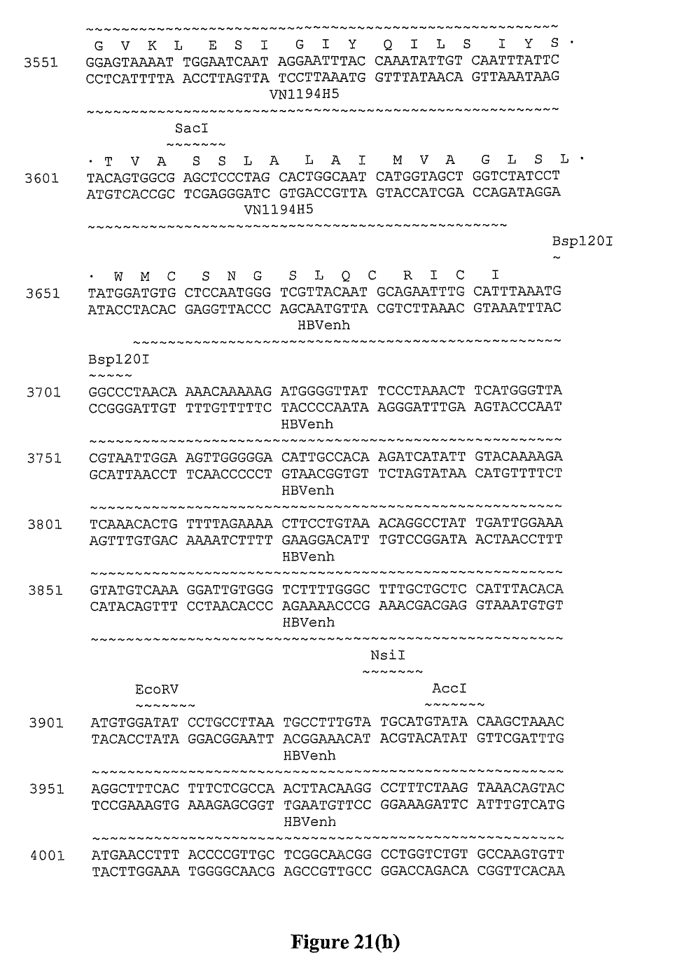

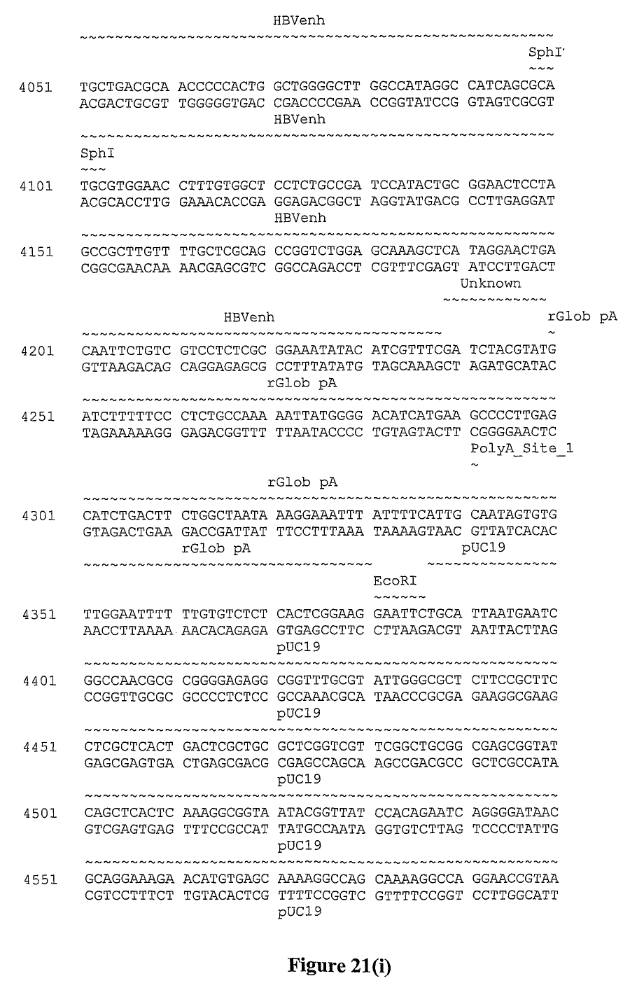

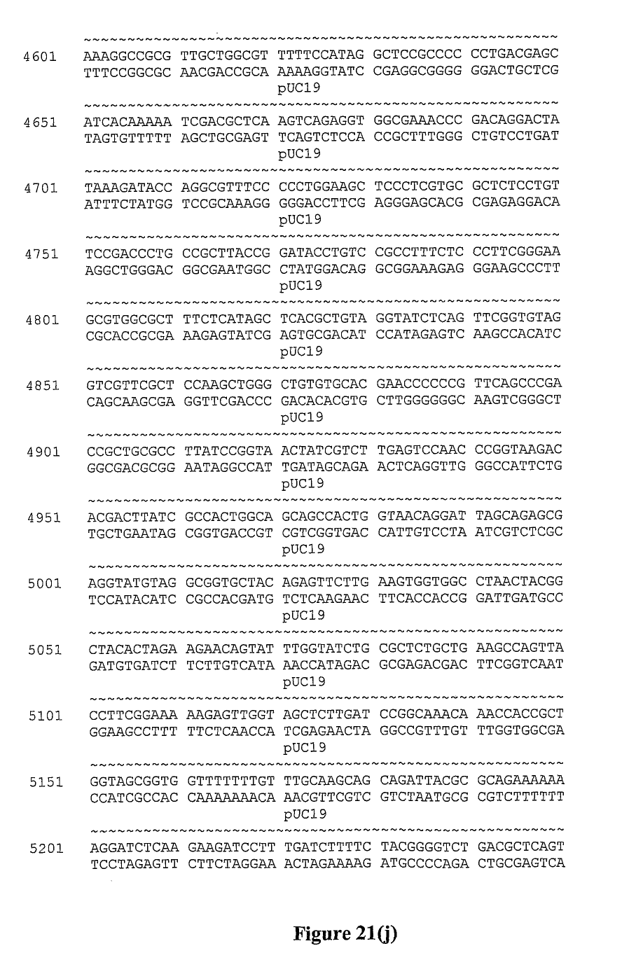

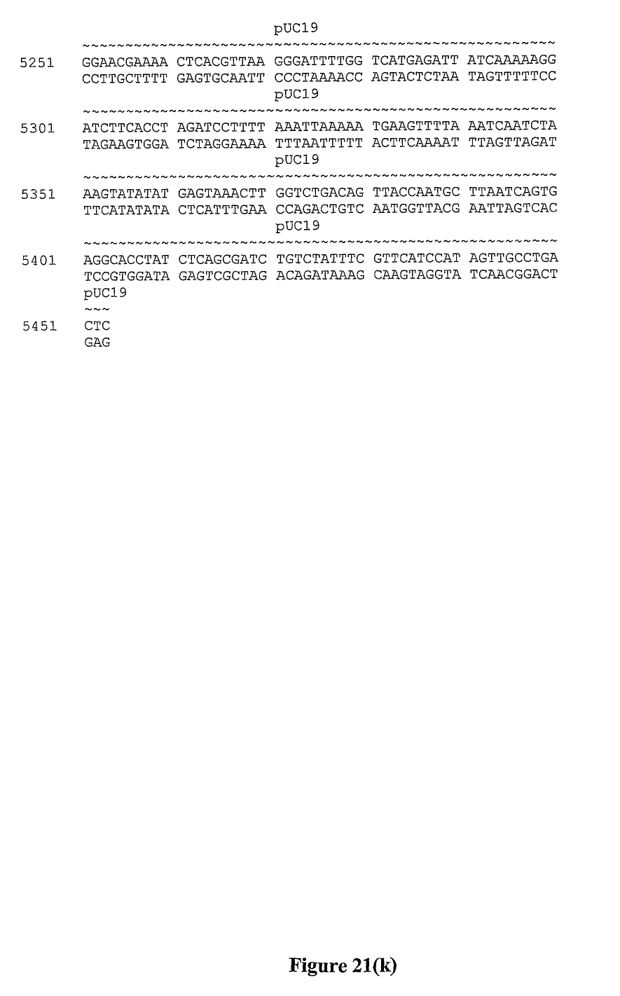

In an especially preferred instance, one or more of the elements of the vectors pPJV7563 and/or pPJV1671 may be employed in a nucleic acid construct of the invention. Functional fragments or variants of such elements may be employed. In particular, any of the elements of pPJV1671 specified in Table 3, functional variants of such elements or functional fragments of either may be employed. Similarly, any of the elements of pPJV7563 specified in Example 5, functional variants of such elements or functional fragments of either may be employed. Preferably, the sequences of one or more of those elements of pPJV7563 and/or pPJV1671 themselves may be employed. In another preferred embodiment the corresponding elements of the construct pPML7789 may be employed and the location of these is indicated in particular in FIG. 21 and the sequence listing provided herein. In further preferred instances, the corresponding elements of the vectors pPJV2012 and pPJV7788 may be employed and in particular those elements indicated in Tables 6 and 8 may be employed. Functional fragments and variants of the elements of the vectors pPML7789, pPJV2012 and pPJV7788 may also be employed in constructs of the invention.

In one preferred instance, a construct of the invention comprises:
(i) the sequence of the vector pPJV7563 provided as SEQ ID No:14;
(ii) a sequence with 60% sequence identity to the sequence of (i),
   apart from insertion of the sequence encoding the influenza antigen, an immunogenic fragment or immunogenic variant into the sequence of (i) or (ii) so that it is operably linked to the chimeric promoter. In a preferred instance, a coding sequence of such a construct encodes a HA antigen, an immunogenic variant thereof or an immunogenic fragment of either.

In further preferred instances of the invention a construct is provided where the vector comprises the sequence of the vector pPJV1671 provided as SEQ ID No: 54 or a sequence with 60% sequence identity thereto. IN another preferred instance, the vector comprises the sequence of the vector pPML7789 provided as SEQ ID No: 59 or a sequence with 60% sequence identity thereto.

In a preferred instance, the CMV promoter, untranslated leader sequence, rat insulin intron A, untranslated leader sequence, HBV enhancer and/or rabbit poly A sequence of pPJV7563 and/or pPJV1671 are employed or a functional variant or fragment of such sequences. In an especially preferred instance the rat insulin intron A intron and/or HBV enhancer of pPJV7563 and/or pPJV1671 are employed or a functional fragment or variant of such sequences. In particular, both the rat insulin intron A and HBV enhancer are employed or a functional fragment or variant of such sequences is employed. In another preferred instance the corresponding sequences of pPML7789, pPJV2012, pPJV7788, functional fragments of such sequences or variants of such sequences may be employed.

In instances where the construct encodes several polypeptides and in particular antigens, particular sequences may be omitted to reduce the size of the construct. For instance, the constructs may lack an enhancer and/or an untranslated leader sequence, particularly where three, four, five or more antigens are encoded. In other constructs encoding the same number of antigens these sequences may still be present.

In a particularly preferred instance, the vector pPJV7563 vector is used to clone the desired coding sequences into and in particular the coding sequences for an antigen such as, for instance, any of the antigens mentioned herein. In a preferred instance a desired influenza antigen, fragment or variant thereof is cloned into the vector. Modifications may in some cases be made to pPJV7563. For instance, the Kanamycin resistance gene may be swapped for an alternative gene and in particular an alternative selectable or screenable marker. Any of the nucleic acid constructs of the invention may comprise selectable markers. The pUC19 plasmid backbone of pPJV7563 may be modified or replaced with a different plasmid backbone. The specific elements of pPJV7563 may, for instance, be swapped with any elements fulfilling the same function and in particular functional variants or fragments of the sequences in pPJV7563. In some instances pPJV7563 may be modified by replacement of a particular element with any of the elements referred to herein which have the same function as the element to be replaced. Similar modifications may be to pPJV1671 when it is being employed. Similar modifications may also be made to any of the vectors mentioned herein and in particular pPML7789, pPJV2012 and pPJV7788. The coding sequences of pPML7789, pPJV2012 and pPJV7788 may be swapped for any of the coding sequences mentioned herein. In the case of pPJV2012 and pPJV7788 one or both coding sequences of the vector may be swapped.

In one preferred embodiment a nucleic acid construct of the invention comprises:
(i) the sequence of the vector pPJV7563 provided as SEQ ID No:14;
(ii) a sequence with 60% sequence identity to the sequence of (i),
   apart from insertion of the desired coding sequences so that they are operably linked to the chimeric promoter. In a preferred instance, the coding sequences encode an antigen. In particular, a coding sequence encoding an influenza antigen, an immunogenic fragment or immunogenic variant is inserted into the sequence of (i) or (ii) so that it is operably linked to the chimeric promoter.

The construct may have 60% sequence identity to the sequence of (i) taking into account the inserted coding sequences or disregarding them. The construct may have any of the values of sequence identity specified herein and in particular, at least 70%, preferably at least 80%, more preferably at least 90% and even more preferably at least 95%. In a preferred embodiment the construct is pPJV7563 apart from insertion of the coding sequences.

Additional sequences may be added to the sequence of pPJV7563 to generate further constructs of the invention. For instance, one or more chimeric promoters of the invention may be added together with any of the sequences mentioned herein which may be in operable linkage to such a promoter. The cloning site of pPJV7563 may be modified to allow the cloning of different restriction fragments and in particular the cloning of coding sequences as different fragments. Similar modifications may be made to any of the vectors mentioned herein and in particular to pPML7789, pPJV2012 and pPJV7788.

In another preferred instance, a construct of the invention may be made by replacing some or all of the coding sequences of pPJV1671 encoding an antigen with sequences encoding a different antigen. Again, any of the modifications discussed above in relation to pPJV7563 may also be made to modify pPJV1671. Such modifications may also be made to pPML7789. The coding sequences of pPJV2012 and pPJV7788 may also be replaced with other desired coding sequences and in particular those encoding an antigen.

In other preferred instances, the construct of the invention comprises the:

sequence of the vector pPJV1671 provided as SEQ ID No: 54 or a sequence with 60% sequence identity thereto;

sequence of the vector pPML7789 provided as SEQ ID No: 59 or a sequence with 60% sequence identity thereto;

sequence of the vector pPJV2012 provided by SEQ ID No: 61 or a sequence with 60% sequence identity thereto;

sequence of the vector pPJV7788 provided by SEQ ID No: 62 or a sequence with 60% sequence identity thereto.

In such instances, the construct may have any of the percentage sequence identities specified herein and in particular those specified above in relation to pPJV7563.

A construct of the invention may comprise any of the elements specified in the Tables herein and in particular those specified in Tables 3, 6 and 8. Functional fragments of such sequences and variants of such sequences and their fragments may also be employed. In a preferred instance, where the vector is an adjuvant vector one or more of the elements specified by Tables 6 and/or 8 may be present or a fragment or variant of such a sequences.

The invention also provides a method for generating a construct of the invention comprising inserting coding sequences for a polypeptide and in particular an antigen into a vector of the invention lacking such sequences. The coding sequences may encode any of the antigens mentioned herein. In a preferred instance, the invention provides such a method comprising inserting the chosen coding sequences into pPJV7563, pPJV1671 or one of the modified versions of the vectors discussed herein. They may be inserted into pPML7789, pPJV2012 and/or pPJV7788 and in particular pPML7789. In some cases an additional coding sequence may be inserted and/or a coding sequence already present may be replaced with a different coding sequence.

The invention also provides a promoter sequence and a coding sequence operably linked to the promoter, where the construct further comprises:
(a) a non-translated leader sequence which is derived from HBVpreS2 antigen sequence, HBV e-antigen sequence or HSV type 2gD antigen sequence, which is in operable linkage with the coding sequence and promoter which is heterologous to the coding sequence; and/or
(b) an enhancer sequence 3′ of and operably linked to the coding sequence, where the enhancer sequence is derived from a 3′ UTR of an HBsAg sequence or a 3′ UTR of a simian CMV immediate early gene sequence, and the coding sequence is heterologous to the 3′ enhancer sequence.
   The various elements of the vector may be any of those specified herein. In one instance, the promoter sequence (i) is selected from the hCMV immediate early promoter sequence, Pseudorabies virus promoter sequence and Rous sarcoma virus promoter sequence. In another, the promoteris one of the chimeric promoters discussed herein.

In another instance, the invention provides a purified, isolated chimeric promoter where the chimeric promoter is any of those defined herein.

A polynucleotide construct of the invention may be substantially free of or associated with cells or with cellular material. It may be in substantially isolated form, or it may be in substantially purified form, in which case it will generally comprise at least 90% e.g. at least 95%, 98% or 99% of the polynucleotide or dry mass in the preparation.

The present nucleic acid molecules may be delivered to suitable host cells, for expression of a polynucleotide in operable linkage with the promoter. Preferably the host cells are mammalian cells, in particular human cells. Suitable methods for delivery of nucleic acids to such cells are known in the art and include, for example, dextran mediated transfection, calcium phosphate precipitation, electroporation and direct microinjection into nucleii. Thus, the invention provides a cell transformal with a vector of the invention.

As described above, a nucleic acid coding sequence in a construct may encode a therapeutically relevant polypeptide. The present constructs may therefore be used for nucleic acid immunisation or gene therapy using standard gene delivery protocols. Suitable methods for gene delivery are known in the art, as discussed below. The nucleic acid molecules can be delivered either directly to a subject, or alternatively, delivered ex vivo to cells derived from the subject whereafter the cells are reimplanted in the subject. In a preferred instance, the constructs are delivered directly to the subject where the encoded polypeptide is an antigen, particularly an influenza antigen. Any of the delivery routes mentioned herein may be employed and in particular transdermal delivery. The adjuvant constructs of the invention may be administered to enhance an immune response against an antigen and in particular against an antigen expressed from a construct of the invention.

The invention also provides for the use of a nucleic acid construct of the invention or a population of nucleic acid constructs of the invention or coated particles of the invention in the manufacture of a medicament for nucleic acid immunisation. The medicament may be one which is to be delivered by injection, transdermal particle delivery, inhalation, topically, orally, intranasally or transmucosally. In a preferred instance, the medicament is to be delivered by needleless injection.

For use in nucleic acid immunisation or gene therapy, the nucleic acid constructs may be formulated as conventional pharmaceutical preparations. This can be done using standard pharmaceutical formulation chemistries and methodologies, which are available to those skilled in the art. For example, compositions containing one or more nucleic acid sequences (e.g., present in a suitable vector form such as a DNA plasmid) can be combined with one or more pharmaceutically acceptable excipients or vehicles to provide a liquid preparation. Thus also provided is a pharmaceutical composition comprising a nucleic acid construct of the invention and a pharmaceutically acceptable carrier or excipient. In a preferred instance a pharmaceutical composition may comprise a plurality of constructs of the invention or a population of constructs of the invention including any of those mentioned herein.

Auxiliary substances, such as wetting or emulsifying agents, pH buffering substances and the like, may be present in the excipient or vehicle. These excipients, vehicles and auxiliary substances are generally pharmaceutical agents which may be administered without undue toxicity and which, in the case of vaccine compositions will not induce an immune response in the individual receiving the composition. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. It is also preferred, although not required, that the preparation will contain a pharmaceutically acceptable excipient that serves as a stabilizer, particularly for peptide, protein or other like molecules if they are to be included in the composition. Examples of suitable carriers that also act as stabilizers for peptides include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium.phosphates, citric acid, tartaric acid, glycine, high molecular weight polyethylene glycols (PEGs), and combination thereof. A thorough discussion of pharmaceutically acceptable excipients, vehicles and auxiliary substances is available in REMINGTON'S PHARMACEUTICAL SCIENCES (Mack Pub. Co., N.J. 1991), incorporated herein by reference.

Certain facilitators of nucleic acid uptake and/or expression (“transfection facilitating agents”) can also be included in the compositions, for example, facilitators such as bupivacaine, cardiotoxin and sucrose, and transfection facilitating vehicles such as liposomal or lipid preparations that are routinely used to deliver nucleic acid molecules. Anionic and neutral liposomes are widely available and well known for delivering nucleic acid molecules (see, e.g., Liposomes: A Practical Approach, (1990) RPC New Ed., IRL Press). Cationic lipid preparations are also well known vehicles for use in delivery of nucleic acid molecules. Suitable lipid preparations include DOTMA (N-[1-(2,3-dioleyloxy)propyl]-N,N,N-trimethylammonium chloride), available under the tradename Lipofectin™, and DOTAP (1,2-bis(oleyloxy)-3-(trimethylammonio)propane), see, e.g., Feigner et al. (1987) Proc. Natl. Acad. Sci. USA 84:7413-7416; Malone et al. (1989) Proc. Natl. Acad. Sci. USA 86:6077-6081; U.S. Pat. Nos. 5,283,185 and 5,527,928, and International Publication Nos WO 90/11092, WO 91/15501 and WO 95/26356. These cationic lipids may preferably be used in association with a neutral lipid, for example DOPE (dioleyl phosphatidylethanolamine). Still further transfection-facilitating compositions that can be added to the above lipid or liposome preparations include spermine derivatives (see, e.g., International Publication No. WO 93/18759) and membrane-permeabilizing compounds such as GAL4, Gramicidine S and cationic bile salts (see, e.g., International Publication No. WO 93/19768).

Alternatively, the nucleic acid molecules of the present invention may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyomithine, spermine, spermidine, as well as conjugates of these molecules. In a preferred embodiment, constructs of the invention are precipitated onto carriers in the presence of a nucleic acid condensing agent and a metal ion chelating agent. Preferred condensing agents include cationic polymers, in particular polyamines, and in particular a polyargine or a polylysine. In a preferred instance the polyamine is (Arg)₄ or (Arg)₆. Reference may be made to the techniques discussed in WO2004/208560 which may be employed to generate coated carrier particles of the invention.

Once formulated the compositions can be delivered to a subject in vivo using a variety of known routes and techniques. For example, the liquid preparations can be provided as an injectable solution, suspension or emulsion and administered via parenteral, subcutaneous, intradermal, intramuscular, intravenous injection using a conventional needle and syringe, or using a liquid jet injection system. Liquid preparations can also be administered topically to skin or mucosal tissue, or provided as a finely divided spray suitable for respiratory or pulmonary administration. Other modes of administration include oral administration, suppositories, and active or passive transdermal delivery techniques.

Alternatively, the compositions can be administered ex vivo, for example delivery and reimplantation of transformed cells into a subject are known (e.g., dextran-mediated transfection, calcium phosphate precipitation, electroporation, and direct microinjection into nuclei).

The compositions are administered to a subject in an amount that is compatible with the dosage formulation and that will be prophylactically and/or therapeutically effective. An appropriate effective amount will fall in a relatively broad range but can be readily determined by one of skill in the art by routine trials. The “Physicians Desk Reference” and “Goodman and Gilman's The Pharmacological Basis of Therapeutics” are useful for the purpose of determining the amount needed. For example, it is generally expected that an effective dose of the polynucleotide will fall within a range of about 0.001 to 1000 μg, preferably from 0.001 to 100 μg, more preferably 0.01 to 10.0 μg. In some instances the dose may be from 0.1 to 100 μg, preferably from 0.5 to 25 μg. In cases where the dose is administered via needleless injection, the dose may in some cases be from 0.1 to 25 μg, preferably from 0.5 to 10 μg and more preferably from 1 to 5 μg. In particular, the dose may be 4 μg. In some cases, the dose may be given via a plurality of needleless injections such as, for instance, in one, two, three, four or five needleless injections.

In some cases after an initial administration a subsequent administration of the construct may be performed. In particular, following an initial immunization a subject may be given a booster immunization. The booster immunization may be, for instance, a dose chosen from any of those mentioned herein. The subject administration may, for instance, be at least a week, two weeks, a month, two months or six months after the initial administration.

In one instance, a nucleic acid construct of the invention may be used in conjunction with another nucleic acid construct. In one case, the nucleic acid construct may be one of those described herein for the expression of an adjuvant and the other construct may be a construct encoding one or more antigens. In a preferred case, both constructs may employ the chimeric promoters of the invention. Where two or more agents are given herein they may in particular be administered separately, simultaneously or sequentially.

In the case where one construct expresses an adjuvant and the other an antigen or antigens, the antigens may in particular be from HSV, HPV, or Hepatitis virus (particularly Hepatitis B virus). The antigens may in particular be the HSV ICP0, ICP4, ICP 22 and/ICP 27 antigens and preferably all four. In an especially preferred case, the antigens may be an influenza antigen including any of those mentioned herein and in particular HA, NA and/or M2 and in particular HA and NA and especially HA. In cases where such antigens are expressed, the adjuvant construct will in particular express LTA and/or LTB and in particular both. Any of the adjuvant constructs mentioned herein may be employed simultaneously, separately or sequentially with any of the constructs encoding an antigen.

Any two entities of the invention may be administered separately, sequentially or simultaneously. The two constructs may be administered separately, simultaneously or sequentially. The two may be administered in the same or different compositions. In particular, where one construct has an adjuvant effect the two will be delivered so that an adjuvant effect is seen, that is the immune response generated will be greater and/or for a longer period than if the adjuvant had not been administered with the antigen. In a preferred instance, the two constructs may be delivered in the same composition, preferably on the same carrier particles. In other cases carrier particles carrying a construct may be mixed with carrier particles carrying another construct. Any such administration schemes may be employed for any of the combinations of a plurality of construct discussed herein.

In a preferred embodiment, the nucleic acid constructs of the invention are delivered to target cells using a particle-mediated delivery technique. Particle mediated methods for delivering nucleic acid preparations are known in the art.

Thus, in a preferred instance the invention provides coated particles which comprise carrier particles coated with a nucleic acid construct of the invention or a population of nucleic acid constructs of the invention. In particular, the coated particles are suitable for delivery from a particle-mediated delivery device.

Particles for particle mediated delivery may be formed by coating the present nucleic acid molecules onto carrier particles (e.g., core carriers) using a variety of techniques known in the art. Carrier particles are selected from materials which have a suitable density in the range of particle sizes typically used for intracellular delivery from a particle-mediated delivery device. Typically carrier particles have a diameter of from 0.1 to 5 μm, for example 0.5 to 3 μm, preferably 1 to 2 μm. In some cases, the particles may have a diameter of from 1 to 3 μm. The optimum carrier particle size will, of course, depend on the diameter of the target cells.

Usually carrier particles are selected from inert metals. The metals are inert in that they are not physiologically active. For the purposes of the invention Iron, Cobalt, Nickel, Copper, Silver, Cadmium, Hafnium, Tantalum, Tungsten, Platinum, Gold, and Stainless Steel may, for instance may be used and in particular tungsten, gold, platinum and iridium carrier particles can be used. Tungsten and gold particles are preferred. Thus, in a preferred instance, the invention provides coated carrier particles which are gold or tungsten. Tungsten particles are readily available in average sizes of 0.5 to 2.0 μm in diameter. Although such particles have optimal density for use in particle acceleration delivery methods, and allow highly efficient coating with DNA, tungsten may potentially be toxic to certain cell types. Gold particles or microcrystalline gold (e.g., gold powder A1570, available from Engelhard Corp., East Newark, N.J.) will also find use with the present methods. Gold particles provide uniformity in size (available from Alpha Chemicals in particle sizes of 1-3 μm, or available from Degussa, South Plainfield, N.J. in a range of particle sizes including 0.95 μm) and reduced toxicity. Microcrystalline gold provides a diverse particle size distribution, typically in the range of 0.1-5 μm. However, the irregular surface area of microcrystalline gold provides for highly efficient coating with nucleic acids.

A number of methods are known and have been described for coating or precipitating DNA or RNA onto gold or tungsten particles. Most such methods generally combine a predetermined amount of gold or tungsten with plasmid DNA, CaCl₂ and spermidine. The resulting solution is vortexed continually during the coating procedure to ensure uniformity of the reaction mixture. After precipitation of the nucleic acid, the coated particles can be transferred to suitable membranes and allowed to dry prior to use, coated onto surfaces of a sample module or cassette, or loaded into a delivery cassette for use in particular particle-mediated delivery instruments.

As an alternative, the polynucleotides of the invention can be formulated as a particulate composition. Formulation can be carried out using the above-described standard pharmaceutical formulation chemistries. For example, the polynucleotides can be combined with one or more pharmaceutically acceptable excipients or vehicles to provide a suitable composition. The formulated compositions are then prepared as particles using standard techniques such as by simple evaporation (air drying), vacuum drying, spray drying, freeze drying (lyophilisation), spray-freeze drying, spray coating, precipitation, supercritical fluid particle formulation, and the like. If desired, the resultant particles can be densified using the techniques described in commonly owned International Publication No. WO 97/48485, incorporated herein by reference.

These methods can be used to obtain nucleic acid particles having a size ranging from about 0.01 to about 250 μm, preferably about 10 to about 150 μm, and most preferably about 20 to about 60 μm; and a particle density ranging from about 0.1 to about 25 g/cm³, and a bulk density of about 0.5 to about 3.0 g/cm³, or greater.

Once formed, the particles comprising the nucleic acid molecules may be packaged in single unit dosages or multidose containers. The invention therefore also provides a dosage receptacle for a particle mediated delivery device comprising coated particles of the invention. Such containers may comprise an hermetically sealed container enclosing a suitable amount of the particles. The particles can be packaged as a sterile formulation, and the hermetically sealed container can thus be designed to preserve the sterility of the formulation until use in delivery to a subject. The containers are preferably adapted for direct use in a particle mediated delivery device. Typically such containers take the form of capsules, foil pouches, sachets, cassettes and the like. The particle delivery devices can also be provided in a preloaded condition containing a suitable dosage of the particles. The preloaded device may then also be prepackaged in a hermetically sealed container.

The container in which the particles are packaged can further be labelled to identify the composition and provide relevant dosage information. In addition, the container can be labelled with a notice in the form prescribed by a governmental agency, for example, the Food and Drug Administration, wherein the notice indicates approval by the agency under Federal Law of the manufacture, use or sale of the nucleic acid preparation contained therein for human administration.

The invention also provides a particle mediated delivery device loaded with coated particles of the invention. Preferably, the delivery device is a needleless syringe. Particle acceleration devices, suitable for particle-mediated delivery are known in the art. For example, current gene gun devices employ an explosive, electric or gaseous discharge to propel coated carrier particles towards target cells. The coated carrier particles can be releasably attached to a movable carrier sheet, or removably attached to a surface along which a gas stream passes, lifting the particles from the surface and accelerating them toward the target. An example of a gaseous discharge device is described in U.S. Pat. No. 5,204,253. An explosive-type device is described in U.S. Pat. No. 4,945,050. One example of an electric discharge apparatus suitable for use herein is described in U.S. Pat. No. 5,120,657. Another electric discharge apparatus is described in U.S. Pat. No. 5,149,655. The disclosure of all of these patents is incorporated herein by reference in their entireties.

Particles may also be administered using a needleless syringe device, such as those described in U.S. Pat. No. 5,630,796 to Bellhouse et al (“the PowderJect® needleless syringe device”) and in International Publication Nos. WO 94/24263, WO 96/04947, WO 96/12513 and WO 96/20022, all of which are incorporated herein by reference.

Devices such as the one described in U.S. Pat. No. 5,630,796 may be provided as a pen-shaped instrument containing, in linear order moving from top to bottom, a gas cylinder, a particle cassette or package, and a supersonic nozzle with an associated silencer medium. The particles are provided within a suitable container, e.g. a cassette formed by two rupturable polymer membranes that are heat-sealed to a washer-shaped spacer to form a self-contained sealed unit. Membrane materials can be selected to achieve a specific mode of opening and burst pressure that dictate the conditions at which the supersonic flow is initiated.

In operation, the device is actuated to release the compressed gas from the cylinder into an expansion chamber within the device. The released gas contacts the particle cassette and, when sufficient pressure is built up, suddenly breaches the cassette membranes sweeping the particles into the supersonic nozzle for subsequent delivery. The nozzle is designed to achieve a specific gas velocity and flow pattern to deliver a quantity of particles to a target surface of predefined area. The silencer is used to attenuate the noise produced by the supersonic gas flow.

The delivery system described in International Publication No. WO 96/20022 also uses the energy of a compressed gas source to accelerate and deliver powdered compositions. However, it is distinguished from the system of U.S. Pat. No. 5,630,796 in its use of a shock wave instead of gas flow to accelerate the particles. More particularly, an instantaneous pressure rise provided by a shock wave generated behind a flexible dome strikes the back of the dome, causing a sudden eversion of the flexible dome in the direction of a target surface. This sudden eversion catapults a powdered composition (which is located on the outside of the dome) at a sufficient velocity, thus momentum, to penetrate target tissue, e.g., oral mucosal tissue. The powdered composition is released at the point of full dome eversion. The dome also serves to completely contain the high-pressure gas flow which therefore does not come into contact with the tissue. Because the gas is not released during this delivery operation, the system is inherently quiet. This design can be used in other enclosed or other wise sensitive applications for example, to deliver particles to minimally invasive surgical sites.

Particles may be delivered in vivo directly to a subject, or ex vivo to cells taken from a subject, the transformed cells then being reimplanted in the subject. For in vivo delivery, particle injection is typically subcutaneously, epidermally, intradermally, intramucosally (e.g. nasally, rectally and/or vaginally), intraperitoneally, intravenously, orally or intramuscularly. Preferably, delivery is to terminally differentiated cells; however, the particles can also be delivered to non-differentiated, or partially differentiated cells such as stem cells of blood and skin fibroblasts. Most preferably, delivery is to skin epidermal cells.

The particles are administered to a subject in a manner compatible with the dosage formulation and in an amount that will be prophylactically and/or therapeutically effective. A “therapeutically effective amount” of the present particulate compositions will be sufficient to bring about treatment or prevention of disease or condition symptoms, and will fall in a relatively broad range that can be determined by routine trials. Generally the particles are delivered in an amount of from 0.001 to 1000 μg, more preferably 0.01 to 10.0 μg of nucleic acid per dose. However, the exact amount necessary will vary depending on the age and general condition of the individual being treated and the particular nucleotide sequence selected, as well as other factors. An appropriate effective amount can be readily determined through clinical testing. The “Physicians Desk Reference” and “Goodman and Gilman's The Pharmacological Basis of Therapeutics” are useful for the purpose of determining the amount needed.

### Assays

### Comparative Expression Assay

A suitable test for element utility determines the effect the element has on expression of a polypeptide. In a preferred instance the polypeptide may be an influenza antigen, variant thereof or fragment of either. The basis of comparison for testing utility of the elements is a ‘base vector’, generally (unless otherwise noted) a plasmid with a hCMV promoter, hCMV exon 1, 9 bases of hCMV exon 2, the 5′ UTR from HBV preS2 and the rabbit beta-globin polyadenylation region, positioned to drive expression of a coding sequence. Typically, the base vector is pPJV7384, pPJV 7401, pPJV 7450 or pPJV7533. In some instances, any of the vectors mentioned herein with the above mentioned elements may be employed as a base vector. In one instance, pPJV1671 may be employed as a base vector. pPJV2012, pPJV7788 and pPML7789 may also be employed as base vectors.

The basic vector, heterologous introns and 3′ UTRs are added, or promoter sequences, exons, 5′ UTRs and polyA sites are swapped into the base vectors to create test expression vectors. Any of the elements of the vectors discussed herein may be swapped with a test sequence and compared with a base sequence. Thus, functional variants or fragments can be tested.

The base vectors and test vectors are transformed into suitable host cells and the cells analysed for polypeptide expression levels. Preferably mammalian host cells are used. Suitable cells include mammalian HEK 293T, CHO, HeLa, BHK, 3T3 or COS cells. In some instances, SSC15 or B16 cells may be employed.

Typically, a functional element causes expression which is comparable to the base vector, for example at least the same as or greater. Preferably expression is tested in more than one cell type and with more than one coding sequence. In some instances, a functional element may cause expression which is slightly less than the base vector such as, for instance, at least 25%, in particular at least 50%, preferably at least 60%, more preferably at least 70%, still more preferably at least 80%, even more preferably at least 90% and still more preferably at least 90% of the expression of the base vector. Typically, a variant or fragment of a particular element may still be considered to represent a functional variant or fragment of a particular element if it shows such levels of expression. However, preferably functional variants and fragments will give rise to higher expression than the base vector as discussed above. The percentage increase may, for instance, be any of the percentages mentioned above.

Suitable experimental protocols are provided, for example, in Examples 1 to 18 below.

### Comparative Immunogenicity Assay

Where the polypeptide to be expressed is an antigen, a further test may be carried out to identify functional or particularly preferred construct elements. In particular, such a test may be carried out where the antigen is, for instance, an influenza antigen, fragment thereof or variant of either. In the assay, the effect of an element on immune response is determined after delivery of an expression vector to a test organism. Antibody levels against the antigen are the easiest way to judge immune response. Groups of mice are vaccinated with the base vectors or test vectors constructed as above. Sera is collected after an appropriate amount of time and analyzed for antibody levels.

This experiment is performed twice, and the antibody levels from all the groups in both experiments are plotted. Functional elements will typically give rise to at least as high as or higher antibody titres in both experiments for a particular antigen than the base vector. Preferably, the result will be seen with more than one antigen to demonstrate the breadth of utility of the element(s) in that expression panel. In some instances, a functional element may give rise to an antibody titre which is which is slightly less than that seen with the base vector such as, for instance, at least 25%, in particular at least 50%, preferably at least 60%, more preferably at least 70%, still more preferably at least 80%, even more preferably at least 90% and still more preferably at least 90% of the antibody titre seen with the base vector. Typically, a variant or fragment of a particular element may still be considered to represent a functional variant or fragment of a particular element if it give rises to such an antibody titre. However, preferably functional variants and fragments will give rise to high titres than the base vector as discussed above. The percentage increase may, for instance, be any of the percentages mentioned above.

Adjuvant vectors may also be assessed by comparison of a test adjuvant vector with a standard adjuvant with both being administered with the same antigen. A comparison of the adjuvant effect of both vectors is made using the antigen administered alone as a control. Any of the adjuvant vectors mentioned herein may be employed as a standard. The percentage increase or decrease in adjuvant effect may be any of those levels mentioned herein.

Suitable experimental protocols are provided for example, in Example 14 below. Suitable protocols are also described in Examples 15 to 18 below.

### C. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Methods

### Standard PCR Conditions

The standard PCR conditions used for the construction of vectors were as follows: 1×PCR core buffer with 1.5 mM MgCl₂ (Promega Corporation, Madison, Wis.), 0.400 μM each of each primer, 200 μM of each dNTP (USB, Inc, Cleveland, Ohio), 2.5μ Taq polymerase (Promega Corporation, Madison, Wis.), 1.0 ng template DNA, water to 100 μl, and a mineral oil (Aldrich Chemical, Inc, Milwaukee, Wis.) overlay. The PTC-200 thermocycler (MJ Research, Inc, Waltham, Mass.) was programmed to run the following routine: 4′@95° C., 30 cycles of (1′@95° C./1′15″@55° C./1′@72° C.), 10′@72° C., 4° C. hold). The amplification products were removed from the PCR reaction by using the QIAquickâPCR Purification Kit (Qiagen Inc, Valencia, Calif.) prior to cutting with restriction enzymes (New England Biolabs, Beverly, Mass.).

All PCR products were sequenced after cloning to ensure fidelity of the amplification.

### Example 1

### Construction of Hepatitis B Virus Surface Antigen (HBsAg) Vector Panels

A number of plasmid expression vectors were constructed for expression of HBsAg.

### Starting Materials

(i) pWRG7128 (Roy, M, et. al. Vaccine (2001) 19: 764-778), which contains the hCMV immediate early promoter sequence, the first exon, first intron, and a partial second exon of the hCMV major immediate early gene, the HBsAg coding sequence with flanking regions (HBV preS2 5′UTR derived sequence and 3′ posttranscriptional response element) and the bovine growth hormone polyadenylation region (BGHpA)

(ii) pPJV7284, a derivative of pWRG7128 that exchanges the rabbit globin polyadenylation region (RBGpA) for BGHpA.

(a) pPJV7384 (CMV(No Intron), HBV preS2 5′ UTR and RBGpA)

pWRG7128 was PCR amplified with JF93 (SEQ ID NO:15) and F110 (SEQ ID NO:16) using standard conditions and cut with Sal1 and BamH1 to isolate an insert fragment containing the CMV promoter, exon 1 and part of the exon 2 sequence. pAM6 (ATCC, Mannassas, Va.) was cut with BamH1 and BstX1 to isolate an insert fragment that contained the 5′-UTR of HBsAg, and roughly 70% of the HBsAg coding region. pJV7284 was cut with Sal1 and BstX1 to generate a vector fragment into which the two insert fragments were ligated, resulting in pJV7293.

pWRG7128 was PCR amplified with primers GW1 (SEQ ID NO:17) and JF254 (SEQ ID NO:18) and cut with BstX1 and Bgl2 to isolate an insert fragment that contained the 3′-end of the HBsAg coding region. pPJV7293 was cut with BstX1 and Bgl2 to generate a vector fragment into which the insert fragment was ligated, resulting in vector pPJV7384.

(b) pPJV7382 (CMV(No Intron), HBsAg 3′ UTR, HBV preS2 5′ UTR and RBGpA)

pPJV7293 was cut with Xho1 and Xba1 to generate an insert fragment containing the CMV promoter/exons and the 5′-UTR with 5′-end of the HBsAg coding sequence. pWRG7128 was cut with Xba1 and Bcl1 to generate an insert fragment containing the majority of the HBsAg coding sequence and the 3′-UTR. pPJV7284 was cut with Xho1 and Bgl2 to generate a vector fragment into which the two insert fragments were ligated, resulting in pPJV7382.

(c) pPJV7389 (CMV (RIA), HBsAg 3′ UTR, HBV preS2 5′ UTR and RBGpA)

The rat insulin intron A (RIA) was PCR amplified out of plasmid p5′rIns with primers GW150 (SEQ ID NO:19) and JF255 (SEQ ID NO:20). The PCR product was cut with BamH1 and inserted into BamH1 linearized pPJV7382, resulting in pPJV7389.

(d) pPJV7387 (CMV(RIA), HBV preS2 5′UTR and RBGpA)

pPJV7384 was cut with BstX1 and EcoR1 to generate an insert fragment containing the 3′ end of the HBsAg coding region and RBGpA. pPJV7389 was cut with BstX1 and EcoR1 to generate a vector fragment into which the insert fragment was ligated, resulting in pPJV7387.

### Example 2

### Construction of Herpes Simplex Virus Glycoprotein D Antigen (HSVgD) Vector Panels

A number of plasmid expression vectors were constructed for expression of HSVgD.

### Starting Materials

(a) pPJV7334, a derivative of pWRG7284 (pPJV 7284) that replaces the HBsAg coding sequence with an in-frame Nhe1 directly downstream of the ATG start codon, followed by a stuffer fragment with a BamH1 immediately 5′ of the HBV Enh (Enhancer).

(b) pWRG7202, a derivative of pGem3Z (Promega) with a stuffer fragment that allows the fusion of a coding sequence to the human tissue plasminogen activator (TPA) signal peptide downstream of a Nhe1 site.

(a) pPJV7392(CMV(native intron), HBsAg 3′ UTR, HBsAg 5′UTR and RBGpA)

The coding region for HSV2 gD was PCR amplified out of a viral DNA stock (Advanced Biotech, Inc, Columbia, Md.) using primers DS1 (SEQ ID NO:21) and DA1 (SEQ ID NO:22) and was cut with Nhe1 and EcoR1 to generate an insert fragment. pWRG7202 was cut with Nhe1 and EcoR1 to generate a vector fragment into which the insert fragment was ligated, resulting in pPJV7391.

pPJV7391 was cut with Nhe1 and Bgl2 to generate an insert fragment containing the HSV2 gD coding sequence. pPJV7334 was cut with Nhe1 and BamH1 to generate a vector fragment into which the insert fragment was ligated, resulting in pPJV7392. This vector consists of the following expression elements: the hCMV immediate early promoter sequence, the first exon, first intron, and a partial second exon of the hCMV major immediate early gene, the 5′-UTR from HBsAg, the coding sequence for HSV2 gD gene, the 3′-UTR from HBsAg, and RBGpA.

(b) pPJV7399 (CMV(no intron), HBsAg 3′ UTR HBsAg 5′UTR and RBGpA)

An intronless version of pPJV7392 was constructed as follows. pPJV7384 was cut with HindIII and Nde1 to isolate an insert fragment containing the 5′ ends of the kanamycin resistance gene and the CMV promoter. pPJV7384 was cut with Nde1 and Ssp1 to isolate an insert fragment containing the 3′ end of the CMV promoter, the CMV exon1/2 and 5′ end of the 5′-UTR from HBsAg. These insert fragments were inserted into pPJV7392 from which the Hind3-Ssp1 fragment was removed, resulting in pPJV7399.

(c) pPJV7400 (CMV(RIA), HBsAg 3′ UTR, HBsAg 5′UTR and RBGpA)

A RIA version of pPJV7392 was constructed as follows. pPJV7384 was cut with HindIII and Nde1 to isolate an insert fragment containing the 5′ ends of the kanamycin resistance gene and CMV promoter. pPJV7387 was cut with Nde1 and Ssp1 to isolate an insert fragment containing the 3′ end of the CMV promoter, the CMV exon1/2(partial), RIA, and 5′ end of the 5′-UTR from HBsAg. These insert fragments were inserted into pPJV7392 from which the Hind3-Ssp1 fragment was removed, resulting in pPJV7400.

(d) pPJV7401 (CMV(no intron), HBsAg 5′UTR and RBGpA)

A 3′-UTR-less version of pPJV7399 was constructed as follows. pPJV7391 was cut with Bsp120I and Bgl2 to isolate an insert fragment containing the 3′ end of the HSV2 gD gene. pPJV7284 was cut with Bgl2 and EcoR1 to isolate the RBGpA signal. These insert fragments were inserted into pPJV7399 from which the Bsp1201-EcoR1 fragment was removed, resulting in pPJV7401.

(e) pPJV7402 (CMV(RIA), HBsAg 5′UTR and RBGpA)

A 3′-UTR-less version of pPJV7400 was constructed as follows. pPJV7391 was cut with Bsp120I and Bgl2 to isolate an insert fragment containing the 3′ end of the HSV2 gD gene. pPJV7284 was cut with Bgl2 and EcoR1 to isolate the RBGpA signal. These insert fragments were ligated into pPJV7400 from which the Bsp120I-EcoR1 fragment was removed, resulting in pPJV7402.

### Example 3

### Construction of Flu M2 Antigen Vector Panels

(a) pPJV7450 (CMV(No Intron), HBsAg 5′UTR and RBGpA)

A coding region for Flu M2 was PCR amplified out of plasmid pFL-M2 (Joel Haynes, pPJV) using primers JF301 (SEQ ID NO:23) and JF302 (SEQ ID NO:24) and was cut with Nhe1 and Bgl2 to generate an insert fragment. pPJV7401 was cut with Nhe1 and Bgl2 to generate a vector fragment into which the insert fragment was ligated, resulting in pPJV7450.

(b) pPJV7452 (CMV(no intron), HBsAg 3′ UTR, HBsAg 5′UTR and RBGpA)

A 3′ UTR fragment was PCR amplified out of pPJV7389 with primers JF84 (SEQ ID NO:25) and JF225 (SEQ ID NO:26) was cut with Bsp120I, filled with T4 DNA polymerase, and linkered with Bgl2 linkers (cat# 1036, New England Biolabs). The fragment was then cut with Bgl2 and EcoR1 to isolate an insert fragment containing the 3′ UTR of HBsAg and the RBGpA region. pPJV7450 was cut with Bgl2 and EcoR1 to generate a vector fragment into which the insert fragment was ligated, resulting in pPJV7452

(c) pPJV7458 (CMV(RIA) HBsAg 5′ UTR and RBGpA)

A version of pPJV7450 containing the RIA was constructed as follows: pPJV7389 was cut with BamH1 to isolate a RIA containing insert fragment. pPJV7450 was cut with BamH1 to generate a vector fragment into which the insert fragment was ligated, resulting in pPJV7458.

(d) pPJV7468 (CMV(RIA), HBsAg 3′ UTR, HBsAg 5′UTR and RBGpA)

A version of pPJV7458 containing the 3′ UTR of HBsAg was constructed as follows: pPJV7452 was cut with Bgl2 and EcoR1 to produce an insert fragment containing the HBsAg 3′ UTR and RBGpA. pPJV7458 was cut with Bgl2 and EcoR1 to generate a vector fragment into which the insert fragment was ligated, resulting in pPJV7468.

### Example 4

### Construction of Beta-Gal Vector Panels

(a) pPJV7488 (CMV(No Intron), HBsAg 3′ UTR, HBsAg 5′UTR and RBGpA)

CMV-beta (Clontech) was PCR amplified with primers JF335 (SEQ ID NO:27) and JF336 (SEQ ID NO:28) and cut with Nhe1 and Bgl2 to isolate an insert fragment coding for beta-galactosidase. pPJV7452 was cut with Nhe1 and Bgl2 to generate a vector fragment into which the insert fragment was ligated, resulting in pPJV7488.

(b) pPJV7533 (CMV(No Intron), HBsAg 5′UTR and RBGpA)

pPJV7450 was cut with Bgl2 and EcoR1 to isolate an insert fragment containing the RBGpA. pPJV7488 was cut with Bgl2 and EcoR1 to generate a vector fragment into which the insert fragment was ligated, resulting in pPJV7533.

(c) pPJV7551(CMV(RIA/NheI), HBsAg 3′ UTR, HBsAg 5′UTR and RBGpA)

pPJV7530 (see Example 5) was cut with Xho1 and BamH1 to isolate an insert fragment containing the CMV promoter through RIA. pPJV7488 was cut with Xho1 and BamH1 to generate a vector fragment into which the insert fragment was ligated, resulting in pPJV7551.

(d) pPJV7552(CMV(RIA/NheI), HBsAg 5′UTR and RBGpA)

pPJV7530 was cut with Xho1 and BamH1 to isolate an insert fragment containing the CMV promoter through to the RIA. pPJV7533 was cut with Xho1 and BamH1 to generate a vector fragment into which the insert fragment was ligated, resulting in pPJV7552.

### Example 5

### Construction of pPJV Expression (pPJV7563)

(a) pPJV7496

pPJV7389 was PCR amplified with primers JF357 (SEQ ID NO:29) and JF365 (SEQ ID NO: 30), treated with T4 DNA polymerase to blunt the ends, and cut with Sal1 to isolate an insert fragment coding for kanamycin resistance. pPJV7389 was cut with Ava1, treated with T4 DNA polymerase to blunt the ends, and cut with Sal1 to isolate a vector fragment into which the insert fragment was ligated, resulting in pPJV7496.

(b) pPJV7530

pPJV7389 was PCR amplified with primers 3F393 (SEQ ID NO:31) and JF406 (SEQ ID NO:32) and cut with Bgl2 and BamH1 to isolate an insert fragment containing the RIA devoid of an internal Nhe1 site. pPJV7496 was cut with BamH1 to prepare a vector fragment into which the insert fragment was ligated, resulting in pPJV7530.

(c) pPJV7549

pPJV7468 was cut with BamH1 and EcoR5 to isolate an insert fragment containing M2 and part of the HBV 3′ ENH. pPJV7530 was cut with BamH1 and EcoR5 to prepare a vector fragment into which the insert fragment was ligated, resulting in pPJV7549.

(d) pPJV7563

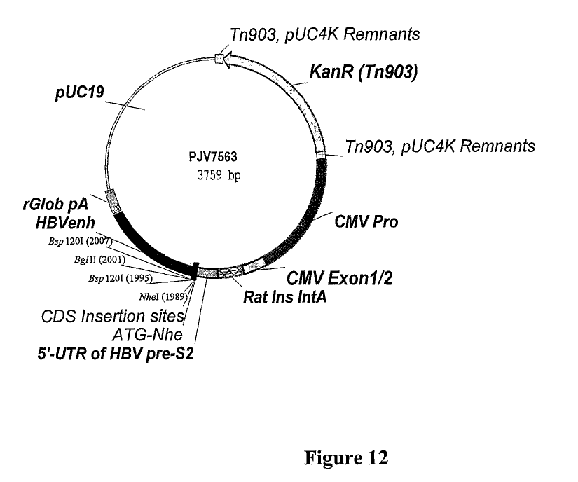

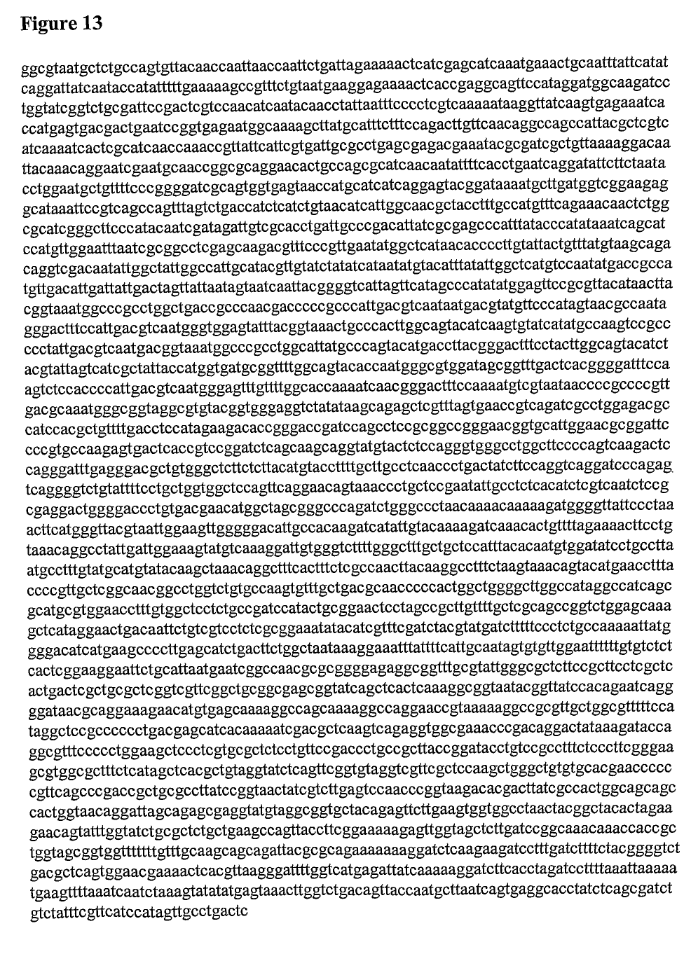

Primers JF256 (SEQ ID NO:33) and JF257 (SEQ ID NO:34) were annealed to prepare an insert fragment consisting of a multiple cloning site. pPJV7549 was cut Nhe1 and Bgl2 to prepare a vector fragment into which the insert fragment was ligated, resulting in pPJV7563. A pPJV7563 plasmid map is provided in FIG. 12. The base composition for the pPJV7563 plasmid is provided in FIG. 13. The components and their position in the plasmid pPJV7563 are as follows:

1-44 Transposon 903 sequences
45-860 Kanamycin resistance coding sequence from Transposon 903
861-896 Transposon 903 sequences
897-902 Sal1 site
903-1587 CMV promoter
1588-1718 untranslated leader sequence from the immediate-early gene of CMV
1719-1724 Fusion of BamH1 and BglII restriction enzymes
1725-1857 Rat insulin intron A
1858-1863 BamH1 site
1864-1984 HBV surface antigen 5′-untranslated leader
1985-1993 Synthetic start codon/Nhe1 cloning site
1994-2011 Synthetic cloning sites
2012-2544 HBV enhancer
2545-2555 Old vector sequence. No hits against NCBI databases
2556-2686 Rabbit beta-globin polyadenylation region
2687-3759 pUC19 vector sequence

### Example 6

### Construction of Signal Peptide Expression Panels using Human Secreted Alkaline Phosphatse (SEAP) and Human IgG Fc Fragment (hFc) as Model Antigens

(i) pPJV7507 (hTPAsp and SEAP)

pSEAP-Basic (Clontech) was PCR amplified with primers JF320 (SEQ ID NO:35) and JF321 (SEQ ID NO:36) then cut with Nhe1 and Bgl2 to isolate an insert fragment consisting of the human SEAP fragment. pPJV7079 (Macklin, et. al.) was cut with Nhe1 and Bgl2 to prepare a vector fragment into which the insert fragment was ligated, resulting in pPJV7507.

(ii) pPJV7508 (hTPAsp and hFc)

Human DNA was PCR amplified with primers JF386 (SEQ ID NO:37) and FcAS (SEQ ID NO:38) then cut with Nhe1 and Bgl2 to isolate an insert fragment consisting of the human IgG Fc fragment. pPJV7079 was cut with Nhe1 and Bgl2 to prepare a vector fragment into which the insert fragment was ligated, resulting in pPJV7508.

(iii) Preparation of Aprotinin Signal Peptide Coding Sequence

Synthetic oligo JF354 (SEQ ID NO:39) was PCR amplified with primers JF355 (SEQ ID NO:40) and JF356 (SEQ ID NO:41) to generate the coding sequence for the aprotinin signal peptide.

(iv) Preparation of Tobacco Extensin Signal Peptide Coding Sequence

Synthetic oligo JF348 (SEQ ID NO:42) was PCR amplified with primers JF349 (SEQ ID NO:43) and JF350 (SEQ ID NO:44) to generate the coding sequence for the tobacco extensin signal peptide.

(v) Preparation of Chicken Lysozyme Signal Peptide Coding Sequence

Synthetic oligo JF351 (SEQ ID NO:45) was PCR amplified with primers JF352 (SEQ ID NO:46) and JF353 (SEQ ID NO:47) to generate the coding sequence for the chicken lysozyme signal peptide.

(a) Flu M2 Antigen Signal Peptide Panels
pPJV7499 (CMV(no intron), HbsAg 5′ UTR, RBGpA. aprotinin s.p.)
pPJV7497 (CMV(no intron), HbsAg 5′ UTR, RBGpA, tobacco extensin s.p.)
PPJV7500 (CMV(no intron), HbsAg 5′ UTR, RBGpA, chicken lysosyme s.p.)

Coding sequences for the signal peptides were cut with Spe1 and Nhe1 to isolate insert fragments. pPJV7450 was cut with Nhe1 to prepare a vector fragment into which the insert fragments were ligated, resulting in pPJV7499 (aprotinin), pPJV7497 (tobacco extensin), and pPJV7500 (chicken lysozyme).

(b) SEAP Signal Peptide Panels

pPJV7513 (CMV(no intron), HbsAg 5′ UTR, RBGpA, aprotinin s.p.)

pPJV7512 (CMV(no intron), HbsAg 5′ UTR, RBGpA, tobacco extensin sp.)

pPJV7510 (CMV(no intron), HbsAg 5′ UTR, RBGpA, chicken lysosyme s.p.)

pPJV7499, 7497, and 7500 were cut with Xho1 and Nhe1 to isolate an insert fragment consisting of the CMV promoter through the signal peptide coding sequence of the plasmids. pPJV7507 was cut with Xho1 and Nhe1 to prepare a vector fragment into which the insert fragments were ligated, resulting in pPJV7513 (aprotinin), pPJV7512 (tobacco extensin), and pPJV7510 (chicken lysozyme).

(c) hFc Signal Peptide Panels

pPJV7524 (CMV(no intron), HbsAg 5′ UTR, RBGpA, aprotinin s.p.)

pPJV7525 (CMV(no intron), HbsAg 5′ UTR, RBGpA, tobacco extensin s.p.)

pPJV7526 (CMV(no intron), HbsAg 5′ UTR, RBGpA, chicken lysosyme signal peptide)

pPJV7499, 7497, and 7500 were cut with Xho1 and Nhe1 to isolate an insert fragment consisting of the CMV promoter through the signal peptide coding sequence of the plasmids. pPJV7508 was cut with Xho1 and Nhe1 to prepare a vector fragment into which the insert fragments were ligated, resulting in pPJV7524 (aprotinin), pPJV7525 (tobacco extensin), and pPJV7526 (chicken lysozyme).

### Example 7

### Construction of Human Secreted Alkaline Phosphatase (SEAP) Panels

(a) pPJV7531 (CMV(No Intron), HbsAg 5′ UTR, RBGpA, Chicken Lysosyme s.p.)

pPJV7510 was cut with Sal1 and Bgl2 to isolate an insert fragment containing the CMV promoter through lysozyme signal peptide. pPJV7450 was cut with Sal1 and Bgl2 to generate a vector fragment into which the insert fragment was ligated, resulting in pPJV7531.

(b) pPJV7554 (CMV(RIA/NheI), HbsAg 5′ UTR, RBGpA, chicken lysosyme s.p.)

pPJV7530 was cut with Xho1 and BamH1 to isolate an insert fragment containing the CMV promoter through RIA. pPJV7531 was cut with Xho1 and BamH1 to generate a vector fragment into which the insert fragment was ligated, resulting in pPJV7554.

(c) pPJV7568 (CMV(no intron), HBsAg 3′ UTR, HbsAg 5′ UTR, RBGpA, chicken lysosyme s.p.)

pPJV7563 was cut with Bgl2 and EcoR1 to isolate an insert fragment containing the HBV 3′-UTR and RBGpA. pPJV7531 was cut with Bgl2 and EcoR1 to generate a vector fragment into which the insert fragment was ligated, resulting in pPJV7568.

(d) pPJV7572 (CMV(RIA/NheI), HBsAg 3′ UTR, HbsAg 5′ UTR, RBGpA, chicken lysosyme s.p.)

pPJV7563 was cut with Bgl2 and EcoR1 to isolate an insert fragment containing the HBV 3′ UTR and RBGpA. pPJV7554 was cut with Bgl2 and EcoR1 to generate a vector fragment into which the insert fragment was ligated, resulting in pPJV7572.

### Example 8

### Construction of Beta-gal and HBsAg Vectors Using the Chicken Keratin and Chicken Cardiac Actin Introns

(a) pPJV7557 (Beta-gal, CMV(cA intron), HbsAg 3′ UTR, HbsAg 5′ UTR and RBGpA)

Chicken DNA was PCR amplified with primers JF430 (SEQ ID NO:48) and JF442 (SEQ ID NO:49) and cut with Bgl2 and BamH1 to isolate an insert fragment consisting of the intron and flanking exon sequences from chicken cardiac actin. pPJV7488 was cut with BamH1 to prepare a vector fragment into which the insert fragment was ligated, resulting in pPJV7557.

(b) pPJV7558 (Beta-gal, CMV(cK intron), HbsAg 3′ UTR, HbsAg 5′ UTR and RBGpA)

Chicken DNA was PCR amplified with primers JF421 (SEQ ID NO:50) and JF444 (SEQ ID NO:51) and cut with Bgl2 and BamH1 to isolate an insert fragment consisting of the intron and flanking exon sequences from the chicken keratin gene. pPJV7488 was cut with BamH1 to prepare a vector fragment into which the insert fragment was ligated, resulting in pPJV7558.

(c) pPJV7578 (HBsAg, CMV(cA intron), HbsAg 3′ UTR, HbsAg 5′ UTR and RBGpA)

pPJV7557 was cut with Sal1 and BamH1 to isolate an insert fragment consisting of the CMV promoter through intron regions. pPJV7496 was cut with Sal1 and BamH1 to prepare a vector fragment into which the insert fragment was ligated, resulting in pPJV7558.

(d) pPJV7579 (HBsAg, CMV(cK intron), HbsAg 3′ UTR, HbsAg 5′ UTR and RBGpA)

pPJV7558 was cut with Sal1 and BamH1 to isolate an insert fragment consisting of the CMV promoter through intron regions. pPJV7496 was cut with Sal1 and BamH1 to prepare a vector fragment into which the insert fragment was ligated, resulting in pPJV7579.

### Example 9

### In Vitro Analysis of Antigen Expression by HBsAg Vector Panels

On day one, SCC15 (ATCC) or B16 (origin unknown, versions available at ATCC) cells were plated on 6 well tissue culture plates at 20-40% confluency, and allowed to grow overnight in an incubator. The host cells were propagated in media recommended by ATCC.

On day two, the transfection reaction was performed. For each vector to be tested, 20 μl of Lipofectin® reagent (Life Technologies Inc, Grand Island, N.Y.) was added to 180 μl of Optimem® media (Life Technologies, Grand Island, N.Y.), and allowed to incubate at room temperature for 45 minutes. For each vector to be tested, 2 μg of vector was mixed into 200 μl of Optimem® at 40 minutes. At 45 minutes, the vector and Lipofectin® solutions were mixed together and allowed to sit at room temperature for an additional 10 minutes. During this final incubation, the plated host cells were removed from the incubator and washed twice with Optimem® media. At 10 minutes, 1.6 ml of Optimem® was added to the Lipofectin®/vector mix, and 1 ml of the resultant mix was added to each of two cell wells. The host cells were returned to the incubator and allowed to sit undisturbed for 5 hours, at which point the Lipofectin®/vector mix was removed and replaced by standard cell maintenance media.

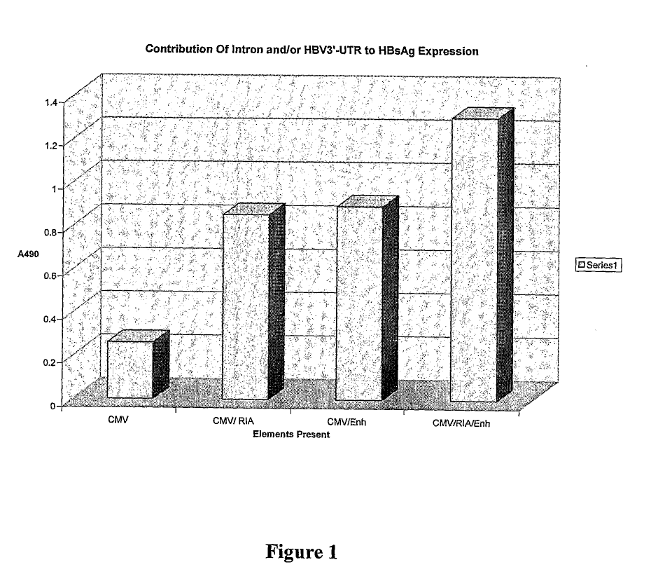

At 18 to 24 hours after the media change, from 50 to 100 μl of cell maintenance media was removed from the tissue culture plates and analyzed for antigen expression by placing the samples into reaction vessels provided in the AUSZYME® Monoclonal Diagnostic Kit (Abbott Laboratories, Abbott Park, Ill.). The volume of the test samples was brought to a volume of 200 μl with PBS, then 50 μl of conjugate and a reaction bead were added to each sample. The vessel is incubated for 80 minutes at 40° C., after which the wells were washed clean of all liquid reaction components. The beads were transferred to new tubes after which 300 μl of color development buffer was added. At 30 minutes, the color development reaction was stopped by the addition of 1M sulfuric acid, and the absorbance of the reaction was measured at 490 nm. The data shown in FIG. 1 is the average absorbance readings of the duplicate wells from two experiments.

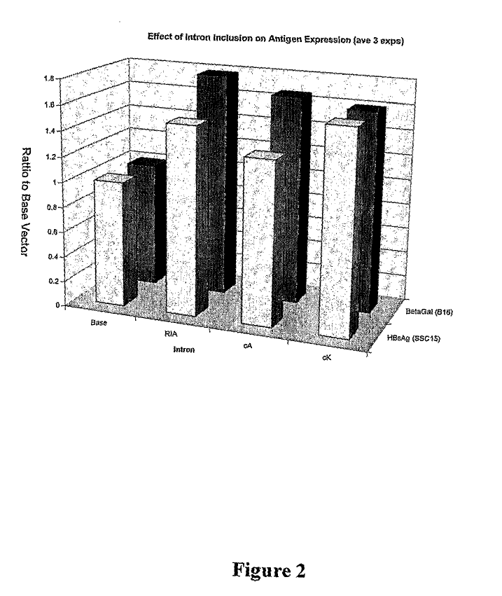

As shown in FIG. 1, the addition of RIA, the HBV 3′ UTR or both elements to a base vector (CMV promoter, exon and polyadenylation region) increased the expression of HBsAg in SCC15 cells. As shown in FIG. 2, the addition of either the chicken keratin or the chicken cardiac actin intron to a base vector (CMV promoter, exon, HBV 3′ UTR and polyadenylation region) increased expression of HBsAg in SCC15 cells.

### Example 10

### In Vitro Analysis of Antigen Expression by Beta-Gal Vector Panels

SSC-15 or B16 host cells were transfected as described in Example 9.

At eighteen to forty hours after the media change, the media supernatants were removed and the cells were washed with PBS. After removal of the wash, the cells were lysed by incubating the cells in 500 μl lysis buffer (50 mM NaPO₄, 0.1% Triton X-100, pH 7) for 5 minutes, followed by physically scraping the cells off the plastic dish. The lysates were microfuged for two minutes to remove cell debris, and 10 to 250 of the cleared lysate were added to 500 μl of reaction buffer (80 μg/ml o-nitrophenyl galactopyranoside, 50 mM NaPO₄, pH 7) and incubated at 37° C. for 10 to 20 minutes. The reaction was stopped by the addition of 500 μl of 1M Na₂CO₃ and read at 405 nm. Data is presented as the ratio of the expression of enhanced (containing an intron, HBVenh, or both) vector to a base vector.

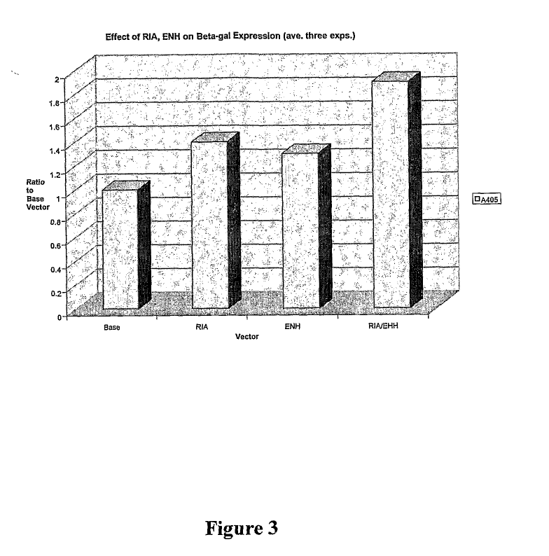

The addition of RIA, the HBV 3′ UTR or both elements to a base vector (CMV promoter, exon and polyadenylation region) increased the expression of beta-gal in both cell lines. The results for SCC15 cells are shown in FIG. 3. Addition of either the chicken keratin or the chicken cardiac actin intron to a base vector (CMV promoter, exon, the HBV 3′ UTR and polyadenylation region) increased the expression of beta-gal in both cell lines. Results for B16 cells are shown in FIG. 2.

### Example 11

### In Vitro Analysis of Antigen Expression by HSV gD Vector Panel

SCC15 or B16 host cells were transfected as described in Example 9. Eighteen hours post transfection, plates were placed on ice for 15 minutes. Each well was then washed with 2 ml of PBS (Biowhittaker, Walkerville, Md.). Cells were fixed with 0.05% gluteraldehyde (Polysciences Inc, Warrington, Pa.) diluted in PBS and incubated for 30 minutes at room temperature. All subsequent incubations lasted 1 hour at room temperature and washes between each incubation were as stated above. The plates were blocked with 2 ml of 5% dry milk (Bio Rad Laboratories, Melville, N.Y.) in PBS. Incubations with 1 ml of a 1:1000 dilution of anti-gD monoclonal (ABI, Columbia, Md.) in 2% thy milk/PBS/0.05% Tween-20® (Sigma, St. Louis, Mo.) and 1 ml of a 1:2500 dilution of goat anti-mouse HRP (KPL, Gaithersburg, Md.) in PBS/0.1% Tween-20® followed. Color was developed using 1 ml of TMB microwell substrate (BioFX, Owings Mills, Md.). The reactions were stopped with 1M H₂SO₄, the liquid was transferred to plastic cuvettes and the optical density read at 450 nm. Data is presented as the ratio of the expression of enhanced (containing an intron, HBVenh, or both) vector to a base vector.

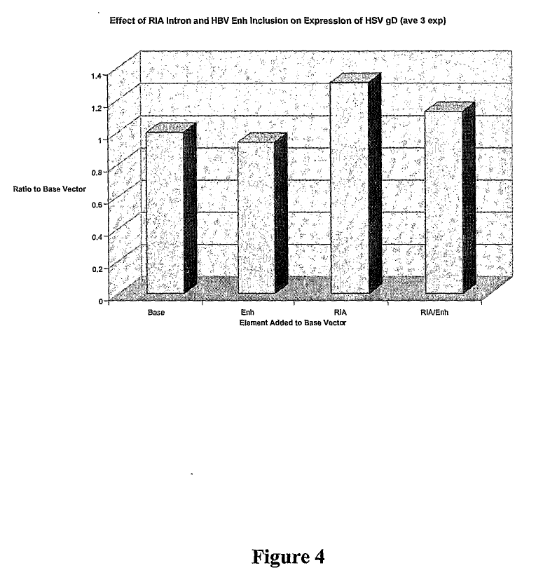

Addition of RIA with or without the HBV 3′ UTR to a base vector (CMV promoter, exon and polyadenylation region) increased the expression of HSV gD in both cell lines. Results for SC15 cells are shown in FIG. 4.

### Example 12

### In Vitro Analysis of Antigen Expression by SEAP Vector Panels

SCC15 or B16 host cells were transfected as described in Example 9. At eighteen to forty hours after the media change, the media supernatants were removed and heated at 70° C. for 30 minutes. 10 to 25 μl of the heat-inactivated supernatants were incubated for 5 minutes with 1/10^{th} volume of 100 mM 1-homoarginine. 500 μl of alkaline phosphatase reaction buffer (cat #172-1063, Bio-Rad, prepared according to instructions) were added to the lysates and incubated at 37° C. for 10 to 20 minutes. The reaction was stopped by the addition of 500 μl of 1M NaOH and read at 405 nm. Data is presented as the ratio of the expression of enhanced (containing an intron, HBVenh, or both) vector to a base vector, or the ratio of the expression of the experimental signal peptides to the human TPA signal peptide vector.

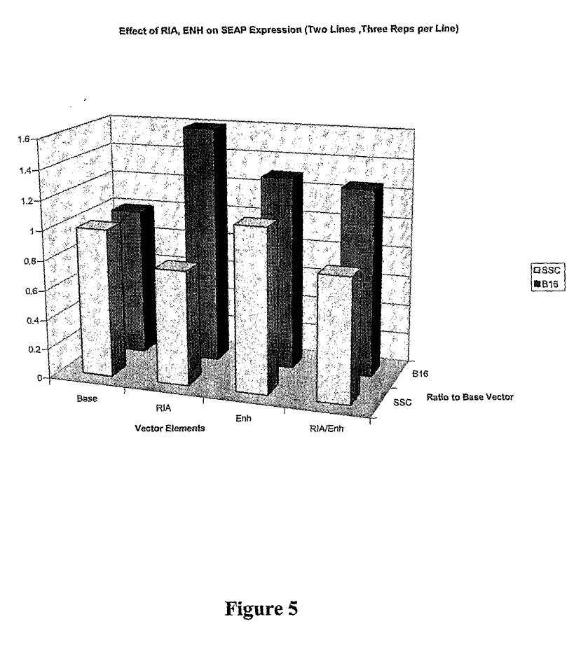

As shown in FIG. 5, the addition of RIA, the HBV 3′ UTR or both elements to a base vector (CMV promoter, exon and polyadenylation region) increased the expression of SEAP in B16 cells. Unexpectedly, only the addition of the HBV 3′ UTR to a base vector (CMV promoter, exon and polyadenylation region) increased the expression of SEAP in SCC15 cells.

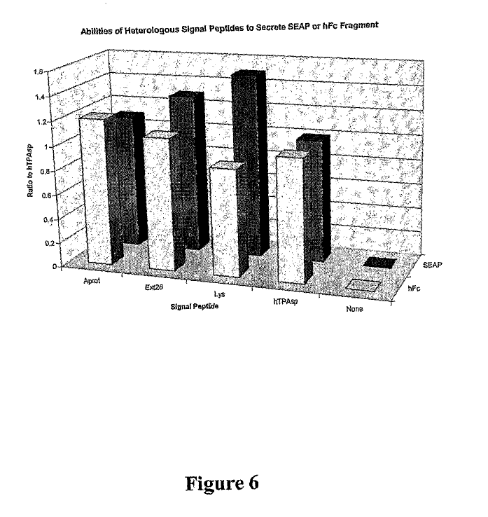

Addition of signal peptides from either bovine aprotinin, chicken lysozyme, or tobacco extensin to the N-terminus of mature SEAP allowed for efficient secretion of SEAP into cell media supernatants of both cell lines. Results for B16 cells are shown in FIG. 6.

### Example 13

### In Vitro Analysis of Antigen Expression by Human IgG Fc Fragment Signal Peptide Panel

SCC15 or B16 host cells were transfected as described in Example 9. The media supernatants were removed from eighteen to forty hours after the media change.

ELISA plates (Costar) were incubated overnight at 4° C. with 100 μl of goat anti-human IgG (Sigma #I3382, 1/1000 dilution in carbonate coating buffer) per well. All subsequent incubations lasted 1 hour at room temperature with washes (10 mM Tris, 150 mM NaCl, 0.1% Brij-35, pH8.0) between each incubation. The wells were then blocked with 1000 of 5% dry in PBS, followed by incubation with serially diluted media supernatants in dilution buffer (2% dry milk, PBS, 0.05% Tween-20®). This was followed by incubation with 100 μl of goat anti-human IgG/HRP (Sigma #A6029, 1/5000 dilution in dilution buffer) per well, followed by color development using 1000 of TMB microwell substrate. The reactions were stopped with 100 μl of 1M H₂SO₄, and read at 450 nm. Data is presented as the ratio of the expression of the experimental signal peptides to the human TPA signal peptide vector.

Addition of signal peptides from either bovine aprotinin, chicken lysozyme, or tobacco extensin to the N-terminus of the human Fc fragment allowed for efficient secretion of hFc into cell media supernatants of both cell lines. Results for B 16 cells are shown in FIG. 6.

### Example 14

### Use of the HBsAg, HSVgD and Flu-M2 Plasmid Expression Vectors for Immunisation of Mice

(a) Preparation of Cartridges of Immunization

For each plasmid to be tested, 25 mg of 2 micron gold powder was weighed into a microfuge tube. After the addition of a 250 μl aliquot of 50 mM spermidine (Aldrich Chemical, Inc, Milwaukee, Wis.), the tube was vortexed and briefly sonicated. The gold was microfuged out, and the spermidine replaced by a fresh 100 μl aliquot. The gold was resuspended by vortexing, after which 25 μg of DNA was added to the tube and mixed. While the tube was lightly vortexed, 100 μl of 10% CaCl₂ (Fujisawa USA, Inc, Deerfield, Ill.) was added to precipitate the DNA onto the gold beads. The precipitation reaction was allowed to proceed for 10 minutes on the benchtop, after which the gold was collected by a brief microfuge spin and washed three times with absolute ethanol (Spectrum Quality Products, Inc, Gardena, Calif.) to remove excess precipitation reagents. The washed gold/DNA complex was then resuspended in 3.6 ml of 0.05 mg/ml polyvinylpyrrolidone (360 KD, Spectrum Quality Products, Inc, Gardena, Calif.) in absolute ethanol. This slurry was then injected into a Tefzela tube (McMaster-Carr, Chicago, Ill.) located in a tube turner (PowderJect Vaccines) which coats the inside of the Tefzela tube with the gold/DNA complex. After the tube turning procedure was completed, the tube was cut into 0.5″ “shots” of vaccine which were loaded into the XR1 device (PowderJect Vaccines) for delivery to the mice.

(b) Vaccination Procedure

Four to six week old mice were anesthetized with a mixture of Ketasetâ (Fort Dodge) and Rompunâ (Bayer). The bellies were shaved with a pair of electric clippers to remove hair, and two non-overlapping “shots” of vaccine were delivered via the XR1 device (450 psi) to the shaved area. The animals were returned to their cages and bled at six weeks post vaccination. Balb/c mice were used to evaluate the HBsAg expression vectors, and Swiss Webster mice were used to evaluate the HSV-gD and Flu M2 expression vectors.

### Analysis of Sera for Anti-HBsAg Antibodies

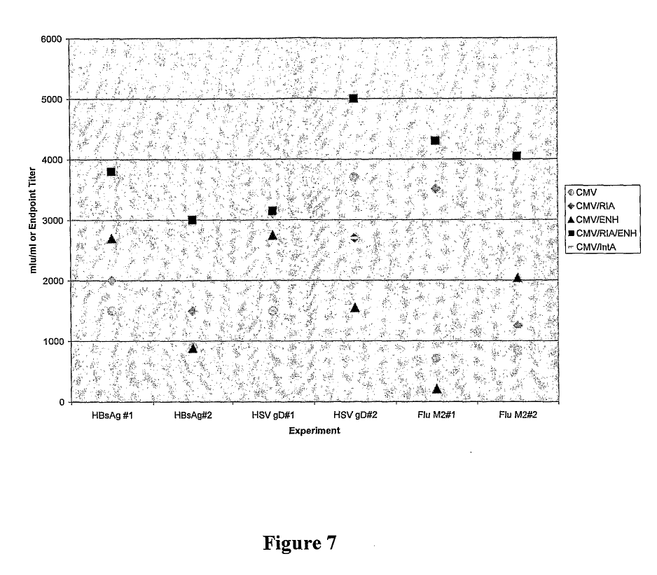

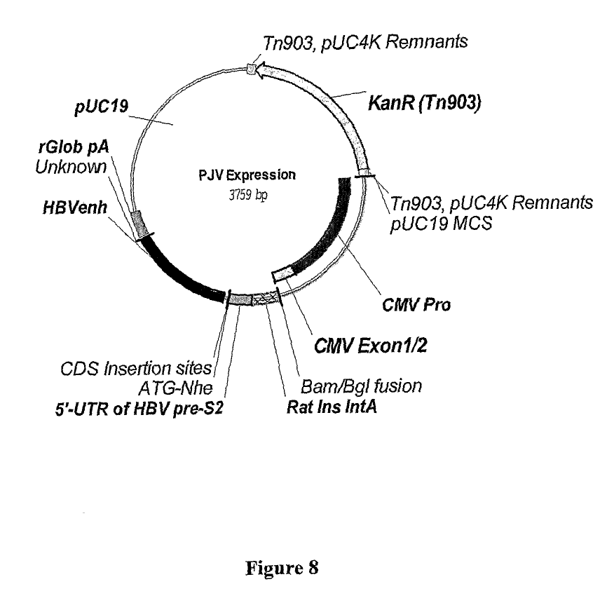

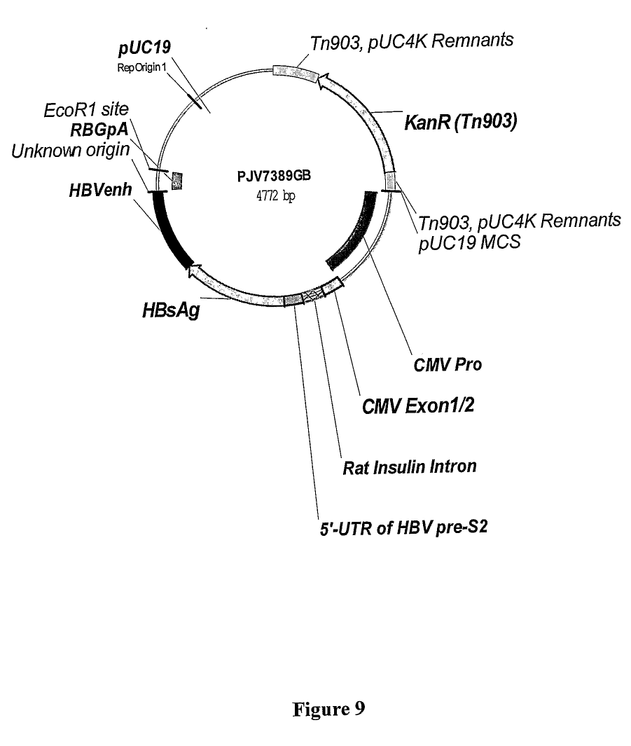

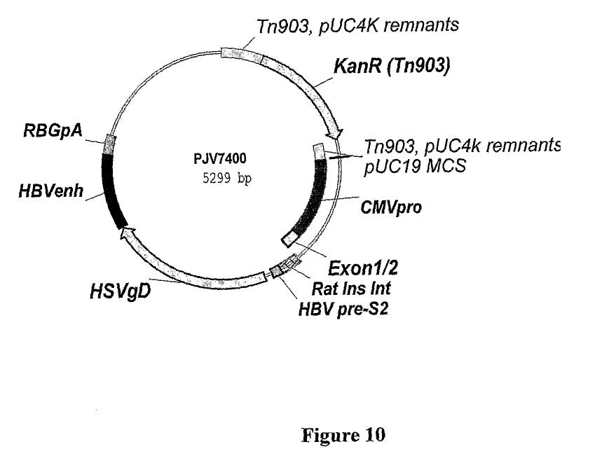

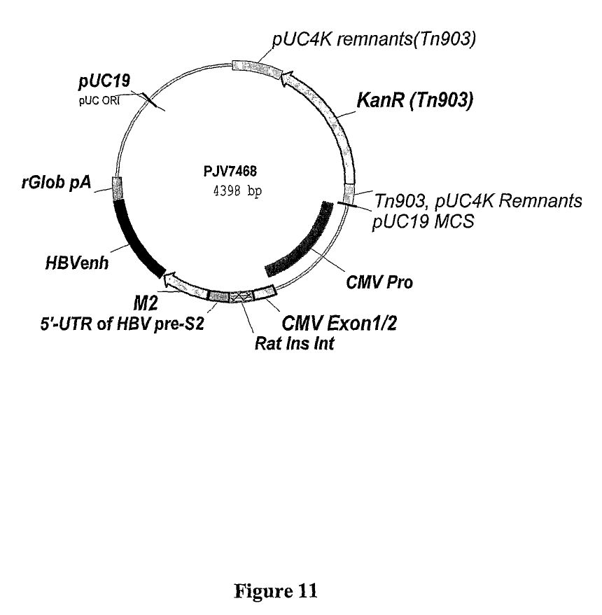

At six weeks, blood samples were harvested from vaccinated animals. A volume of serum isolated from these samples was placed into wells of a reaction vessel supplied with the AUSAB® EIA Diagnostic Kit (Abbott Laboratories, Abbott Park, Ill.). The volume of sera added depended upon the antibody titer of the sample, and the sample was diluted with sample dilution buffer to fall within values obtainable with a quantification panel. 200 μl from each vial of the AUSAB® Quantification Panel (Abbott Laboratories, Abbott Park, Ill.) was added to wells of the reaction vessel. To each well a bead was added, after which the vessel was sealed and incubated for two hours at 40° C. The wells were then washed of all liquid reaction components. To each washed well was added 200 μl of conjugate mix, after which the vessel was sealed and incubated for two hours at 40° C. The wells were then washed of all liquid reaction components. The beads were transferred to new tubes after which 300 μl of color development buffer was added. At 30 minutes, the color development reaction was stopped by the addition of 1M sulfuric acid, and the absorbance of the reactions was measured at 490 nm in a Quantum II® spectrophotometer (Abbott Laboratories, Abbott Park, Ill.). This spectrophotometer calculates the antibody levels of a sample by comparing the absorbance of the sample with a standard curve generated with the quantification panel. These antibody levels were then corrected for dilution factors. The data shown in FIG. 7 are the geometric mean titers of all animals vaccinated with a particular vector.

### Analysis of Sera for Anti-Flu M2 Antigen Antibodies

96-well Costar medium-binding ELISA plates (Fisher Scientific, Pittsburgh, Pa.) were coated with a synthetic Flu M2 peptide (QCB/Biosource, Hopkinton, Mass.) at a concentration of 1 μg/ml in PBS (Biowhittaker, Walkerville, Md.) and incubated overnight at 4° C. The plates were washed three times with 10 mM Tris (Sigma, St. Louis, Mo.)/150 mM NaCl (Fisher Scientific)/0.1% Brij-35 (Sigma), then blocked with 5% dry milk (Bio Rad Laboratories, Melville, N.Y.) in PBS for 1 hour at room temperature. All subsequent incubations were at room temperature for one hour and washes between each incubation were as stated above. Sample mouse sera, a standard (high titer, anti-M2 mouse sera) and a negative control (anti-HBsAg mouse sera) were diluted in 2% dry milk/PBS/0.05% Tween-20® (Sigma) and incubated in the ELISA plates. Goat anti-mouse IgG (H+L) biotin conjugated antibody (Southern Biotechnology Associate, Birmingham, Ala.) diluted 1:8000 in 2% dry milk/PBS/0.05% Tween-20® and streptavidin-horseradish peroxidase conjugate (Southern Biotechnology) diluted 1:8000 in PBS/0.1% Tween-20 followed. Color was developed using TMB substrate (BioFX, Owings Mills, Md.). The reactions were stopped with 1M H₂SO₄ and the plates read at 450 nm with an Emax precision microplate reader (Molecular Devices, Sunnyvale, Calif.). SoftMax Pro 4.1 software (Molecular Devices) was used to calculate endpoint titers using a four-parameter analysis. Titers were normalized to the standard serum, which had a pre-determined titer, to minimize assay-to-assay and plate-to-plate variation. Results are shown in FIG. 7.

### Analysis of Sera for Anti-HSV gD Antigen Antibodies

96-well Costar medium-binding ELISA plates (Fisher Scientific, Pittsburgh, Pa.) were coated with HSV gD (Viral Therapeutics, Ithaca, N.Y.) protein at a concentration of 1 ug/ml in PBS (Biowhittaker, Walkerville, Md.) and incubated overnight at 4° C. The plates were washed three times with 10 mM Tris (Sigma, St. Louis, Mo.)/150 mM NaCl (Fisher Scientific)/0.1% Brij-35 (Sigma), then blocked with 5% dry milk (Bio Rad Laboratories, Melville, N.Y.) in PBS for 1 hour at room temperature. All subsequent incubations were at room temperature for one hour and washes between each incubation were as stated above. Sample mouse sera, a standard (high titer, anti-gD mouse sera) and a negative control (anti HBsAg mouse sera) were diluted in 2% dry milk/PBS/0.05% Tween-20 (Sigma) and incubated in the ELISA plates. Goat anti-mouse IgG (H+L) biotin conjugated antibody (Southern Biotechnology Associate, Birmingham, Ala.) diluted 1:8000 in 2% dry milk/PBS/0.05% Tween-20 and streptavidin-horseradish peroxidase conjugate (Southern Biotechnology) diluted 1:8000 in PBS/0.1% Tween-20 followed. Color was developed using TMB substrate (BioFX, Owings Mills, Md.). The reactions 25, were stopped with 1M H₂SO₄ and the plates read at 450 nm with an Emax precision microplate reader (Molecular Devices, Sunnyvale, Calif.). SoftMax Pro 4.1 software (Molecular Devices) was used to calculate endpoint titers using a four-parameter analysis. Titers were normalized to the standard serum, which had a pre-determined titer, to minimize assay-to-assay and plate-to-plate variation. Results are shown in FIG. 7.

### Example 15

### Construction of Plasmid pPJV1671, Influenza DNA Vaccine Vector

Plasmid pPJV1671 was constructed which encodes and can express the Hemagglutinin (HA) antigen of influenza A/Panama/2007/99 (H3N2).

Construction of pPJV1671 is best described in three major stages:
(i) Cloning the expression gene;
(ii) Engineering the vector backbone; and
(iii) Engineering the final plasmid.

Cloning the Expression Gene

The coding sequence for influenza HA was obtained by a standard reverse transcriptase-polymerase chain reaction (RT-PCR) cloning technique using a sample of A/Panama/2007/99 virus obtained from the Centers for Disease Control and Prevention (Atlanta, Ga.) as a source of template ribonucleic acid (RNA).

The following steps were employed in cloning the expression gene, HA:
RT-PCR production of dsDNA fragment of RNA segment 4 of A/Panama/2007/99 (H3N2);
propagation of RNA segment 4 DNA clone in a standard pUC19-based vector in E. coli;
sequence analysis of the H3 Panama HA coding sequence within the RNA segment 4 clone; and
a second PCR reaction to generate a DNA fragment containing the H3

Panama HA coding sequence (without its ATG codon) with ends compatible with the pPJV7563 DNA vaccine “empty” vector (Nhe I and Bsp 120I).

Engineering the Vector Backbone, pPJV7563

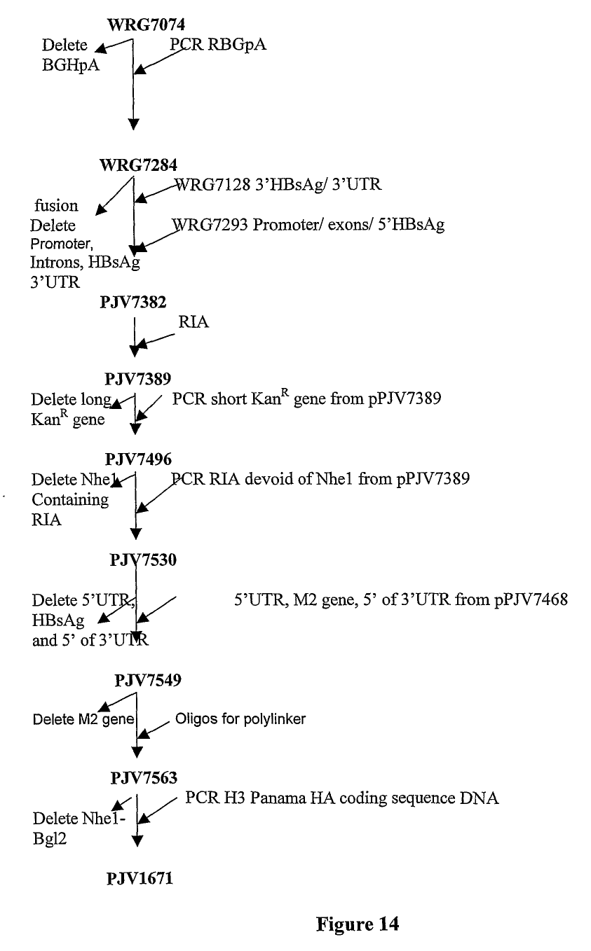

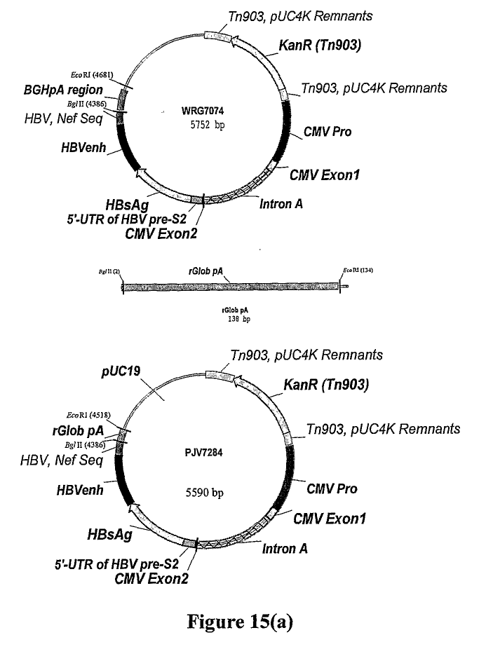

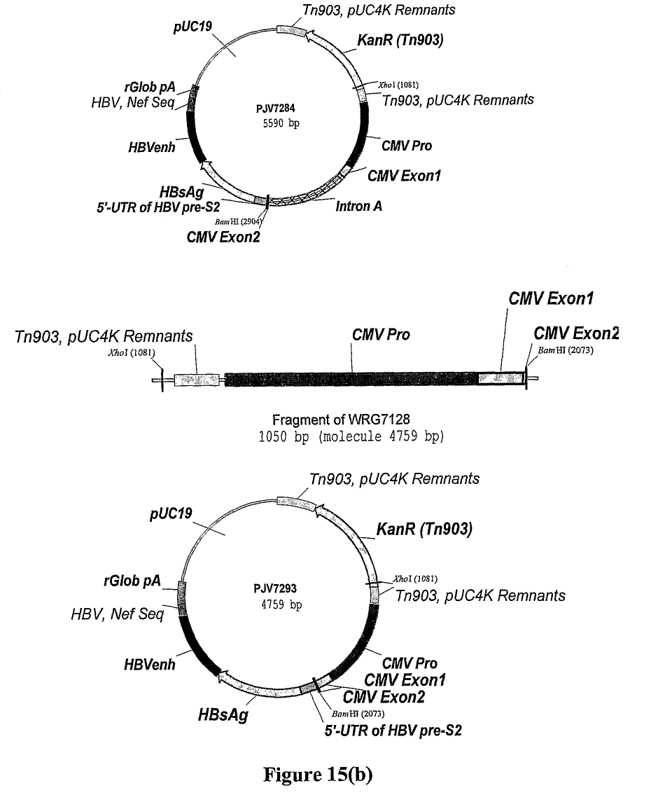

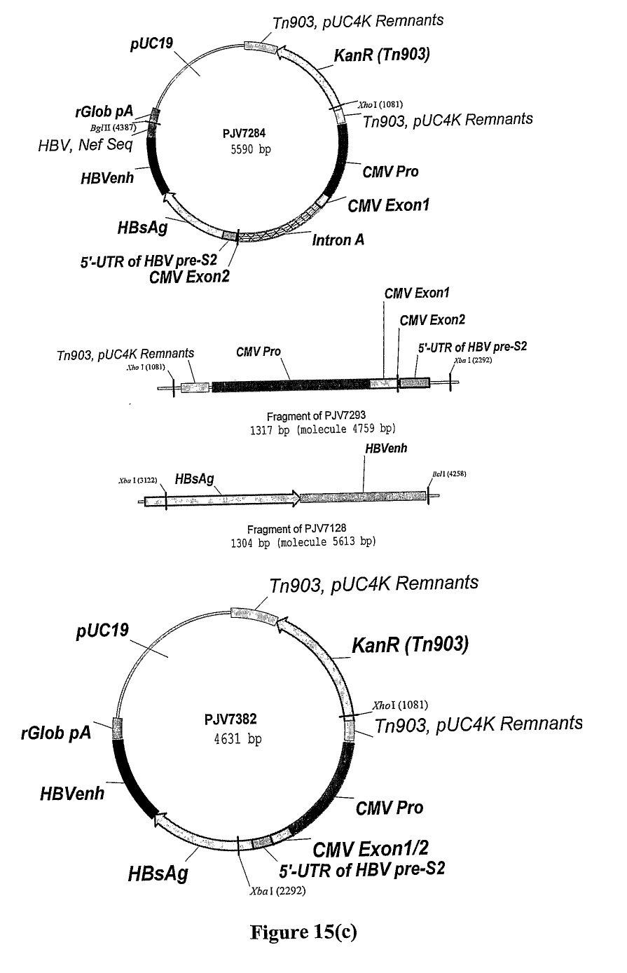

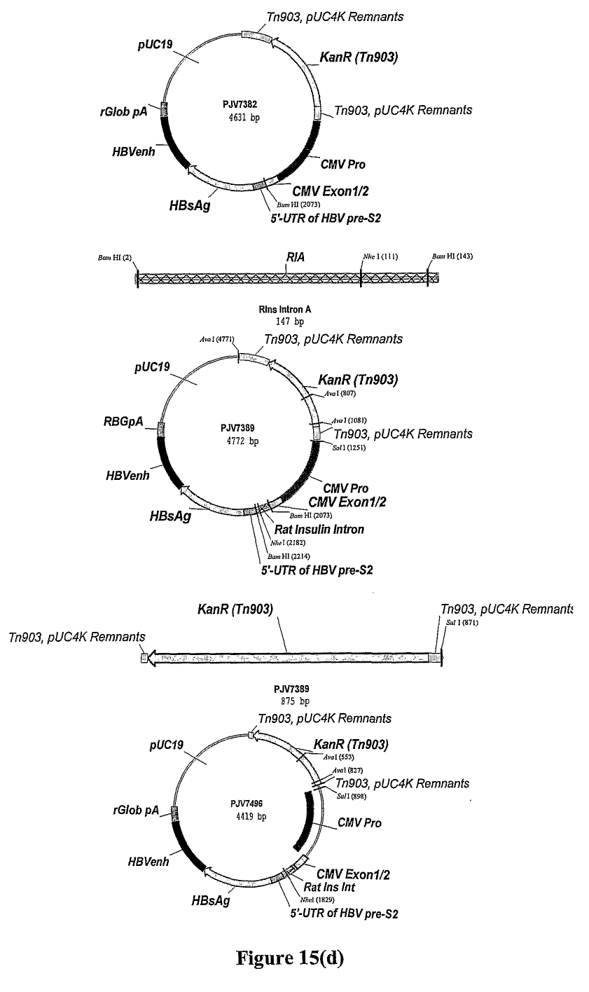

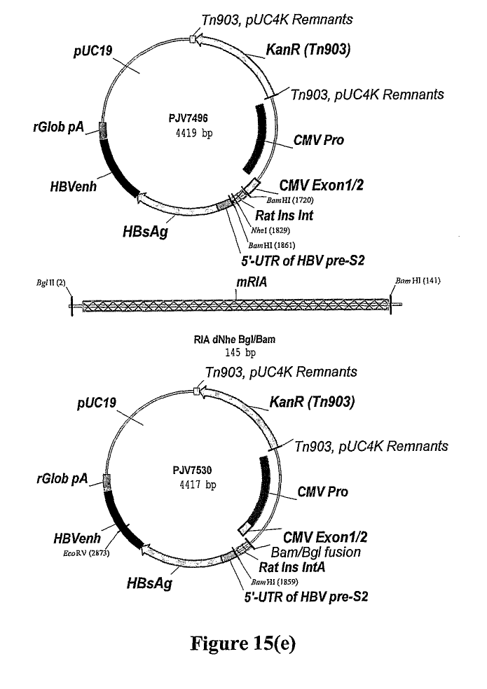

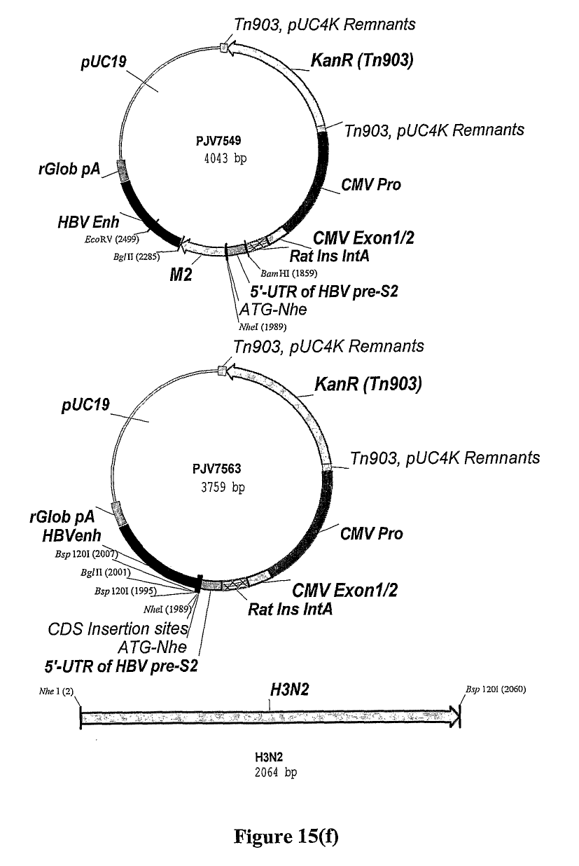

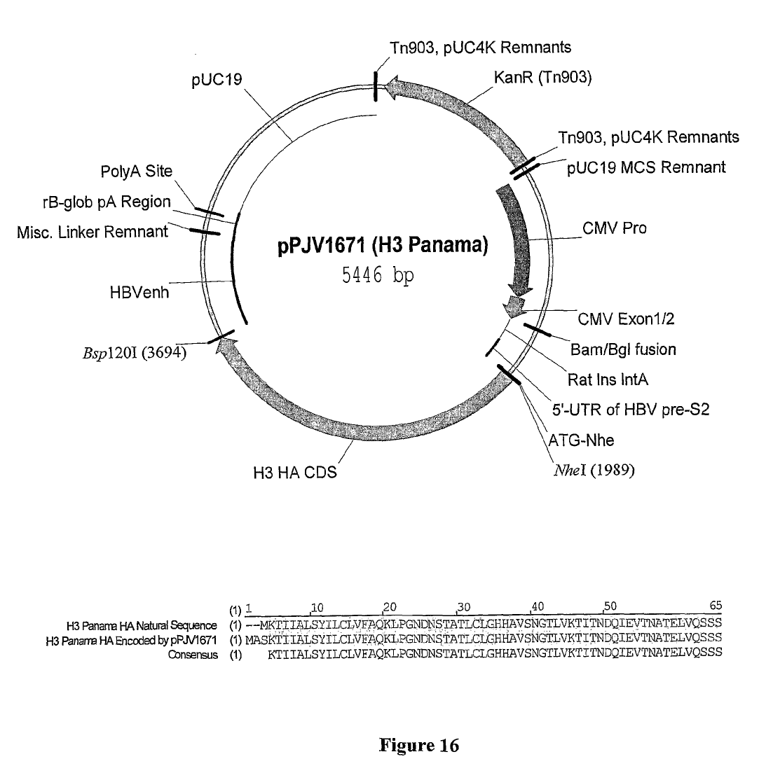

The plasmid backbone for pPJV1671 is pPJV7563. The majority of the plasmid backbone sequences in pPJV7563 are also found in pWRG7128, a DNA vaccine vector that has been evaluated in several human clinical trials. This section provides a synopsis of the construction of pPJV7563. A detailed flowchart is provided in FIG. 14 of the construction of pPJV7563 and pPJV1671, and a tabular comparison below (Table 2) of key elements in plasmids pWRG7128 and pPJV1671 is provided. The map of pPJV1671 is shown in FIG. 16.

TABLE 2
Comparison of Key Elements in Vector Backbones: Plasmids
WRG7128 (HBsAg) and pPJV1671 (Influenza HA)

| | | Biological Effect or |
|---|---|---|
| | Reason or Purpose for the | Significance or Possible |
| Description of Change | Change | Effect of the Change |
| The kanamycin resistance | To remove extraneous | No negative change to |
| gene fragment in pPJV1671 | sequences from vector | vaccine effectiveness or |
| is 354 bases smaller than | | safety foreseen |
| that used in pWRG7128. | | |
| The Sph1-Pst1 linker | To remove extraneous | No negative change to |
| upstream of the CMV | sequences from vector. | vaccine effectiveness or |
| promoter in pWRG7128 | Allows these restriction | safety foreseen |
| was removed. | sites to be more effectively | |
| | utilized in constructing | |
| | future vaccines. | |
| The intron A from CMV | The CMV exons were fused | Fusion of the CMV exons |
| was removed and in | to recreate 5′-UTR of | should not change vaccine |
| pPJV1671, the CMV exon1 | spliced pWRG7128 | effectiveness or safety |
| has been fused to the | transcript. | profile. |
| remaining 9 bases of CMV | | |
| exon2. | | |
| The intron A from the rat | To replace the CMV intron | The addition of the rat |
| insulin gene has been | A sequence with a | insulin intron A to vaccine |
| inserted between the fused | functional alternative. | vectors has been shown to |
| CMV exons and the 5′- | | increase antigen expression |
| UTR of HBsAg. | | and subsequent antibody |
| | | responses. |
| The polyadenylation region | To employ an alternative | No negative change to |
| has been changed from | polyadenylation signal | vaccine effectiveness or |
| bovine growth hormone to | | safety foreseen. |
| rabbit beta-globin. | | |

Engineering the Final Plasmid, pPJV11671

Two major steps were involved in engineering the final plasmid, pPJV1671, from the backbone, pPJV7563 (as illustrated in FIG. 14) these are:
deletion of the Nhe1-Bgl II site from pPJV7563; and
insertion of the H3 Panama HA coding sequence into pPJV7563 yielding the final pPJV1671, H3 Panama HA DNA vaccine vector.

A complete sequence analysis of pPJV1671 confirmed all vector backbone and HA coding sequences that are listed below in Table 3.

Comparison of pWRG7128 and pPJV1671

The major differences in the vector backbones between the clinically tested pWRG7128 encoding HBsAg and the influenza vaccine pPJV1671 vector encoding H3 Panama HA are shown above in Table 2. These include use of the rat insulin intron A in place of the human Cytomegalovirus (hCMV) intron A element and use of the rabbit (3-globin polyadenylation sequence in place of the bovine growth hormone polyadenylation sequence. Extensive animal studies with pPJV1671 described have demonstrated good influenza DNA vaccine performance in both small and large animals. Plasmid pPJV1671 also performs well in humans following PMED of this DNA vaccine as discussed below in Example 16.

Feature and Functional Map of pPJV1671

The functional map of pPJV1671 is shown in FIG. 16. The features of this map are listed in Table 3. Plasmid pPJV1671 employs the hCMV immediate early promoter and 5′ noncoding sequences from exons 1 and 2. This promoter is linked to the rat insulin intron A and the 5′ UTR of the HBV pre-S2 gene. Translation of the H3 Panama HA coding sequence begins at an ATG codon that is fixed in the vector in the context of a consensus Kozak translation initiation sequence. The HA coding sequence is followed by a transcriptional enhancer of HBV (HBVenh). Finally, transcription termination is facilitated by a rabbit β-globin polyadenylation site.

Because the natural ATG translation initiation codon of the HA gene of A/Panama/2007/99 (H3N2) does not conform to the Kozak consensus sequence for translation initiation, it was elected to use the ATG element supplied by pPJV7563 DNA vaccine vector for translation initiation. Antigen expression from DNA vaccine vectors is enhanced using ATG codons that conform to the Kozak consensus. Use of the vector-supplied ATG codon (via insertion at the Nhe I site) results in a minor 2-amino acid insertion at the amino terminus of the coding sequence of the HA gene as depicted in FIG. 16.

Origin of Nucleotide Sequences in pPJV1671

The detailed construction history and sequencing reports for pNV1671 allows for assignment of the origin of all nucleotide positions within this plasmid. BLAST alignments between vector component sequences and the GenBank database were performed to verify that correct component assignments have indeed been made.

TABLE 3
Identification of Components Comprising Plasmid pPJV1671

| Base Numbers | Component Identification |
|---|---|
| 1-896 | Kanamycin resistance gene, flanked by tn903 |
| | sequences (Tn903, pUC4K remanants 1-44 and |
| | 861-896) |
| 897-902 | Sal 1 cloning site/pUC 19 MCS |
| 903-1587 | CMV promoter |
| 1588-1718 | CMV exon 1/2 fusion |
| 1719-1724 | BamH1/Bgl2 fusion |
| 1725-1857 | Rat insulin intron A |
| 1858-1863 | BamH1 cloning site |
| 1864-1984 | Non-coding preS2 region of HbsAg |
| 1985-1987 | ATG initiation codon |
| 1988-1993 | Nhe1 cloning site |
| 1994-3688 | H3N2 HA coding sequence |
| 3689-3691 | Stop codon |
| 3692 | G nucleotide from H3 Panama 3′ UTR |
| 3693-3698 | Bsp 120I cloning site |
| 3699-4231 | HBV enhancer |
| 4232-4242 | Cloning artifact. Unknown origin |
| 4243-4373 | Rabbit beta-globin polyadenylation region |
| 4374-4379 | EcoR1 cloning site |
| 4380-5446 | PUC19 vector |

Sequence of pPJV1671 (Master Cell Bank)

Sequencing was performed by the PowderJect Research Department using Qiagen Megaprep DNA prepared from the newly constructed pPJV1671 plasmid. Actual sequence data were in 100% agreement with the theoretical sequence predicted for pPJV1671. Sequencing during the manufacturing process also showed that the plasmid sequence was also in 100% agreement with the theoretical and research sequences. The various plasmid elements and positions in the sequence are provided in Table 3 above.

### Example 16

### Nonclinical Evaluation of pPJV1671: Immunogenicity of the Monovalent Influenza DNA Vaccine pPJV1671 in a Large Animal Model

The domestic pig model was used to investigate the immunogenicity of pPJV1671 the generation of which is described above in Example 15. Eight-week old domestic white pigs (barrows and gilts) were utilized in this study. Hemagluttination inhibition (HI) antibody titers were measured for candidate study animals and were <1:10 for all animals entered in the study.

Two groups of 8 animals each were vaccinated by PMED of the pPJV1671 DNA vaccine. Both groups received primary and booster immunizations spaced 4 weeks apart. Each immunization consisted of two side-by-side administrations to skin. The DNA vaccine dose for each administration was 1 μg DNA condensed onto the surface of 0.5 mg gold particles. The DNA vaccine was delivered with the XR-1 clinical and research devices operated at 500 psi helium pressure.

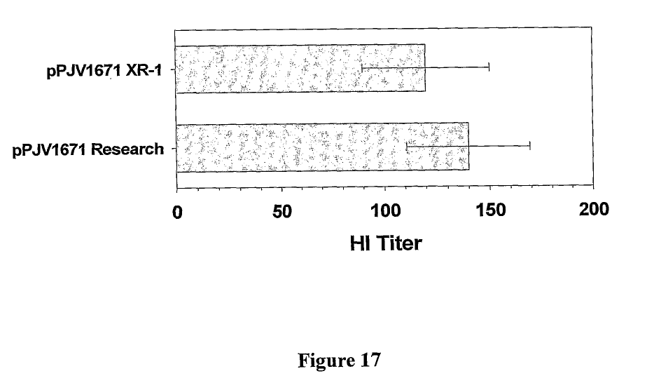

Pigs, were vaccinated on Day 0, boosted on Day 28 and bled on Day 42, two weeks after the booster vaccination. Sera were tested for HI antibody responses using a standard HI assay. Results of this study are shown in FIG. 17 and demonstrate that the pPJV1671 DNA vaccine induced significant HI antibody titers in 100% of the test animals with mean HI antibody titers well in excess of the 1:40 surrogate HI antibody titer for protection against influenza in humans.

### Example 17

### Clinical Trial Assessing Influenza DNA Vaccine Construct in Humans

### Introduction

A phase I dose escalating clinical study was carried out to evaluate safety and immunogenicity of the pPJV1671 DNA vaccine which is a monovalent PMED influenza DNA vaccine containing the HA (hemagglutinin) gene from A/Panama/2007/99 (H3N2). Safety was assessed by monitoring local and systemic adverse events from vaccination until study completion. Immunogenicity was assessed by measuring serum hemagglutination-inhibiting (HI) antibody levels.

Three groups of 12 healthy adult subjects received a single dose on day 0 of either 1, 2 or 4 μg of DNA vaccine, delivered as 1, 2 or 4 PMED administrations. The PMED influenza DNA vaccine elicited serum hemagglutination-inhibition (HI) antibody responses at all 3 dose levels, with the highest and most consistent responses in subjects vaccinated with the highest dose level. Antibody responses were greatest at the last time point tested, day 56. Treatment-related reactogenicity was limited to mild to moderate skin reactions at the vaccine site, which were primarily self-limiting. These results provide an indication of the safety and immunogenicity of the DNA vaccine for influenza generated.

### Materials and Methods

### Vaccine and Delivery System

For the clinical plasmid, the HA coding sequence was obtained by a standard reverse transcriptase-polymerase chain reaction (RT-PCR) cloning technique using a sample of A/Panama/2007/99 virus as a source of template RNA (virus was a gift of J. Katz, Centers for Disease Control and Prevention). The H3 Panama HA coding sequence was inserted into the pPJV7563 vector as described above in Example 15 yielding the final pPJV1671H3 Panama HA DNA vaccine vector. Complete sequence analysis of pPJV1671 confirmed all vector backbone and HA coding sequences.

Plasmid pPJV1671 was manufactured under good manufacturing practices at Strathman GmbH (Hannover, Germany). The plasmid DNA was coated on 1-3 μm gold particles, and formulated for PMED using the PowderJect XR-1 device, using previously described methods (Roy et al., (2001) Vaccine 19:764-78). Each dose contained a nominal value of 1 μg DNA coated on 0.5 mg gold. Vaccine quality parameters analyzed included quantity of gold and DNA, in vitro expression, immunopotency in mice, and absence of bioburden and endotoxin.

### Patient Population and Setting

The clinical phase of the study was conducted at MDS Pharma Services, Lincoln, Nebr., and was approved by the local Institutional Review Board. Thirty-six (36) adult volunteers were enrolled (Table 4).

TABLE 4
Demographics of the study population

| | Group 1 | Group 2 | Group 3 |
|---|---|---|---|
| Number per group | 12 | 12 | 12 |
| Mean age (years) | 31 | 31 | 32 |
| Age range (years) | 20-48 | 20-50 | 21-49 |
| Male:Female | 7:5 | 4:8 | 5:7 |
| Prevaccination | 16 (5-40) | 17 (5-40) | 12 (5-40) |
| HAI GMT (range) | | | |

Subjects were considered eligible if they were healthy, age 19-50, not pregnant or lactating, able to provide written Informed Consent, and with prevaccination hemagglutination inhibition (HI) titers to influenza A/Panama of ≧10 and ≦40.

Reasons for exclusion included immunosuppressive therapy in the last 6 months, history of skin disease, scarring, moles, cuts or tattoos at the site of immunization, allergy to gold, history of chrysotherapy, receipt of influenza vaccine in the past 12 months, influenza-like symptoms or diagnosed influenza in the current season, or presence of any medical condition where influenza vaccination is not recommended.

All subjects completed the study and were evaluated for safety and immunogenicity, with the exception of two subjects. One subject was lost to follow-up and another subject was non-compliant. However, as all 36 subjects provided a pre-vaccination sample, received vaccination, and were evaluated at least once post-vaccination for safety and immunogenicity, data were available for evaluation of safety and immunogenicity on all subjects. All study procedures were in accordance with the Helsinki Declaration of 1975, with the latest amendments Edinburgh, Scotland (2000) and the International Conference on Harmonization guidelines on Good Clinical Practice (Step 4, 1 May 1996).

### Clinical Study Design

Subjects were assigned to three treatment groups, 12 per group, on a sequential basis. Each PMED DNA vaccine administration delivered 1 μg DNA, plasmid pNV1671 (H3 Panama) on 0.5 mg gold. The first group received a single vaccine administration on the inner aspect of the upper arm on day 0, Subjects in group 2 received two vaccine immunizations on day 0, for a total of 2 μg DNA, administered to adjacent sites on the inner upper arm. Subjects in group 3 received 4 administrations on day 4, for a total of 4 μg DNA. Vaccines were administered using the PowderJect XR-1 device at a helium pressure of 500 psi, a pressure shown to be well tolerated in earlier studies (Roy et al. (2001) Supra, Rottinghaus et al. (2003) Vaccine 21(31):4604-8 and Tacket et al. (1999) Vaccines 17(22):2826-9).

### Clinical Safety and Immunogenicity Evaluations

Safety was assessed by monitoring vaccination site reactions and recording the incidence of local and systemic adverse events during the study. All subjects were evaluated for vaccine safety and local tolerability at the time of immunization, 1 hour and 2 hours post-immunization. Subjects were further evaluated for local tolerability on days 3, 7, 14, 21, 28, 56, and 180. Systemic adverse events were monitored by physical examination, vital signs, laboratory safety tests, and anti double-stranded DNA antibody testing. These tests were conducted prior to and after immunization.

Immunogenicity of each vaccine was determined by collecting blood samples on day 0 (pre-immunization), and at 14, 21, and 56 days post-immunization for determination of HAI titers against A/Panama/2007/99, using a modification of a previously described method (Kendal, et al (1982) Concepts and procedures for laboratory-based influenza surveillance. US Department of Health and Human Services, Public Health Service, Centers for Disease Control: B17-35).

### Clinical Data Analysis

Safety data were tabulated. Each HI titer was reported as the geometric mean (GMT) of two independent determinations, which usually yielded the same or similar results. A titer of 5 was assigned if the sample had a titer below 10, the detection limit of the assay. For each group, the GMT at each time point and 95% Confidence Interval (CI) were calculated using logarithmically transformed titers. For each time point, the GMT ratio from baseline for each dosage group was compared by chi-square test using procedure CATMOD in SAS. Differences in the GMT among the three groups were also compared.

Seroconversion rate (percent subjects with ≧4-fold increases in HAI titer over the pre-immunization titer), and the seroprotection rate (the proportion of subjects achieving a post-immunization titer≧40) were calculated and compared between groups by chi-square test using procedure CATMOD in SAS for comparing % responder with logit response link function.

### Results

### Safety and Reactogenicity

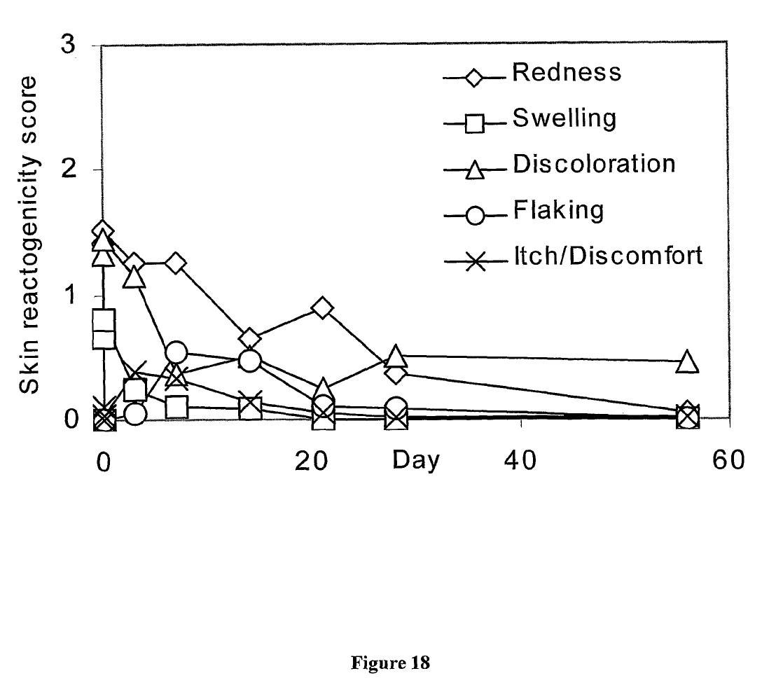

Local reactions were assessed for all 84 vaccine administration sites (FIG. 18). The local skin reaction scores from all three groups (84 total vaccination sites) were averaged. Skin reactions were scored according to the following criteria:
Erythema: 0=none; 1=red blush; 2=mild sunburn; 3=beet red.
Edema: 0=none; 1=barely thicker; 2=notably thicker; 3=large firm hive.
Discoloration: 0=none; 1=barely seen; 2=notably discolored; 3=brownish shoe polish.
Flaking: 0=none; 1=like fine white dandruff; 2=peeling like sunburn; 3=thicker, yellow, crusty.
Itch/discomfort: 0=none; 1=mildly itchy or slightly sore upon touch; 2=moderately itchy or sore

As expected based on previous studies (Roy et al. (2001), Rottinghaus et al. (2003) and Tacket et al. (1999) all supra), the subjects experienced local dermal reactions, however no local reactions in the severe (score=3) range occurred. There was no recorded bleeding or skin damage. The typical local reaction was characterized by mild to moderate erythema, edema, and skin discoloration, and occasionally itch/discomfort, followed by mild superficial skin flaking.

Infrequent local reactions included petechiae (2/84 sites), minor bruising (2/84 sites) and small scabs (15/84 sites). While edema generally resolved by 14 days post-vaccination, erythema and skin discoloration persisted through 28 days. Of the 84 total vaccination sites, mild skin discoloration was still present in 30 at 56 days and 21 at 180 days post-vaccination. One subject also had detectable bruising at 2 sites at Day 180.

Systemic adverse events observed during the study were mild and considered unrelated to the treatment. Events reported from 7 to 56 days post-immunization included headache (11 events in 10 subjects), fatigue (4 events in 3 subjects), myalgia (3 events in 1 subject), fever (2 events in 2 subjects), cold feeling in the hand (2 events in 2 subjects), back pain (2 events in 1 subject) and nausea, arthralgia, muscle tightness, shivering, intermittent hypertension, and intermittent hypotension (1 event each in 1 subject). No anti-double stranded DNA antibodies were detected. Overall the local and systemic adverse event profile for all study groups provided an indication of the safety of influenza DNA vaccine delivered by PMED.

### Antibody Responses

Subjects were pre-screened for HI titer to Influenza A/Panama of ≧10 and ≦40, yet some subjects had titers of ≦10 on the day of vaccination. Baseline HI titers were all <40, and there were no significant differences in baseline titers among groups (p=0.439; see Table 4 above).

Immunization resulted in significant rises in the geometric mean titers (GMT) at all time points in all three groups (as shown in Table 5 below).

TABLE 5
Serum antibody responses, seroconversion and seroprotection rate

| | | | | Mean GMT |
|---|---|---|---|---|
| | | | | increase |
| Group | Day | Seroconversion^{a} (%) | Seroprotection^{b} (%) | (fold) |
| 1 | 0 | — | 17 (2/12) | — |
| | 14 | 8 (1/12) | 42 (5/12) | 1.4 |
| | 21 | 17 (2/12) | 33 (4/12) | 1.7 |
| | 56 | 33 (4/12) | 58 (7/12) | 2.8^{c} |
| 2 | 0 | — | 33 (4/12) | — |
| | 14 | 17 (2/12) | 50 (6/12) | 1.7 |
| | 21 | 8 (1/12) | 58 (7/12) | 2.1 |
| | 56 | 67 (8/12) | 92 (11/12) | 3.9 |
| 3 | 0 | — | 8 (1/12) | — |
| | 14 | 17 (2/12) | 25 (3/12) | 1.8 |
| | 21 | 33 (4/12) | 67 (8/12) | 3.4 |
| | 56 | 64 (7/11) | 100 (11/11) | 8.1 |

^{a}Seroconversion is defined as either a negative pre-vaccination titer (≦10) to a post-vaccination titer ≧40, or a significant increase in antibody titer, i.e. at least a fourfold increase between pre-and post-vaccination titers where the pre-vaccination titer is ≧10
^{b}Seroprotection rate is defined as the proportion of subjects achieving a post-immunization titer ≧40
^{c}Values meeting CPMP criteria are bold

Although there was a trend toward higher HI titers and higher % seroconversion in a dose-response fashion, there were no statistical differences in GMT, % seroprotection or % seroconversion among the three groups. For all three vaccine dose levels, GMT, % seroconversion and % seroprotection increased as a function of time post-vaccination, with the highest titers on day 56 (see Table 5). In group 1, 33% of subjects had seroconverted by day 56, with 58% achieving seroprotective HAI titers, and a GMP increase of 2.8-fold. In group 2, the seroconversion rate at day 56 was 67%, with 92% of subjects seroprotected and a GMT increase of 3.9-fold. The highest and most consistent titers were observed for group 3 at day 56, where 100% seroprotection was observed, and GMT increased 8.1 fold over baseline. In that group, seroconversion was less than 100%, because some seroprotected subjects did not show a fold increase in titer, due to relatively high baseline HAI titers.

### Discussion

The work described in the present Example provides the first successful generation of an anti-influenza antibody response that is predicted to be effective for influenza prophylaxis.

Immunological analysis demonstrated that all dose levels produced anti-influenza antibody responses. Comparison against the CPMP (Committee for Proprietary Medical Products) guidelines for licensing of annual flu vaccines showed that the 1 μg dose group achieved the criteria on day 56 on Geometirc Mean Titre (GMT), the 2 μg dose group achieved the criteria on day 56 on all three criteria (seroconversion, seroprotectio and GMT) and the 4 μg dose group achieved the criteria on day 21 through GMT and on day 56 on all three criteria.

A total of 320 treatment-emergent adverse events (AEs), including the vaccine site reactions, were reported by 34 (94%) of the 36 subjects dosed. There was 1 SAE reported during the study—a foot fracture—considered unlikely to be related to study vaccine. Seven (7) subjects experienced FDA-reportable AEs that were all considered unlikely to be related to study vaccine. The majority (71%) of the AEs were mild in severity. The most common AE reported was headache (53% of all subjects). No subjects discontinued the study due to an AE. A total of 89 vaccination site-related AEs were reported by 27 of the 36 subjects dosed with mild application site pain being the most common local AE, reported by 12 subjects (33%). No measures of local reactogenicity were assessed as Grade 3 (severe) and therefore considered adverse events. Typical vaccination site reactions include redness/erythema, edema, flaking, discoloration and crust/scab formation.

In conclusion administration of pPJV1671 was well tolerated in all subjects, and elicited potent anti-influenza antibody responses. The top dose of 4 μg met the criteria at 21 days laid down by the CPMP for licensing of annual flu vaccines.

### Example 18

### Non-Clinical Evaluation of Trivalent DNA Vaccine in Pigs

Three DNA vaccine vectors encoding the HA molecules of the three 2001-2002 influenza vaccine strains were constructed to evaluate the immunogenicity of a candidate human trivalent influenza DNA vaccine in a relevant animal model. Historically, the domestic pig has been used as a model for PMED DNA vaccines in humans due to the close similarities in the architectures of human and pig skin. The relevance of the pig model as a predictor of PMED DNA vaccine performance in humans has been demonstrated by a phase I human clinical trial of a hepatitis B surface antigen DNA vaccine in which good vaccine performance in pigs was mirrored in humans.

Samples of three 2001-2002 human influenza vaccine strains (A/Panama/2007/99 (H3N2), A/New Caledonia/20/99 (H1N1), and B/Victoria/5/00) were obtained from the Centers for Disease Control (CDC) and used in reverse transcriptase/polymerase chain reaction (RT-PCR) experiments to generate DNA fragments encoding the corresponding HA antigens. The following steps were employed in developing the final three HA DNA vaccine vectors:
RT-PCR production of dsDNA fragments of RNA segment #4 of A/Panama/2007/99 (H3N2), A/New Calcdonia/20/99 (H1N1), and B/Victoria/5/00.
Propagation of RNA segment #4 DNA clones in a standard pUC19-based vector in E. coli.
Sequence analysis of the HA coding sequence within the RNA segment #4 clones.
A second series of PCR reactions (based on actual HA gene sequence data) to generate DNA fragments from each virus containing the HA coding sequence (without ATG codons) with ends compatible with the pPJV7563 DNA vaccine expression vector (Nhe I and Bsp 120I).
Insertion of the three HA coding sequence fragments into the clinical DNA vaccine vector pPJV7563 yielding the final three DNA vaccine vectors.
Sequence analysis of HA coding sequences in all three vectors confirming that no mutations had occurred.

The resultant vectors employ chimeric promoters of the invention. Thus, the vectors employ the human cytomegalovirus (hCMV) immediate early promoter and 5′ noncoding sequences from immediate early exons 1 and 2. This promoter is linked to the rat insulin intron A and the 5′ untranslated region (UTR) of the hepatitis B virus (HBV) pre-S2 gene. Translation of the HA coding sequence begins at an ATG codon that is fixed in the vector in the context of a consensus Kozak translation initiation sequence. The HA coding sequence is followed by a transcriptional enhancer of HBV (HBVenh). Finally, transcription termination is facilitated by a rabbit β-globin polyadenylation site.

As the natural ATG translation initiation codons of HA genes do not conform to the Kozak consensus sequence for translation initiation, the ATG element supplied by the pPJV7563 DNA vaccine vector was used for translation initiation. Antigen expression from DNA vaccine vectors is generally enhanced using ATG codons that conform to the Kozak consensus. Use of the vector-supplied ATG codon (via insertion at the Nhe I site) results in a minor two-amino acid insertion at the amino terminus of the coding sequence of the HA antigen.

The immunogenicity of these vectors was evaluated in a trivalent formulation in the domestic pig model. The three vectors were mixed 1:1:1 and formulated onto gold particles at a rate of 2 μg total DNA/mg gold, using 0.5 mg gold per administration. Each immunization consisted of two tandem PMED administrations totaling 2 μg DNA and 1 mg of gold. The vaccination regimen involved two such immunizations spaced four weeks apart. Influenza-naïve pigs were divided into two immunization groups based on the actual delivery device employed. One group of animals received trivalent influenza DNA vaccinations using the reusable “research” PMED device that has been successfully used to induce immune responses to numerous antigens in a variety of animals. The second group of animals received immunizations using the clinical device that was used to successfully vaccinate humans using an HBV DNA vaccine. This latter device differs from the research device in that it employs a “single shot” plastic disposable nozzle in place of the 12-shot reusable nozzle associated with the research device.

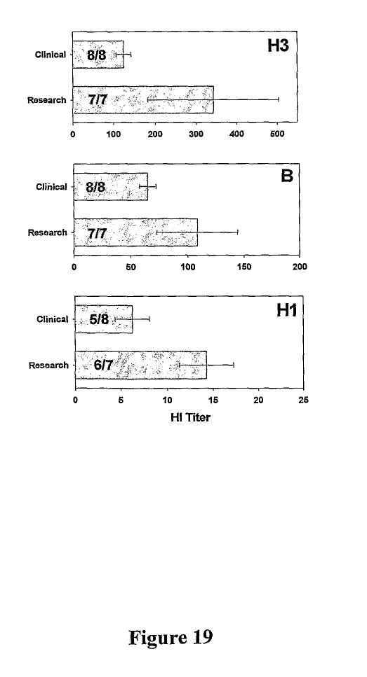

Two weeks following the second immunization, serum samples were collected and hemagglutination-inhibiting (HI) antibody titers specific for the homologous H3, H1 and B viruses were measured. These data are shown in FIG. 19. 100% seroconversion was observed to the H3 and B antigens using both devices with geometric mean HI titers well in excess of the 1:40 surrogate level commonly believed to be required for immunogenicity to influenza. The data obtained indicates that this technology platform will be able to elicit significant responses in humans.

H1-specific immune responses were lower. This was later determined to be due to a rearrangement in the H1 HA gene that occurred in the H1 DNA vaccine vector during plasmid production. This rearrangement went undetected in the initial analysis of the DNA prior to formulation and vaccination, but would be detected by a more stringent analysis. Based on results using the H3 and B vectors, it is likely that H1-specific responses would have been markedly greater had a functional plasmid been utilized.

### Example 19

### Construction of Plasmid pPJV2012

The plasmid pPJV2012 was constructed. pPJV2012 expresses the A and B subunits of the heat labile toxin (LT) of enterotoxigenic Escherichia coli. Expression of functional LT toxin from pPJV2012 in vivo may be used to enhance the immune response to other proteins expressed from plasmids co-administered with pPJV2012.

LT is an 84 kD multimeric protein composed of a single A subunit and a pentamer of identical B subunits. LT is expressed and secreted from E. coli and binds to GM1 ganglioside on intestinal cells via the pentamer of B subunits. The toxin is internalised and the A subunit then activates production of excessive cAMP and disruption of electrolyte balance across the intestinal lumen (Tauschek, et al (2002) Proc Natl Acad Sci, USA 99: 7066-7071). Thus for biological activity of LT expressed in vivo the production of both A and B subunits are required together with signals to mediate secretion from expressing cells. Plasmid vectors encoding the LT A and B subunits have been shown to augment the immune response induced to several viral antigens when co-delivered using particular mediated epidermal delivery (Arrington et al (2002) J Virol 76 (9): 4536-46).

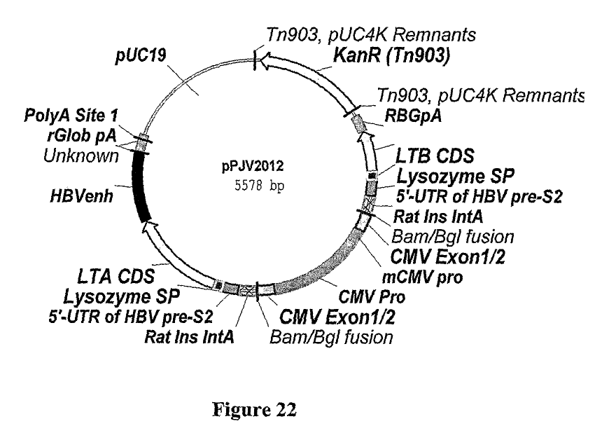

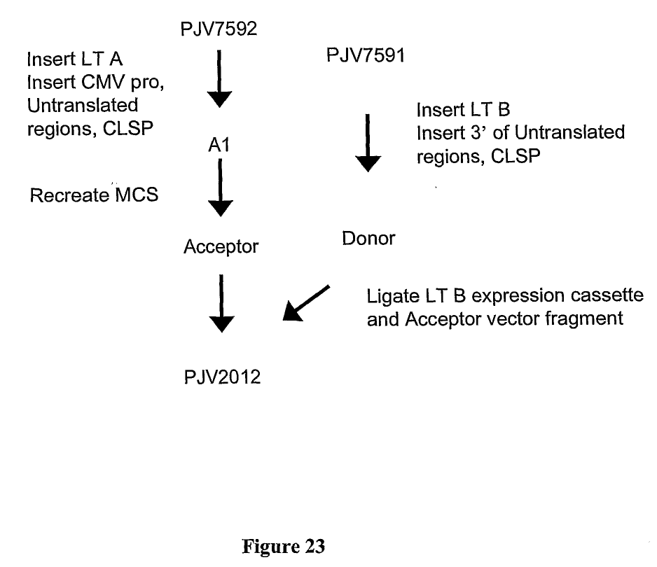

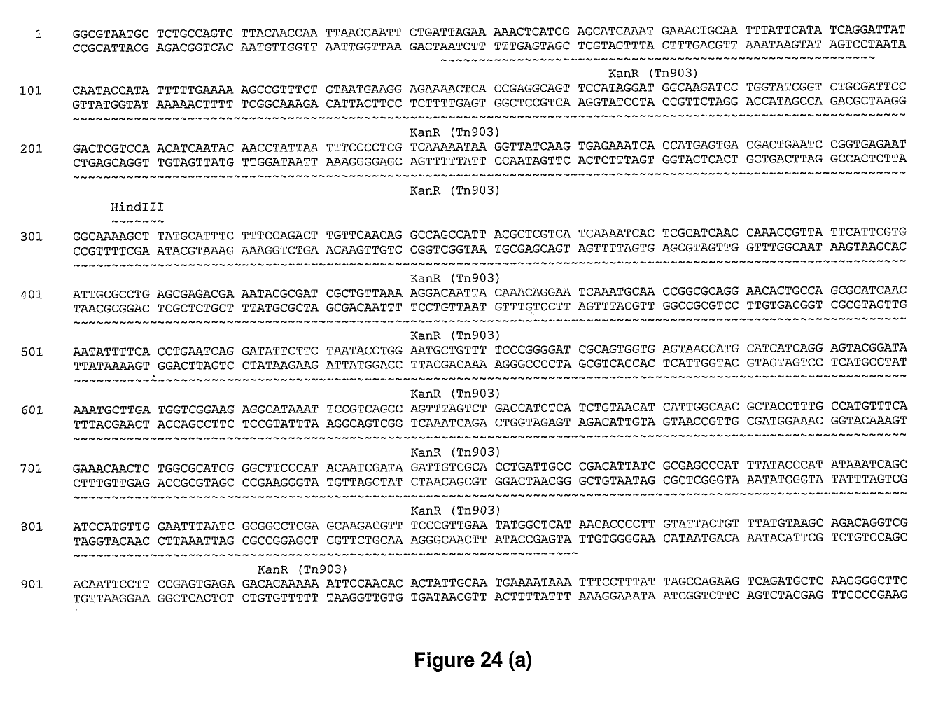

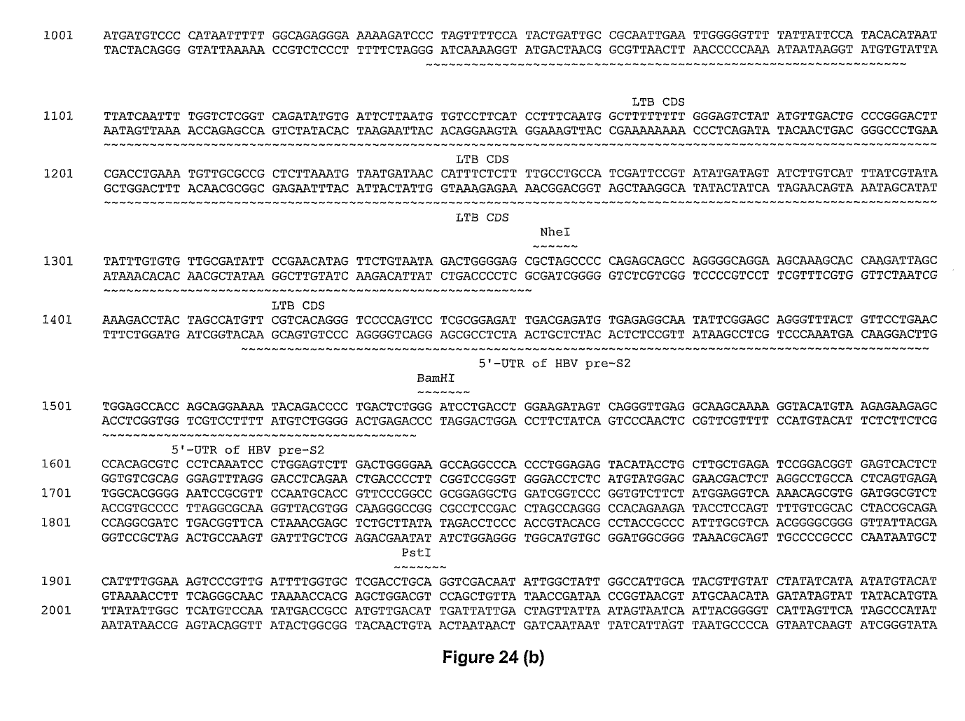

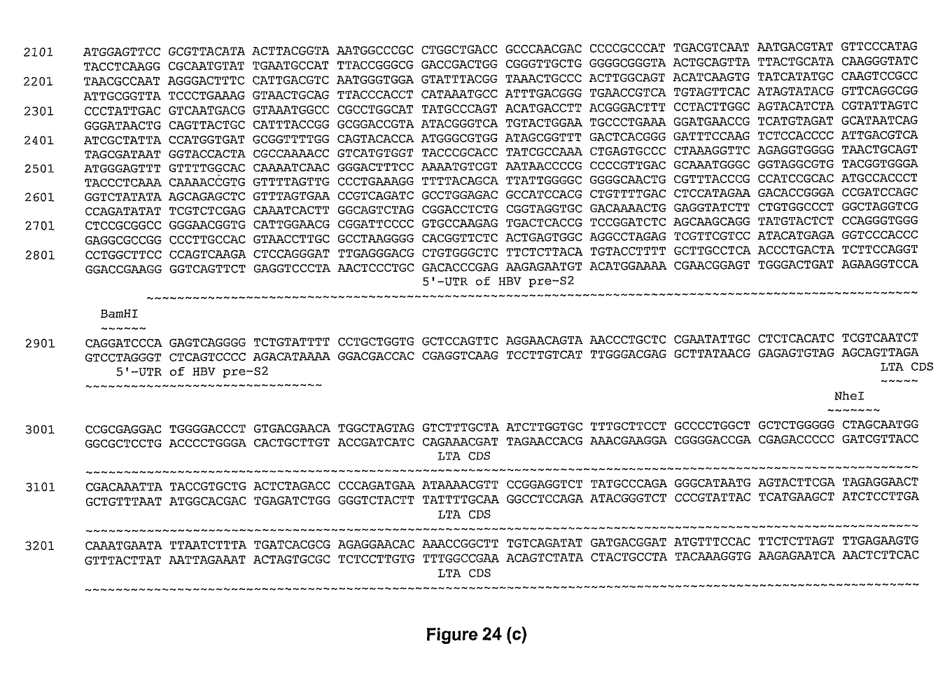

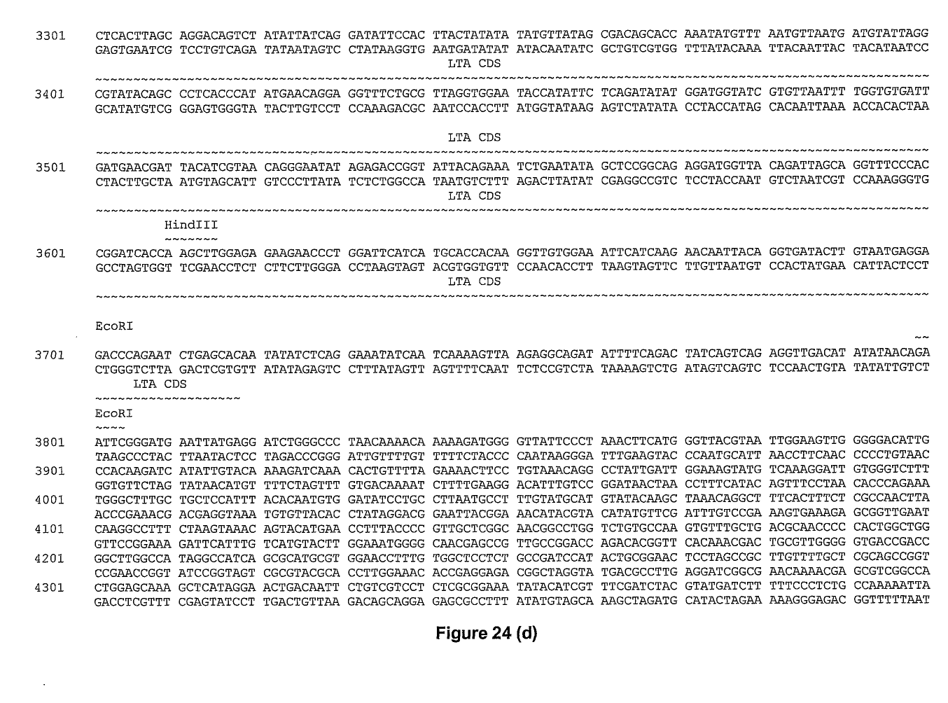

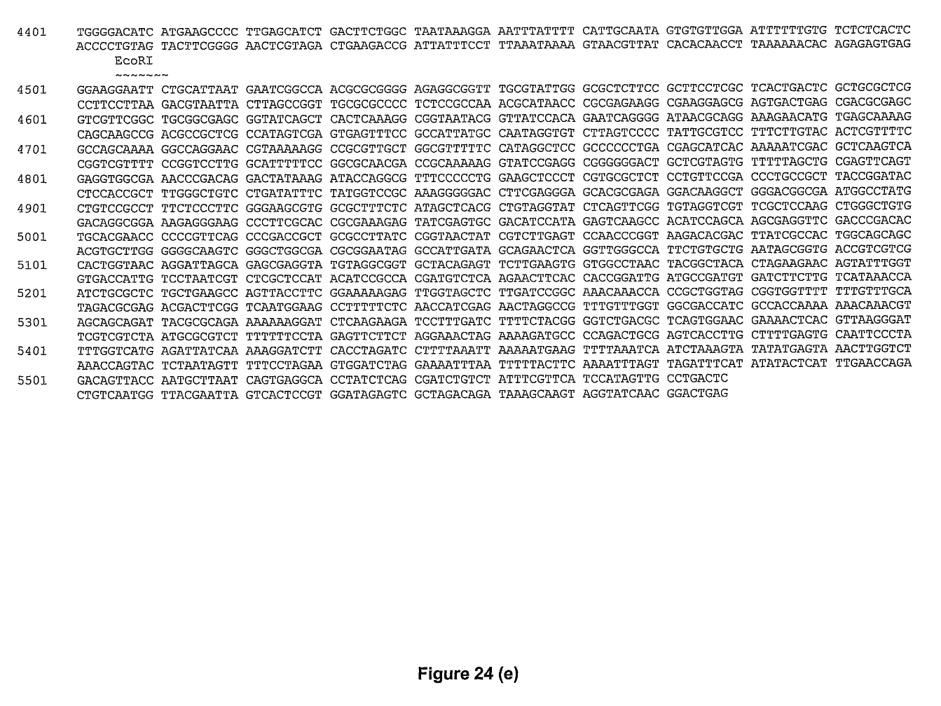

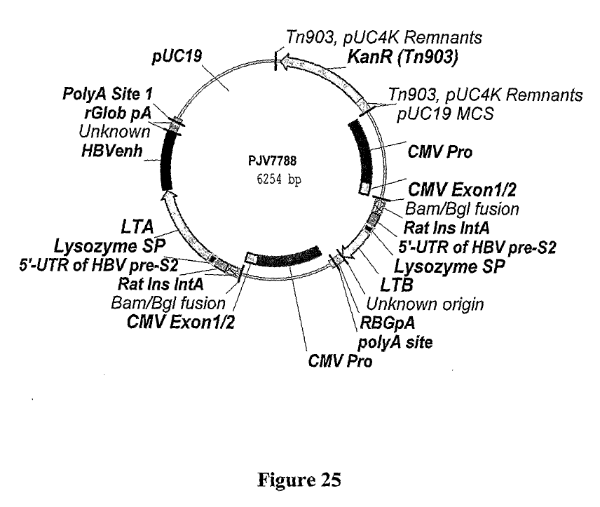

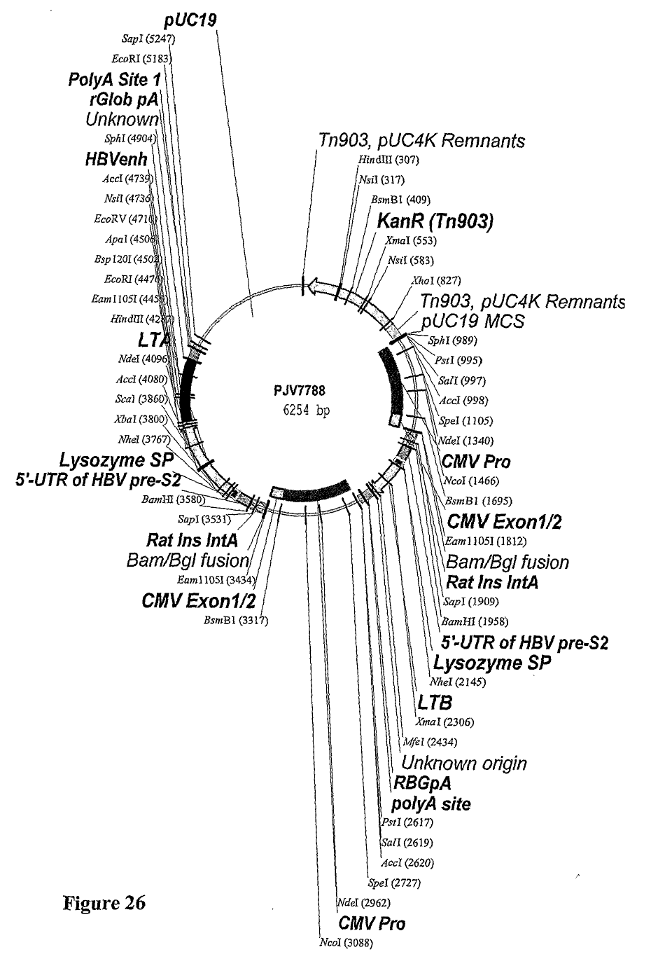

In addition to the coding sequences for the A and B subunits of LT toxin, pPJV2012 also incorporates the chimeric promoter sequences of the invention to ensure effective gene expression, a rabbit beta globin poly A sequence, signal sequences to mediate secretion from expressing cells, a kanamycin resistance gene and a bacterial origin of replication. The plasmid is shown in FIG. 22.

The plasmid pPJV2012 was constructed via the following steps:
PCR amplification of LT A coding sequence from E. coli genomic DNA and insertion into an intermediate plasmid;
Excision of the LT A coding sequence and insertion into an “Acceptor” plasmid under the control of the CMV promoter.
PCR amplification of the LT B coding sequence from E. coli genomic DNA and insertion into an intermediate plasmid under the control of a truncated CMV promoter.
Excision the LT A expression cassette.
   The resultant plasmid, pPJV2012, expression both subunits of LT in mammalian cells.
   Promoter and Enhancer Sequences in pPJV2012

The LT A subunit is expressed from a CMV Immediate Early (1E) promoter. The LT B subunit is expressed from a truncated CMV IE promoter. Additional sequences are included for both genes to improve expression, specifically the HBV pre-S2 5′ UTR (Moriarty et al (1981) Proc Natl Acad Sci USA 78: 2606-2610), CMV exon ½ (consisting of the first two CMV IE exons spliced together by deletion of the natural intron), rat insulin intron A (Lomedico et al (1979) Cell 2: 545-558) and rabbit beta globin poly A (rGpA). To ensure secretion from expressing cells the chicken lysozyme signal peptide (CLSP; Genbank accession number CR390743) has been inserted for both LT subunits. In addition, the HBV env enhancer (Vannice and Levinson (1988) J. Virol. 62: 1305-1313) was inserted after the A subunit to improve expression.

Cloning of the LT A and B subunits

E. coli strain E078:H11 was obtained from ATCC (catalogue #35401), and the A and B subunits were amplified by separate PCR reactions using primers homologous to the 5′ and 3′ ends, designed by reference to the GenBank accession file AB011677. The coding sequences generated for both subunits do not include the sequences encoding the bacterial signal peptides found at the amino termini. The A and B subunit fragments were separately ligated into plasmid WRG7054.

Construction of pPJV2012

The LT A coding sequence was excised and ligated with the vector backbone of pPJV7592 and a fragment derived from pPJV7572 that included the CMV promoter, 5′-untranslated regions and CLSP. The resulting plasmid was designated A1. A1 was cut and ligated with a fragment from pPJV7592 to recreate a multiple cloning site, and the resulting plasmid was designated Acceptor; this provided the LT A component of pPJV2012.

The LT B coding sequence was excised and ligated with the vector backbone of pPJV7591 and to a fragment derived from pPJV7572 that contained the 3′ end of the untranslated regions and the CLSP. The resulting plasmid was designated Donor and provided the LT B component of pPJV2012.

To generate pPJV2012 a fragment from Donor containing the LT B expression cassette was ligated with a vector fragment derived from Acceptor. The resulting plasmid expresses both LT A and B in mammalian cells.

Origin of Nucleotide Sequences in pPJV2012

pPJV2012 has been fully sequence and aligned with databases to assign the origin of each sequence. The derived sequences, shown in Table 6 below, were as expected.

TABLE 6
Identification of Components Comprising Plasmid pPJV2012

| Base Numbers | Component Identification |
|---|---|
| 1-905 | pUC4K-derived sequences including kanamycin |
| | resistance gene |
| 906-1038 | Rabbit beta globin poly A |
| 1039-1351 | LT B subunit coding sequence |
| 1352-1357 | Linker sequence |
| 1358-1417 | Chicken lysozyme signal sequence |
| 1418-1538 | 5′ UTR of HBV pre-S2 |
| 1539-1544 | Linker sequence |
| 1545-1677 | Rat intron A |
| 1678-1683 | Linker sequence |
| 1684-1814 | CMV exon 1/2 |
| 1815-1935 | Truncated CMV IE promoter |
| 1936-1947 | Linker sequence |
| 1948-2632 | CMV promoter |
| 2633-2763 | CMV exon 1/2 |
| 2764-2769 | Linker sequence |
| 2770-2902 | Rat insulin intron A |
| 2903-2908 | Linker sequence |
| 2909-3029 | 5′ UTR of HBV pre-S2 |
| 3030-3089 | Chicken lysozyme signal peptide |
| 3090-3095 | Linker sequence |
| 3096-3818 | LT A coding sequence |
| 3819-3830 | Linker sequence |
| 3831-4363 | HBV env enhancer sequence |
| 4364-4374 | Unknown sequence |
| 4375-4050 | Rabbit beta globin poly A |
| 4051-5578 | pUC19-derived sequence |

In addition a BLAST search was performed for sequence homologies to the entire human genome, looking for sequence similarities in stretches as short as 19 bases. There were “no significant similarities found”; the greatest degree of homology was with a sequence of 68 bases of rGpA, although the longest stretch of identical sequence was only 18 bases.

Comparison of pPJV2012 Sequence to pPJV1671

The sequence of pRJV2012 has been compared to that of pPJV1671. The results are shown in Table 7 below.

TABLE 7
Comparison of the sequence of pPJV2012 with pPJV1671

| | Sequence also present in |
|---|---|
| Base numbers of | pPJV1671 influenza DNA |
| sequence in pPJV2012 | vaccine? |
| 1-896 | Yes |
| 897-905 | No |
| 906-1038 | Yes |
| 1039-1351 | No |
| 1352-1357 | Yes |
| 1358-1417 | No |
| 1418-1538 | Yes |
| 1539-1544 | Yes |
| 1545-1677 | Yes |
| 1678-1683 | Yes |
| 1684-1814 | Yes |
| 1815-1935 | Yes |
| 1936-1947 | Yes |
| 1948-2632 | Yes |
| 2633-2763 | Yes |
| 2764-2769 | Yes |
| 2770-2902 | Yes |
| 2903-2908 | Yes |
| 2909-3029 | Yes |
| 3030-3089 | No |
| 3090-3095 | Yes |
| 3096-3818 | No |
| 3819-3824 | Yes |
| 3825-3830 | Yes |
| 3831-4363 | Yes |
| 4364-4374 | Yes |
| 4375-4505 | Yes |
| 4512-5578 | Yes |

Analysis demonstrates that 4418 of the 5578 base pairs (79%) in pPJV2012 are also present in the pPJV1671 influenza DNA vaccine. Exclusive of the coding sequences for the LT A and B subunits, 4418 of the 4544 base pairs (97%) of pPJV2012 are also present in pPJV1671.

Note that pPJV1671 has undergone biodistribution/integration studies and in each case no evidence of integration was found.

### Example 20

### Construction of the Plasmid pPJV7788 a Plasmid which Expresses the E. coli LT Subunits in Mammalian Cells

The plasmid pPJV7788 was generated using the following plasmids:

pPJV7563 and pPJV7592 which are plasmids derived from pPJV7275, pPJV7284, pPJV7389, pPJV7586 and pPJV7293.

pPJV7590 which is a derivative of pPJV7389 that includes a multiple cloning site (MCS) immediately upstream of the CMV promoter.

pPJV7572 which is a derivative of pPJV7389 that contains the coding sequence for the chicken lysozyme signal peptide (CLSP) directly upstream of the encoded antigen. This allows for extracellular secretion of c-terminally fused antigens.

(i) Construction of pPJV7785 (LTA Acceptor Plasmid)

pPJV7563 was cut with Sal1, blunt ended, and cut with Nde1 to create a vector fragment. pPJV7590 was cut with Sph1, blunt ended, and cut with Nde1 to create an insert fragment containing a MCS and the five-prime end of the CMV promoter. These fragments were ligated to generate pPJV7592.

pPJV7592 was cut with Nco1 and Bgl2 to create a vector fragment. pPJV7572 was cut with Nco1 and Nhe1 to create an insert fragment containing the 3′-end of the CMV promoter, 5′-untranslated regions, and the CLSP. pPJV2004 was cut with Nhe1 and BamH1 to create an insert fragment containing the LTA subunit. These fragments were ligated to generate the plasmid pPJV7785.

(ii) Construction of pPJV7787 (LTB Donor Plasmid)

pPJV7389 was cut with Nco1 and EcoR1 to create a vector fragment. pPJV7572 was cut with Nco11 and Nhe1 to create an insert fragment containing the 3′-end of the CMV promoter, 5′-untranslated regions, and the CLSP. pPJV2005 was cut with Nhe1 and BamH1 to create an insert fragment containing the LTB subunit. pPJV7586 was cut with Bgl2 and EcoR1 to create an insert fragment containing the rabbit beta-globin polyadenylation region. These fragments were ligated to generate pPJV7787.

(iii) Construction of pPJV7788

pPJV7785 was cut with Xho1 and Mfe1 to create a vector fragment containing the LTA expression cassette. pPJV7787 was cut with Xho1 and EcoR1 to create an insert fragment containing the LTB expression cassette. These fragments were ligated to generate pPJV7788 that will express the LTA and LTB subunits when introduced into mammalian cells.

Table 8 below provides the location of the various elements in the construct pPJV7788.

TABLE 8
Component ID for pPJV7788

| Base Numbers | Component |
|---|---|
| 1-44 | Tn903, pUC4K Remnants |
| 45-860 | KanR (Tn903) |
| 861-983 | Tn903, pUC4K Remnants |
| 984-1001 | pUC19 MCS |
| 1002-1686 | CMV Pro |
| 1687-1817 | CMV Exon1/2 |
| 1818-1823 | Bam/Bgl fusion |
| 1824-1956 | Rat Insulin Intron A |
| 1857-1962 | BamH1 site |
| 1963-2083 | 5′-UTR of HBV pre-S2 |
| 2084-2143 | Lysozyme SP |
| 2144-2149 | Nhe1 Site |
| 2150-2462 | LTB |
| 2463-2593 | RBGpA |
| 2594-2623 | Multiple Cloning Site |
| 2624-3308 | CMV Pro |
| 3309-3439 | CMV Exon1/2 |
| 3440-3445 | Bam/Bgl fusion |
| 3446-3578 | Rat Ins IntA |
| 3579-3584 | BamH1 |
| 3585-3705 | 5′-UTR of HBV pre-S2 |
| 3706-3765 | Lysozyme SP |
| 3766-3671 | Nhe1 site |
| 3772-4494 | LTA |
| 4495-4506 | Polylinker |
| 4507-5039 | HBV enh |
| 5040-5050 | Unknown origin. |
| 5051-5181 | rGlob pA |
| 5182-5187 | EcoR1 site |
| 5188-6254 | pUC19 |

### Example 21

### Construction of pPML7789, a Plasmid that Expresses the H5/VN1194 Hemagglutinin Protein in Mammalian Cells

A further construct, pPML7789, capable of expressing the H5/VN1194 Hemagglutinin Protein utilizing a chimeric promoter of the invention was generated. The construct pPJV7563 described in Example 5 was used as the backbone for the construct pPML7789.

A plasmid harboring the coding sequence for H5/VN1194 HA gene was used as the template for PCR. PCR primers were designed with sites at the five and three-prime ends to allow insertion into pPJV7563. pPJV7563 was cut with Nhe1 and Bsp120I to generate a vector fragment. The PCR fragment amplified from the H5/VN1194 template was cut with the same enzymes to generate an insert fragment. These two fragments were ligated to produce pPML7789. The coding and flanking sequences were verified by sequencing.

The nucleotide positions of the various elements in pPML7789 are indicated in Table 9 below.

TABLE 9
Components of construct pPML7789

| Nucleotide Position | Element |
|---|---|
| Start: 1 End: 44 | Tn903, pUC4K Remnants |
| Start: 45 End: 860 | KanR (Tn903) |
| Start: 861 End: 896 | Tn903, pUC4K Remnants |
| Start: 897 End: 902 | pUC19 MCS |
| Start: 903 End: 1587 | CMV Pro |
| Start: 1588 End: 1718 | CMV Exon1/2 |
| Start: 1719 End: 1724 | Bam/Bgl fusion |
| Start: 1725 End: 1857 | Rat Ins IntA |
| Start: 1858 End: 1863 | BamH1 site |
| Start: 1864 End: 1984 | 5′-UTR of HBV pre-S2 |
| Start: 1985 End: 1993 | ATG-Nhe |
| Start: 1994 End: 3699 | VN1194H5 |
| Start: 3700 End: 3705 | Bsp120I site |
| Start: 3706 End: 4238 | HBV enh |
| Start: 4239 End: 4249 | Unknown linker sequence |
| Start: 4250 End: 4380 | rGlob pA |
| Start: 4341 End: 4341 | PolyA Site |
| Start: 4381 End: 4386 | EcoR1 site |
| Start: 4387 End: 5453 | pUC19 |

### Example 22

### Potent Protective Cellular Immune Responses Generated by DNA Immunisation with HSV-2 ICP27 and E. coli Heat Labile Toxin

### Materials and Methods

Viruses

HSV-2 viruses (MS strain, ATCC VR-540 and HG52 strain G gift from Nancy Sawtell, University of Cincinnati) were grown on VERO cells (ATCC CCL-81) and purified on sucrose gradients prior to use.

DNA Vaccine and DEI Vectors

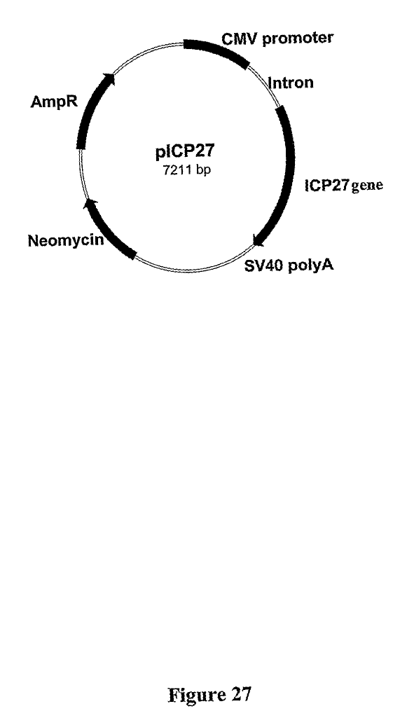

The plasmid encoding HSV-2 ICP27 (FIG. 27) was generated by inserting a PCR fragment incorporating the entire ICP27 gene (UL54) into pTarget (Promega, Madison Wis.). The PCR fragment was amplified from purified genomic DNA of the MS strain of HSV-2 using 5′ (GCCACTCTCTTCCGACAC, SEQ ID NO:63) and 3′ (CAAGAACATCACACGGAAC, SEQ ID NO:64) primers. The amplified sequences correspond to nucleotides 114,523 to 116,179 of the published genome sequence of the HG52 clone of HSV-2 (GenBank accession number NC_{—}001798). The pICP27 was sequenced in its entirety by the DNA Sequencing Core Facility at the University of Wisconsin, Madison.

Plasmid PJV2012 encodes both the A and B subunits of LT from a single vector and is shown in FIG. 22. The construction of pPJV2012 is described in Example 19. In pPJV2012, the LT A subunit gene is expressed from a transcription unit consisting of the following components: the human cytomegalovirus (hCMV) immediate early promoter, fused exons 1 and 2 from hCMV (non-coding), rat insulin intron A, 5′ non-coding region of the hepatitis B virus (HBV) pre-S2 gene, ATG codon, lysozyme signal peptide coding sequence, LT A subunit coding sequence, 3′ non-coding HBV transcriptional enhancer region, and the rabbit beta globin polyadenylation sequence.

pPJV2012 also encodes the LT B subunit product from a second transcription unit running in the opposite direction (FIG. 22). The LT B transcription unit is similar to that encoding the LT A product but does not contain additional copies of the hCMV and HBV transcriptional enhancer elements. The hCMV and HBV enhancer elements located in the LT A transcription unit also serve the LT B transcription unit.

Plasmid PJV2013 (not shown) is similar to pPJV2012 but encodes both the A and B subunit products of cholera toxin from a single vector. The pPJV2013 functional map is identical to that shown for pPJV2012 in FIG. 22.

DNA Vaccination Via Particle-Mediated Epidermal Delivery (PMED)

PMED DNA vaccination of 6 to 8 week old female Balb/c mice was as previously described (Arrington et al, J. Virol. 76, 4536-4546, 2002) except that each immunization consisted of a single PMED delivery to the ventral abdominal skin in which each delivery contained 0.5 mg of gold coated with a total of 0.5 μg of the DNA vaccine/DEI vector formulation. Two such “single shot” immunizations were administered 4 weeks apart and animals were sacrificed or challenged 2 weeks following the second or “booster” immunization.

Peptide Pools and Peptides

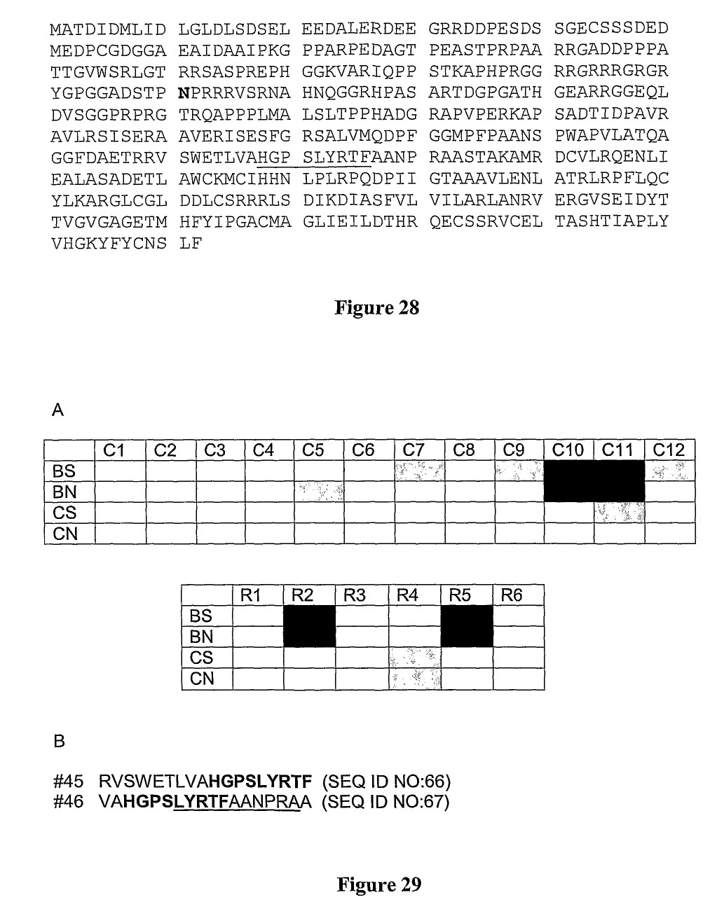

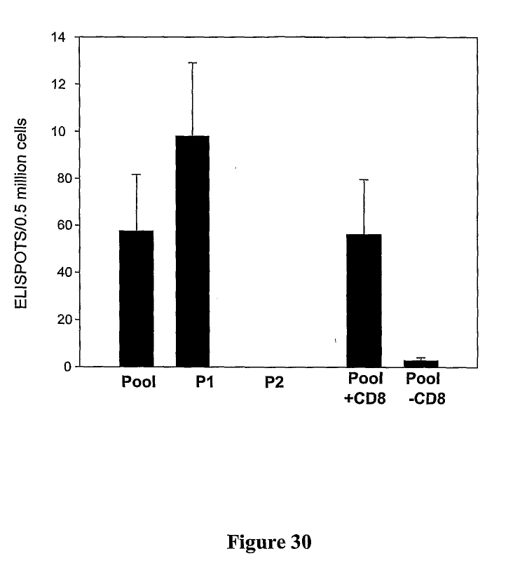

The derived amino acid sequence for ICP27 from the MS strain virus was used as a template for the library. The ICP27 protein is highly conserved between the HG52 and MS sequence (see FIG. 28 and SEQ ID NO:65) thus the library would be capable of detecting responses to both strains. A peptide library was synthesized (Mimotopes, Fisher Scientific) to span the entire sequence of ICP27 using peptides 18 amino acids in length that had an 11 amino acid overlap with the adjacent peptide. A total of 72 peptides were made.

To facilitate the identification of positive peptides the library was divided into peptide pools using the strategy described by Tobery et al J. Immunol. Methods 254, 59-66, 2001. Peptide pools were generated using the pattern shown in Table 10. There were 12 pools (named C1-C12) with 6 peptides per pool and 6 pools (R1-R6) with 12 peptides per pool. Pools containing peptides #45 and #46 are in bold type.

TABLE 10
Peptide Pool Compositions

| | C1 | C2 | C3 | C4 | C5 | C6 | C7 | C8 | C9 | C10 | C11 | C12 |
|---|---|---|---|---|---|---|---|---|---|---|---|---|
| R1 | 1 | 3 | 5 | 7 | 9 | 11 | 13 | 15 | 17 | 19 | 21 | 23 |
| R2 | 25 | 27 | 29 | 31 | 33 | 35 | 37 | 39 | 41 | 43 | 45 | 47 |
| R3 | 49 | 51 | 53 | 55 | 57 | 59 | 61 | 63 | 65 | 67 | 69 | 71 |
| R4 | 4 | 6 | 8 | 10 | 12 | 14 | 16 | 18 | 20 | 22 | 24 | 26 |
| R5 | 28 | 30 | 32 | 34 | 36 | 38 | 40 | 42 | 44 | 46 | 48 | 50 |
| R6 | 52 | 54 | 56 | 58 | 60 | 62 | 64 | 66 | 68 | 70 | 72 | 2 |

ELISPOT Assays

IFN-γ ELISPOT assays were performed on fresh splenocytes and lymph node cells as previously described except the antigens for stimulation were single peptides or peptide pools as defined for each experiment in the Results section. In some instances, prior to the ELISPOT assay, T-cell populations were depleted by magnetic beads (Dynal) following manufacturers instructions.

Cytometric Bead Array (CBA) Assays

Fresh splenocytes were collected two weeks following the second immunization and resuspended at 1×10⁷ cells per ml in SM (RPMI medium+10% fetal bovine serum supplemented with MEM sodium pyruvate, non-essential amino acids, and 2-mercaptoethanol (Invitrogen, Carlsbad, Calif.)). Cell samples were plated at 1×10⁶ cells (100 μl) per well in 96-well flat-bottom plates. The splenocytes were treated with 100 μl aliquots of the ICP27 peptide in SM or medium-alone. The final peptide concentration was 10⁻⁸ M. Control splenocytes from naïve animals were also plated with and without peptide and with and without Con-A (2.5 mg/ml final concentration) to serve as negative and positive controls, respectively.

Plates were incubated at 37° C. for 48 hours and then 160 μl of each sample was collected and stored at −20° C. until assayed using the BD Cytometric Bead Array (CBA) Mouse Th1/Th2 kit (BD Biosciences, San Jose, Calif. (cat. #551287)). Briefly, this kit uses 5 bead populations with distinct fluorescence intensities coated with capture antibodies specific for mouse IL-2, IL-4, IL-5, INF-γ, and TNF-α. The five bead populations are mixed together to form the array that is resolved on the FL3 channel of the BD FACSCalibur flow cytometer. Cytokine capture beads are mixed with the PE-conjugated detection antibodies and then incubated with standards or test samples to form sandwich complexes. BD CBA Analysis Software was used to generate results after sample acquisition. The sample intensity for each cytokine is compared to standard curves to facilitate quantification of cytokine concentrations. Samples were run neat or at a dilution of 1:10 according to the manufacturers instructions.

HSV-2 Virus Challenge

Anesthetized Balb/c mice were intranasally challenged with 30 μl of PBS containing approximately 50LD₅₀ (2×10⁶ PFU) of HSV strain MS. Mice were followed for 20 days after infection and scored for morbidity and mortality. Morbidity was scored on a scale of 0 to 4 according to the following schedule: 4, healthy; 3, ruffled fur, sneezing; 2, sores on eyes or rump, reduced movement; 1, hunched, little movement; 0, dead. Mice that were treated with IFN-γ- (eBioscience, San Diego, Calif. (cat. # 16-7311) and/or TNF-α- (cat. #16-7332) specific monoclonal antibodies around the time of challenge received intraperitoneal injections containing 90 μg of antibody on days −2, 0, 2, 4, 6, and 8 relative to virus challenge. In T-cell depletion experiments, mice received intraperitoneal injections containing 200 μg of antibody specific for either the CD4 or CD8 populations on days −2 and 0 relative to virus challenge (Functional Grade purified anti-mouse CD4, eBioscience Cat#: 16-0041; Functional Grade purified anti-mouse CD8, eBioscience; Cat#: 16-0081).

### Results

Identification of a Strong CD8 Epitope in Balb/c Mice.

To identify T-cell responses against the ICP27 protein, an IFN-γ ELISPOT assay was carried out on cells recovered from Balb/c and C57B1/6 mice infected with an attenuated strain of HSV-2 (HG52 strain G). A peptide library composed of peptides spanning the entire length of the ICP27 coding sequence was used to stimulate the T-cells. The peptides were 18 amino acids in length and overlapped neighboring peptides by 11 amino acids. Peptide pools were composed of either 6 peptides (Pools C1-C12) or 12 peptides (Pools R1-R6) as described in Materials and Methods and Table 10, a configuration that facilitated identification of positive peptides within the library. Spleen and lymph node cells recovered from Balb/c mice 7 days after infection showed very strong responses to peptides within pools C10, C11, R2 and R5 (FIG. 29A) with weaker responses in several other pools. Only weak responses were detected in cells recovered from C57B1/6 mice. The pattern of positive responses was found to be similar in the assayed spleen and lymph node cell populations.

Based on the IFN-γ ELISPOT results, two adjacent peptides (#45, #46) were predicted to contain the dominant T-cell epitope. This was confirmed by testing each peptide individually (data not shown). Peptides 45 and 46 stimulated strong IFN-γ secretion and contained a homologous nine amino acid sequence HGPSLYRTF (FIG. 29B, SEQ ID NO:68) that is predicted to bind the Dd allele. Interestingly, peptide 46 also contains a region that corresponds to an epitope previously described in Balb/c mice for ICP27 from HSV-1 (Banks et al, J. Virol. 67, 613-616, 1993). Comparison of ICP27 sequences in HSV-1 and HSV-2 showed that the region differs by a single amino acid; the alanine (A) residue in the HSV-2 ICP27 (LYRTFAANPRA, SEQ ID NO:69) is replaced by a glycine (G) in HSV-1 (LYRTFAGNPRA, SEQ ID NO:70). The region, however, is not present in peptide 45 even though this peptide stimulates an extremely strong response.

To determine the relative importance of the two potential epitopes in peptide 46 in the IFN-γ ELISPOT assay, the peptides LYRTFAANPRA and the HGPSLYRTF were synthesized and tested. Spleen cells isolated from infected mice responded strongly to the HGPSLYRTF peptide, but no response above background was detected to the LYRTFAANPRA sequence. Using magnetic beads to deplete T-cell populations prior to the IFN-γ ELISPOT assay, resulted in a strong CD8+ ICP27 response. The data indicates that a strong CD8 response is generated in Balb/c mice towards the HSV-2 ICP27 protein that is specific for the sequence HGPSLYRTF.

In Vitro Immune Responses to ICP27 Vaccine

A DNA vaccine, “pICP27”, was generated using ICP27 sequences from the MS strain of HSV-2 (FIG. 27). The sequenced construct had only two nucleotide differences from the published HG52 ICP27 gene sequence (FIG. 28). There was a G to A change at nucleotide 57 which would be silent, and an A to C change at 484 would change a lysine in the HG52 strain to an asparagine in the MS version. Thus, the MS strain ICP27 protein expressed from the DNA vaccine would be almost identical to the HG52 strain version of the protein with no difference in the region of the putative CD8 epitope.

The pICP27 DNA vaccine was used to immunize Balb/c and C57B1/6 mice by PMED. Splenocytes and lymph node cells were tested in the IFN-γ ELISPOT assay against the panel of pools described in FIG. 29. The pattern of positive responses to the ICP27 peptides found in DNA immunized mice were the same as those found in the infected mice (data not shown).

In Vivo Responses to ICP27 Vaccine

The in vivo activity of immune responses generated by pICP27 DNA vaccination was studied in a prophylactic intranasal infection model in Balb/c mice. Infection with various doses of the MS strain of HSV-2 in 12-week-old Balb/c mice established an LD₅₀ of approximately 3×10⁴ PFU (data not shown). In DNA protection experiments, a virus challenge dose of 50 LD₅₀ was used because this dose consistently resulted in 100% mortality in naïve animals.

A series of experiments showed that immunization with pICP27 DNA alone (prime and boost) had limited ability to protect mice. Therefore, to improve protection DEI vectors encoding cholera toxin (CT) or the heat labile toxin from E. coli (LT) were co-delivered with the ICP27 DNA vaccine. The A and B toxin subunits were expressed from the same plasmid for each of the two toxins.

Mice were immunized with the ICP27 DNA vaccine, either alone, or supplemented with CT or LT DEI vectors at a 9:1 ratio (0.45 μg antigen DNA+0.05 μg DEI vector DNA). A total of 16 animals per immunization group were employed, half of which were challenged with virus and the other half sacrificed at the time of challenge for ICP27-specific cytokine production.

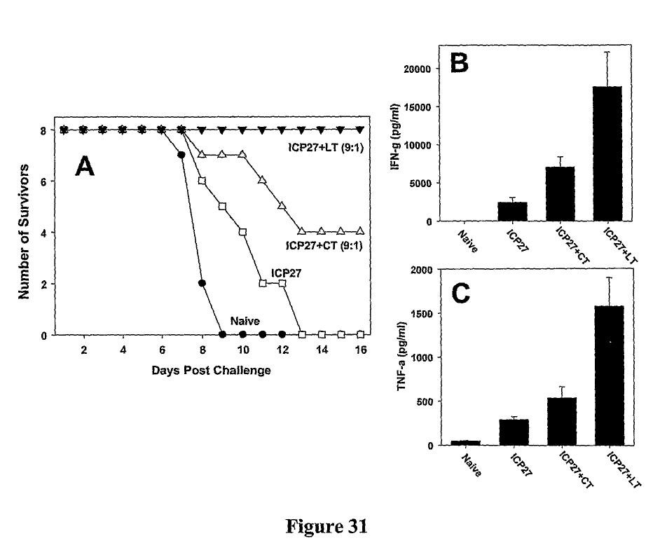

The results of this study are shown in FIG. 31. The results show that immunization with the ICP27+CT and ICP27-only formulations provided partial protection and no protection, respectively, whereas 100% protection was observed for the ICP27+LT formulation (FIG. 31A). These results confirm that co-delivery of DEI vectors with the ICP27 vector enhance protection and reinforce the superiority of LT over CT as an immuno stimulator in DNA immunization. Quantification of cytokine production following in vitro stimulation with the CD8 epitope peptide using the CBA kit demonstrated a good correlation between challenge survival and the levels of both INF-γ (FIG. 31B) and TNF-α production (FIG. 31C).

The Role of Cytokines and T-Cell Populations

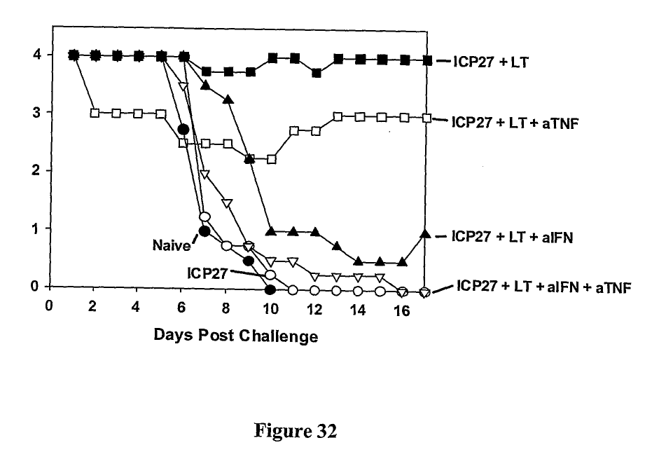

The role of ICP27-specific production of IFN-γ or TNF-α in the protection of challenged animals was evaluated. pICP27+pPJV2012 (LT)-vaccinated animals were treated with INF-γ- and/or TNF-α-specific monoclonal antibodies for several days around the time of challenge to neutralize these cytokines in vivo. Morbidity data is shown in FIG. 32. As before, animals that received two ICP27+LT DEI vaccinations were completely protected from the challenge and exhibited only a mild transient morbidity (fur ruffling in 1 of 4 animals) while 100% of naïve mice or mice immunized with the pICP27 vector alone succumbed to the challenge. In the group immunized with the ICP27+LT DEI formulation and subsequently treated with anti-TNF-α, one death was observed immediately after intranasal challenge inoculation due to complications of anesthesia. Importantly, the three remaining mice showed only transient fur ruffling and fully recovered indicating that INF-α production did not likely contribute significantly to protection. In contrast, the two groups of ICP27+LT DEI-immunized mice that received anti-IFN-γ or anti-IFN-γ+anti-TNF-α at the time of challenge became intensely morbid and 7 of 8 animals died, clearly demonstrating the importance of IFN-γ as an essential mediator of protection.

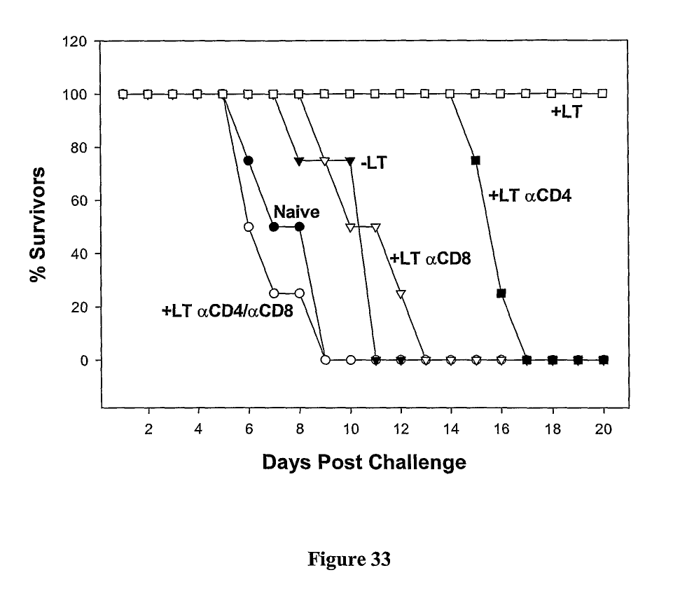

To examine the role of the CD4 and CD8 populations in protection, antibodies to CD4 or CD8 were used to deplete these populations prior to infectious challenge (FIG. 33). When mice were followed for mortality over 20 days an interesting pattern emerged. As expected, mice given pICP27 with empty vector were not significantly protected from infection. pICP27+LT DEI vaccination provided 100% survival, whereas, removal of both CD4 and CD8 cells from similarly immunized mice abrogated the protective effects of vaccination and made the mice susceptible to about the same level as naïve mice. In mice immunized with the pICP27+LT DEI vaccine, depletion of the CD8 population prior to infection caused mice to succumb to infection indicating an important role of these cells in protection from infection or encephalitis. In the pICP27+LT DEI vaccinated mice, depletion of the CD4 population introduced a nine day delay (relative to naïve mice) before these mice also became ill and died, suggesting a role for CD4 T-cells for the long term survival.

### Example 23

### Immunogenicity of Influenza H5N1 DNA Vaccine in Mice

### Materials and Methods

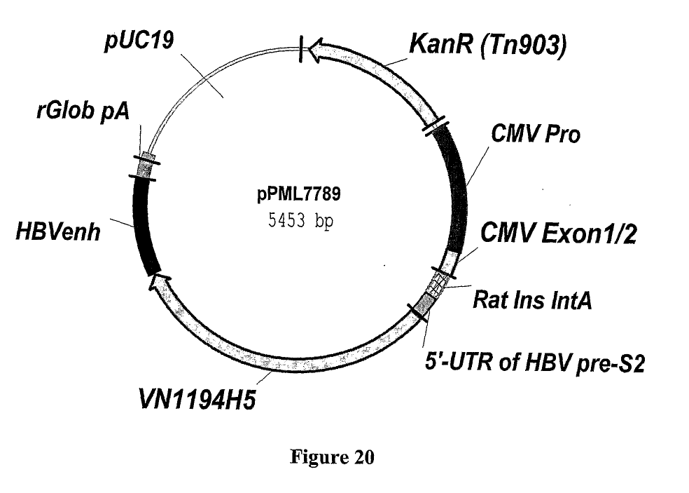

DNA plasmid pPML7789 (Example 21, FIG. 20) encoding the HA of Influenza A/Vietnam/1194/2004 [H5N1] virus was precipitated onto gold particles and delivered to mice by particle mediated epidermal delivery (PMED) using PowderMed's proprietary delivery technology. Delivery was effected with or without, as an adjuvant, an additional plasmid pPJV2012 (Example 19, FIG. 22) encoding E. coli heat labile toxin subunits A and 13. When pPJV2012 is present, this additional plasmid and the pPML7789 plasmid were precipitated onto the same gold particles. That was achieved by first premixing pPJV2012 and pPML7789 in liquid form at weight ratio of 1:9 and then co-precipitating the plasmids onto a population of gold particles. As a negative control, gold particles on which no plasmid has been precipitated were delivered by PMED. The mice were as follows:

Strain: Balb/C mice

Number: 36

Gender: Female

Weight Range: 15.0-19.6 g (on the day of the first procedure)

Age: 42-49 days (on the day of arrival)

Diet: RM1 pelleted

Supplier: Charles River UK Ltd

The mice were divided into six groups as shown in Table 11 below. The procedures performed on the mice and the days on which the procedures took place are shown in Table 12 below. An HAI assay was carried out on the sera according to standard procedures.

TABLE 11
Treatment Group Assignment for Animals

| Group | Route of | # of animals in | |
|---|---|---|---|
| # | Admin | each group | Treatment |
| 1 | Epidermally | 6 | Negative control |
| | | | 1 dose |
| | | | Bleed Day −1, bleed and cull |
| | | | Day 14 |
| 2 | Epidermally | 6 | Negative control |
| | | | 2 doses |
| | | | Bleed Day −1 and 21, bleed and |
| | | | cull Day 36 |
| 3 | Epidermally | 6 | pPML7789 |
| | | | 1 dose |
| | | | Bleed Day −1, bleed and cull |
| | | | Day 14 |
| 4 | Epidermally | 6 | pPML7789 |
| | | | 2 doses |
| | | | Bleed Day −1 and 21, bleed and |
| | | | cull Day 36 |
| 5 | Epidermally | 6 | pPML7789 + pPJV2012 |
| | | | 1 dose |
| | | | Bleed Day −1, bleed and cull |
| | | | Day 14 |
| 6 | Epidermally | 6 | pPML7789 + pPJV2012 |
| | | | 2 doses |
| | | | Bleed Day −1 and 21, bleed and |
| | | | cull Day 36 |

TABLE 12
The Following Procedures were Performed on the Days Marked X

| Day of Study | 0 | 14 | 21 | 35 |
|---|---|---|---|---|
| Transponder implantation | X | | | |
| Body weight | X | X | X^{b} | X^{b} |
| Vaccination | X | | X^{b} | |
| Health Score | X | X | X^{b} | X^{b} |
| Serum for antibody | X | X^{a} | X^{b} | X^{b} |
| Harvest spleens | | X^{a} | | x^{b} |
| Culling | | X^{a} | | x^{b} |

^{a}Groups 1, 3 & 5 only
^{b}Groups 2, 4 & 6 only

### Results

HAI Titres Against NIBRG-14 [H5N1] Virus were Determinal

All controls were observed to be within acceptable limits. The negative control article (NIBRG-14 [H5N1] virus) was negative on all plates. The positive control article (turkey red blood cells) was positive on all plates. The second positive control article (serum containing antibodies to Influenza A/Chicken/Scotland/59 [H5N1] virus) ran on all plates to a geometric mean of 28, 40, 56 or 80HAIU (within acceptable limits of 40 HAIU+/−2-fold).

All samples on days 0, 14 and 21 were <10HAIU (below the limit of detection) with the exception of ID:1074 (group 6) on Day 21 which could not be bled, and ID:1059 (group 4) on Day 21 for which there was insufficient serum obtained to perform the assay.

For the Day 35 bleeds, with the exception of ID:1074 (group 6) which could not be bled, the geometric mean titre of each sample was calculated, along with the group arithmetic mean, median and standard deviation for each group. These are shown in Table 13 below.

TABLE 13
HAI Titres for Groups 2, 4 & 6 of Serum obtained on Day 35

| | Sample | GEO mean | Arithmetic | | |
|---|---|---|---|---|---|
| Group | No. | titre | mean | Median | SD |
| 2 | 1047 | <10 | <10 | <10 | 0 |
| | 1048 | <10 | | | |
| | 1049 | <10 | | | |
| | 1050 | <10 | | | |
| | 1051 | <10 | | | |
| | 1052 | <10 | | | |
| 4 | 1059 | 28 | 28 | 24 | 10 |
| | 1060 | 40 | | | |
| | 1061 | 40 | | | |
| | 1062 | 20 | | | |
| | 1063 | 20 | | | |
| | 1064 | 20 | | | |
| 6 | 1071 | 160 | 164 | 160 | 40 |
| | 1072 | 226 | | | |
| | 1073 | 160 | | | |
| | 1074 | * | | | |
| | 1075 | 160 | | | |
| | 1076 | 113 | | | |

* Data unavailable due to the premature death of one animal

The results show that the pPML7789 plasmid is immunogenic in mice and that this immunogenicity is enhanced by the inclusion of pPJV2012.

Accordingly, novel nucleic acid constructs, compositions comprising various constructs, and nucleic acid immunization techniques using these constructs have been described. Although preferred embodiments of the subject invention have been described in some detail, it is understood that variations can be made without departing from the spirit and the scope of the invention.

1-114. (canceled)

115. A nucleic acid construct comprising a chimeric promoter sequence and a coding sequence in operable linkage with the chimeric promoter, where the coding sequence encodes an influenza virus antigen, an immunogenic fragment thereof or an immunogenic variant of said antigen or fragment having at least 80% amino acid homology to said antigen or fragment and the chimeric promoter sequence comprises:
(a) a hCMV immediate early promoter sequence;
(b) exon 1 and at least a part of exon 2 of the hCMV major immediate early gene; and
(c) a heterologous intron provided in place of the intron A region of the hCMV major immediate early gene.

116. A nucleic acid construct according to claim 115, wherein the encoded antigen is influenza hemaglutinin (HA), influenza neuraminidase (NA), M2, an immunogenic fragment of either or an immunogenic variant having at least 80% amino acid sequence homology to any of said NA, M2 or fragment.

117. A nucleic acid construct suitable for delivery to a subject for inducing an immune response against influenza virus hemagglutinin (HA) antigen, which construct comprises:
a chimeric promoter sequence comprising:
(a) a hCMV immediate early promoter sequence;
(b) exon 1 and at least a part of exon 2 of the hCMV major immediate early gene; and
(c) a heterologous intron provided in place of the intron A region of the hCMV major immediate early gene; and/or
(ii) a coding sequence in operable linkage with the chimeric promoter, where the coding sequence encodes one or more an influenza virus hemagglutinin (HA) antigen, an immunogenic fragment thereof or an immunogenic variant of said antigen or fragment having at least 80% amino acid homology to said antigen or fragment; and/or
(iii) a non-translated leader sequence which is derived from HBVpreS2 antigen sequence, HBV e-antigen sequence or HSV type 2gD antigen sequence and which is in operable linkage with the chimeric promoter; and/or
(iv) an enhancer sequence which is derived from a 3′ untranslated region (UTR) of a HBsAg sequence or of a simian CMV immediate early gene sequence, which is in operable linkage with the chimeric promoter and which is downstream of coding sequence.

118. A nucleic acid construct according to claim 115, wherein the construct encodes more than one influenza antigen, immunogenic fragment or immunogenic variant.

119. A nucleic acid construct comprising a chimeric promoter according to claim 115 further comprising a coding sequence in operable linkage with the chimeric promoter, where the coding sequence encodes an ADP ribosylating bacterial toxin subunit, a fragment thereof with adjuvant activity or a variant of either thereof having adjuvant activity and having at least 80% amino acid homology with said subunit or fragment and the chimeric promoter sequence comprises.

120. A nucleic acid construct according to claim 119, wherein the ADP ribosylating bacterial toxin subunit is selected from cholera toxin subunit A, cholera toxin subunit B, E. coli heat labile toxin subunit A and E. coli heat labile subunit B.

121. A nucleic acid construct according to claim 119, wherein the nucleic acid construct comprises two coding sequences which each comprise a different said subunit, fragment or variant and each of which is operably linked to a said chimeric promoter.

122. A nucleic acid construct according to claim 121, wherein the two coding sequences encode cholera toxin subunit A and cholera toxin subunit B respectively or E. coli heat labile toxin subunit A and E. coli heat labile subunit B respectively.

123. A nucleic acid construct according to claim 121, wherein the said two coding sequences are in inverse orientation or the same orientation.

124. A nucleic acid construct according to claim 115, wherein the hCMV immediate early promoter sequence of (a) comprises:
(i) the nucleotide sequence of SEQ ID NO:1, nucleotides 903 to 1587 of SEQ ID NO:54, nucleotides 1815 to 1935 of SEQ ID NO:61, nucleotides 1948 to 2632 of SEQ ID No: 61, nucleotides 1002 to 1686 of SEQ ID No: 62 and/or nucleotides 2624 to 3308 of SEQ ID No:62;
(ii) a functional variant of (i) which has at least 80% nucleotide sequence homology to one or more of the sequences of (i); or
(iii) a functional fragment of (i) or (ii).

125. A nucleic acid construct according to claim 115, wherein the exon of (b) comprises:
(i) the nucleotide sequence of SEQ ID No. 2, the nucleotide sequence of nucleotides 1588 to 1718 of SEQ ID No. 54, nucleotides 1684 to 1814 of SEQ ID No: 61, nucleotides 2633 to 2763 of SEQ ID NO:61, nucleotides 1687 to 1817 of SEQ ID No: 62 and/or nucleotides 3309 to 3439 of SEQ ID No: 62;
(ii) a functional variant of (i) which has at least 80% nucleotide sequence homology to one or more of the sequences of (i); or
(iii) a functional fragment of (i) or (ii).

126. A nucleic acid construct according to claim 115, wherein the heterologous intron of (c) comprises a sequence selected from the rat insulin gene intron A sequence, chicken keratin gene intron A sequence, chicken cardiac actin gene intron A sequence, a functional fragment of any thereof or a functional variant of any of the preceding.

127. A nucleic acid construct according to claim 126, wherein the rat insulin gene intron A sequence comprises:
(i) the nucleotide sequence of SEQ ID NO:3, the nucleotide sequence of nucleotides 1725 to 1857 of SEQ ID No. 54, nucleotides 1545 to 1677 of SEQ ID No: 61, nucleotides 2770 to 2902 of SEQ ID No: 61, nucleotides 1824 to 1956 of SEQ ID No:62 and/or nucleotides 3446 to 3578 of SEQ ID No:62;
(ii) a functional variant of (i) which has at least 80% nucleotide sequence homology to one or more of the sequences of (i); or
(iii) a functional fragment of (i) or (ii).

128. A nucleic acid construct according to claim 115, wherein the chimeric promoter sequence comprises:
the nucleotide sequence of SEQ ID No. 4 or the nucleotide sequence of nucleotides 903 to 1857 of SEQ ID No. 54;
(ii) a functional variant of (i) which has at least 80% nucleotide sequence homology to (i); or
(iii) a functional fragment of (i) or (ii).

129. A nucleic acid construct according to claim 115, which comprises:
(i) the sequence of the vector pPJV7563 provided as SEQ ID No:14, or
(ii) a sequence with at least 60% sequence identity to the sequence of (i);
and the sequence encoding the said antigen, fragment or variant is provided in the said sequence (i) or (ii) so that it is operably linked to the chimeric promoter.

130. A nucleic acid construct according to claim 115, which comprises the sequence of any one of the vectors:
i) pPJV1671 provided as SEQ ID No: 54 or a sequence with at least 60% sequence identity thereto, and
ii) pPML7789 provided as SEQ ID No: 59 or a sequence with at least 60% sequence identity thereto.

131. A nucleic acid construct according to claim 119, which comprises the sequence of any one of the vectors:
i) pPJV2012 provided as SEQ ID No: 61 or a sequence with at least 60% sequence identity thereto, and
ii) pPJV7788 provided as SEQ ID NO: 62 or a sequence with at least 60% sequence identity thereto.

132. A nucleic acid construct according to claim 115, which further comprises a polyadenylation sequence.

133. A nucleic acid construct according to claim 132, wherein the polyadenylation sequence is a polyadenylation sequence of a gene selected from rabbit beta-globin gene, human papilloma virus (HPV) early or late genes, the HSV-2gB gene, a simian CMV immediate early gene and HSVgD late gene.

134. A nucleic acid construct according to claim 133, wherein the polyadenylation sequence is selected from:
(i) the nucleotide sequence of SEQ ID NO:10, SEQ ID NO:11, SEQ ID NO:12, SEQ ID NO:13, nucleotides 4243 to 4373 of SEQ ID No:54, nucleotides 906 to 1038 of SEQ ID No:61, nucleotides 4375 to 4050 of SEQ ID NO:61, or nucleotides 2463 to 2593 of SEQ ID NO:62;
(ii) a functional variant of a nucleotide sequence of (i) which has at least 80% nucleotide sequence homology to said sequence; or
(iii) a functional fragment of a nucleotide sequence of (i) or (ii).

135. A nucleic acid construct according to claim 119 which comprises the sequence of SEQ ID NO:14.

136. A nucleic acid construct according to claim 115, which further comprises a nucleotide sequence encoding a signal peptide which is operably linked to the sequence encoding the antigen, exotoxin subunit, fragment or variant.

137. A nucleic acid construct according to claim 136, wherein the signal peptide is selected from the human tissue plasminogen activator signal peptide (hTPAsp), aprotinin signal peptide, tobacco extensin signal peptide and chicken lysozyme signal peptide.

138. A nucleic acid construct according to claim 115, which is a plasmid.

139. Carrier particles coated with one or more nucleic acid constructs according to claim 115.

140. Carrier particles according to claim 139, which are gold particles.

141. A dosage receptacle for a particle mediated delivery device comprising the carrier particles of claim 139.

142. A particle mediated delivery device loaded with the carrier particles of claim 139.

143. A particle mediated delivery device according to claim 142, which is a needleless syringe.

144. A population of nucleic acid constructs wherein the population comprises at least two different constructs according to claim 115.

145. A pharmaceutical composition comprising one or more nucleic acid constructs according to claim 115 and a pharmaceutically acceptable carrier or excipient.

146. A vaccine composition comprising a nucleic acid construct according to claim 115.

147. A vaccine composition according to claim 146, which is a multivalent vaccine comprising at least two different constructs which encode different influenza antigens, immunogenic fragments thereof or immunogenic variants of either thereof.

148. A vaccine composition according to claim 147, which is a trivalent, tetravalent or pentavalent influenza vaccine.

149. A method for preventing influenza comprising administering to a subject one or more nucleic acid constructs of claim 115.

150. The method according to claim 149, wherein the nucleic acid construct is delivered by injection, transdermal particle delivery, inhalation, topically, orally, intranasally or transmucosally.

151. The method according to claim 150, wherein the nucleic acid construct is delivered by needleless injection.

152. An in vitro method of obtaining expression in mammalian cells of an influenza polypeptide of interest, which method comprises transferring into said cells one or more nucleic acid constructs of claim 115.

153. A method for preventing influenza comprising administering to a subject the carrier particles of claim 139.

154. The method according to claim 153, wherein the carrier particles are delivered by injection, transdermal particle delivery, inhalation, topically, orally, intranasally or transmucosally.

155. The method according to claim 154, wherein the carrier particles are delivered by needleless injection.

156. An in vitro method of obtaining expression in mammalian cells of an influenza polypeptide of interest, which method comprises transferring into said cells the carrier particles of claim 139.


SEQUENCE LISTING (70 sequences)

SEQ ID NO: 1 - DNA, 685 bp, Human cytomegalovirus
aatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaacc

SEQ ID NO: 2 - DNA, 131 bp, Human cytomegalovirus
gtcagatcgcctggagacgccatccacgctgttttgacctccatagaagacaccgggaccgatccagcctccgcggccgggaacggtgcattggaacgcggattccccgtgccaagagtgactcaccgtcc

SEQ ID NO: 3 - DNA, 135 bp, Rattus rattus
atcagcaagcaggtatgtactctccagggtgggcctggcttccccagtcaagactccagggatttgagggacgctgtgggctcttctcttacatgtaccttttgctagcctcaaccctgactatcttccaggtca

SEQ ID NO: 4 - DNA, 955 bp, Artificial Sequence
  Description of Artificial Sequence Synthetic Chimeric promotor sequence
aatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgactcaccgtccggatctcagcaagcaggtatgtactctccagggtgggcctggcttccccagtcaagactccagggatttgagggacgctgtgggctcttctcttacatgtaccttttgcttgcctcaaccctgactatcttccaggtca

SEQ ID NO: 5 - DNA, 121 bp, Hepatitis B virus
cagagtcaggggtctgtattttcctgctggtggctccagttcaggaacagtaaaccctgctccgaatattgcctctcacatctcgtcaatctccgcgaggactggggaccctgtgacgaac

SEQ ID NO: 6 - DNA, 57 bp, Herpes simplex virus
ataagctgcattgcgaaccactagtcgccgtttttcgtgtgcatcgcgtatcacggc

SEQ ID NO: 7 - DNA, 48 bp, Hepatitis B virus
ctttgtactaggaggctgtaggcataaattggtctgttcaccagcacc

SEQ ID NO: 8 - DNA, 533 bp, Hepatitis B virus
taacaaaacaaaaagatggggttattccctaaacttcatgggttacgtaattggaagttgggggacattgccacaagatcatattgtacaaaagatcaaacactgttttagaaaacttcctgtaaacaggcctattgattggaaagtatgtcaaaggattgtgggtcttttgggctttgctgctccatttacacaatgtggatatcctgccttaatgcctttgtatgcatgtatacaagctaaacaggctttcactttctcgccaacttacaaggcctttctaagtaaacagtacatgaacctttaccccgttgctcggcaacggcctggtctgtgccaagtgtttgctgacgcaacccccactggctggggcttggccataggccatcagcgcatgcgtggaacctttgtggctcctctgccgatccatactgcggaactcctagccgcttgttttgctcgcagccggtctggagcaaagctcataggaactgacaattctgtcgtcctctcgcggaaatatacatcgtttc

SEQ ID NO: 9 - DNA, 158 bp, Simian cytomegalovirus
gtcagacagacagacagttatatgggctggtccctataactctgccattgtaaccccatatagccagacagttagcattgcatctattgatgatgtactaatgtattgtaaccccccctatgccattgtctaactgtactaatgtatgatattatacc

SEQ ID NO: 10 - DNA, 131 bp, Oryctolagus cuniculus
gatctttttccctctgccaaaaattatggggacatcatgaagccccttgagcatctgacttctggctaataaaggaaatttattttcattgcaatagtgtgttggaattttttgtgtctctcactcggaag

SEQ ID NO: 11 - DNA, 204 bp, Simian cytomegalovirus
atatatactctatgttatactctatgatatacaatatatactcatgaacactatgtacttggtgtatgactcattattgtctgggacttggttgggacttggttggttgggaagaatgttgtgcctgtacttgtgctgtgctgtggatctcaataaatgtgactatgttcaaaacactaagtgcccccgtgtcttctttaacta

SEQ ID NO: 12 - DNA, 163 bp, Herpes simplex virus 2
gaagacgagctctaagggaggggaggggagctgggcttgtgtataaataaaaagacaccgatgttcaaaaatacacatgacttctggtattgttttgccttggtttttatttgggggggggggggcgtgtgactagaaaaacaaatgcagacatgtgctaacg

SEQ ID NO: 13 - DNA, 191 bp, Human papillomavirus
  Type 16
aattgttacatataattgttgtataccataacttactattttttcttttttattttcatatataatttttttttttgtttgtttgtttgttttttaataaactgttattacttaacaatgcgacacaaacgttctgcaaaacgcacaaaacgtgcatcggctacccaactttataaaacatgcaaacaggc

SEQ ID NO: 14 - DNA, 3759 bp, Artificial Sequence
  Description of Artificial Sequence Synthetic pJV expression vector
ggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacaggtcgacaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgactcaccgtccggatctcagcaagcaggtatgtactctccagggtgggcctggcttccccagtcaagactccagggatttgagggacgctgtgggctcttctcttacatgtaccttttgcttgcctcaaccctgactatcttccaggtcaggatcccagagtcaggggtctgtattttcctgctggtggctccagttcaggaacagtaaaccctgctccgaatattgcctctcacatctcgtcaatctccgcgaggactggggaccctgtgacgaacatggctagcgggcccagatctgggccctaacaaaacaaaaagatggggttattccctaaacttcatgggttacgtaattggaagttgggggacattgccacaagatcatattgtacaaaagatcaaacactgttttagaaaacttcctgtaaacaggcctattgattggaaagtatgtcaaaggattgtgggtcttttgggctttgctgctccatttacacaatgtggatatcctgccttaatgcctttgtatgcatgtatacaagctaaacaggctttcactttctcgccaacttacaaggcctttctaagtaaacagtacatgaacctttaccccgttgctcggcaacggcctggtctgtgccaagtgtttgctgacgcaacccccactggctggggcttggccataggccatcagcgcatgcgtggaacctttgtggctcctctgccgatccatactgcggaactcctagccgcttgttttgctcgcagccggtctggagcaaagctcataggaactgacaattctgtcgtcctctcgcggaaatatacatcgtttcgatctacgtatgatctttttccctctgccaaaaattatggggacatcatgaagccccttgagcatctgacttctggctaataaaggaaatttattttcattgcaatagtgtgttggaattttttgtgtctctcactcggaaggaattctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactc

SEQ ID NO: 15 - DNA, 42 bp, Artificial Sequence
  Description of Artificial Sequence Synthetic primer
ggaggatccggacggtgagtcactcttggcacggggaatccg

SEQ ID NO: 16 - DNA, 21 bp, Artificial Sequence
  Description of Artificial Sequence Synthetic primer
ggtgaatatggctcataacac

SEQ ID NO: 17 - DNA, 23 bp, Artificial Sequence
  Description of Artificial Sequence Synthetic primer
ccgccgaacatggagaacatcgc

SEQ ID NO: 18 - DNA, 33 bp, Artificial Sequence
  Description of Artificial Sequence Synthetic primer
cacagatcttttgttagggtttaaatgtatacc

SEQ ID NO: 19 - DNA, 29 bp, Artificial Sequence
  Description of Artificial Sequence Synthetic primer
ggaggatcctgacctggaagatagtcacc

SEQ ID NO: 20 - DNA, 26 bp, Artificial Sequence
  Description of Artificial Sequence Synthetic primer
ggaggatccatcagcaagcaggtatg

SEQ ID NO: 21 - DNA, 33 bp, Artificial Sequence
  Description of Artificial Sequence Synthetic primer
ggagctagcgggcgtttgacctccggcgtcggg

SEQ ID NO: 22 - DNA, 37 bp, Artificial Sequence
  Description of Artificial Sequence Synthetic primer
ggagaattcagatctcctctagtaaaacaatggctgg

SEQ ID NO: 23 - DNA, 25 bp, Artificial Sequence
  Description of Artificial Sequence Synthetic primer
ggagctagccttctaaccgaggtcg

SEQ ID NO: 24 - DNA, 30 bp, Artificial Sequence
  Description of Artificial Sequence Synthetic primer
ggaagatctccttactccagctctatgctg

SEQ ID NO: 25 - DNA, 27 bp, Artificial Sequence
  Description of Artificial Sequence Synthetic primer
ggcgaattccttccgagtgagagacac

SEQ ID NO: 26 - DNA, 43 bp, Artificial Sequence
  Description of Artificial Sequence Synthetic primer
ggagtatacatttaaagggccctaacaaaacaaaaagatgggg

SEQ ID NO: 27 - DNA, 31 bp, Artificial Sequence
  Description of Artificial Sequence Synthetic primer
ggagctagctcgtttactttgaccaagaacg

SEQ ID NO: 28 - DNA, 36 bp, Artificial Sequence
  Description of Artificial Sequence Synthetic primer
ggaagatctccttatttttgacaccagaccaactgg

SEQ ID NO: 29 - DNA, 29 bp, Artificial Sequence
  Description of Artificial Sequence Synthetic primer
ggagtcgacctgtctgcttacataaacag

SEQ ID NO: 30 - DNA, 26 bp, Artificial Sequence
  Description of Artificial Sequence Synthetic primer
cgtaatgctctgccagtgttacaacc

SEQ ID NO: 31 - DNA, 20 bp, Artificial Sequence
  Description of Artificial Sequence Synthetic primer
gaaagatctcagcaagcagg

SEQ ID NO: 32 - DNA, 47 bp, Artificial Sequence
  Description of Artificial Sequence Synthetic primer
ggaggatcctgacctggaagatagtcagggttgaggcaagcaaaagg

SEQ ID NO: 33 - DNA, 12 bp, Artificial Sequence
  Description of Artificial Sequence Synthetic primer
ctagcgggccca

SEQ ID NO: 34 - DNA, 12 bp, Artificial Sequence
  Description of Artificial Sequence Synthetic primer
gatctgggcccg

SEQ ID NO: 35 - DNA, 28 bp, Artificial Sequence
  Description of Artificial Sequence Synthetic primer
ggagctagcatcatcccagttgaggagg

SEQ ID NO: 36 - DNA, 28 bp, Artificial Sequence
  Description of Artificial Sequence Synthetic primer
ggtagatctcctcatgtctgctcgaagc

SEQ ID NO: 37 - DNA, 29 bp, Artificial Sequence
  Description of Artificial Sequence Synthetic primer
ccaagctagcgacaaaactcacacatgcc

SEQ ID NO: 38 - DNA, 45 bp, Artificial Sequence
  Description of Artificial Sequence Synthetic primer
ggaagatctcgtttacccctgtcatttacccggagacagggagag

SEQ ID NO: 39 - DNA, 57 bp, Artificial Sequence
  Description of Artificial Sequence Synthetic oligonucleotide
aagatgtccagactctgtctctccgtggccctcctcgtgctcctcgggacactcgcc

SEQ ID NO: 40 - DNA, 24 bp, Artificial Sequence
  Description of Artificial Sequence Synthetic primer
ggaactagtaagatgtccagactc

SEQ ID NO: 41 - DNA, 25 bp, Artificial Sequence
  Description of Artificial Sequence Synthetic primer
ggaagctagcggcgagtgtcccgag

SEQ ID NO: 42 - DNA, 75 bp, Artificial Sequence
  Description of Artificial Sequence Synthetic oligonucleotide
ggaaagatggccagcctctttgccacatttctcgtggtgctcgtgagcctcagcctcgccagcgaaagcagcgcc

SEQ ID NO: 43 - DNA, 24 bp, Artificial Sequence
  Description of Artificial Sequence Synthetic primer
ggaactagtggaaagatggccagc

SEQ ID NO: 44 - DNA, 26 bp, Artificial Sequence
  Description of Artificial Sequence Synthetic primer
ggaagctagcggcgctgctttcgctg

SEQ ID NO: 45 - DNA, 51 bp, Artificial Sequence
  Description of Artificial Sequence Synthetic oligonucleotide
aggtctttgctaatcttggtgctttgcttcctgcccctggctgctctgggg

SEQ ID NO: 46 - DNA, 24 bp, Artificial Sequence
  Description of Artificial Sequence Synthetic primer
ggaactagtaggtctttgctaatc

SEQ ID NO: 47 - DNA, 25 bp, Artificial Sequence
  Description of Artificial Sequence Synthetic primer
ggaagctagcccccagagcagccag

SEQ ID NO: 48 - DNA, 31 bp, Artificial Sequence; identical to SEQ ID NO: 27
  Description of Artificial Sequence Synthetic primer

SEQ ID NO: 49 - DNA, 25 bp, Artificial Sequence
  Description of Artificial Sequence Synthetic primer
ggaagatctccggtgagtggtgctg

SEQ ID NO: 50 - DNA, 32 bp, Artificial Sequence
  Description of Artificial Sequence Synthetic primer
gcaggatccagtagacctggagagaggacaag

SEQ ID NO: 51 - DNA, 29 bp, Artificial Sequence
  Description of Artificial Sequence Synthetic primer
ggaagatctacaaggtgagctgctgtggc

SEQ ID NO: 52 - DNA, 490 bp, Pseudo rabies virus
tggccgcagagcgggccgggcatgcaaatcagaggcgcgcgggagacgcctccgcgcgcccattggcccgggcgagccgagatggccgccgcgggggccggacatgcaaagtagacgcgagaggaagtagggagagaaatcccattggccgtcgaggggccaagatggcgccctcggggccggacatgcaaagtagacgcgagaggaagtgggcgagagaaatcccattggccgtcgatggggcaagatggccgccgcgggggccgggcatgcaaatggtcctcgcgaggaagttcctcgcgaaatcccattggccggcggccgccatcttgggccgggcatgcaaagcagacggcagaggaagcgggcgagaaaaatcccattggccggccgtcggggaagtccgcggcgaaaatcggccattggtccgcttacctgggggcgggctctcctcggggcgcttataagcgcggtctccatcgtagcactt

SEQ ID NO: 53 - DNA, 495 bp, Rous sarcoma virus
ctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagttccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatt

SEQ ID NO: 54 - DNA, 5446 bp, Artificial Sequence
  Description of Artificial Sequence Synthetic polynucleotide
ggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcaaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacaggtcgacaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgactcaccgtccggatctcagcaagcaggtatgtactctccagggtgggcctggcttccccagtcaagactccagggatttgagggacgctgtgggctcttctcttacatgtaccttttgcttgcctcaaccctgactatcttccaggtcaggatcccagagtcaggggtctgtattttcctgctggtggctccagttcaggaacagtaaaccctgctccgaatattgcctctcacatctcgtcaatctccgcgaggactggggaccctgtgacgaacatggctagcaagactatcattgctttgagctacattttatgtctggttttcgctcaaaaacttcccggaaatgacaacagcacggcaacgctgtgcctggggcaccatgcagtgtcaaacggaacgctagtgaaaacaatcacgaatgaccaaattgaagtgactaatgctactgagctggttcagagttcctcaacaggtagaatatgcgacagtcctcaccaaatccttgatggagaaaactgcacactaatagatgctctattgggagaccctcattgtgatggcttccaaaataaggaatgggacctttttgttgaacgcagcaaagcctacagcaactgttacccttatgatgtgccggattatgcctcccttaggtcactagttgcctcatccggcacactggagtttaacaatgaaagcttcaattggactggagtcgctcagaatggaacaagctctgcttgcaaaaggagatctaataaaagtttctttagtagattgaattggttgcaccaattaaaatacaaatatccagcactgaacgtgactatgccaaacaatgaaaaatttgacaaattgtacatttggggggttctccacccgagtacggacagtgaccaaatcagcctatatgctcaagcatcagggagagtcacagtctctaccaaaagaagccaacaaactgtaatcccgaatatcggatctagaccctgggtaaggggtgtctccagcagaataagcatctattggacaatagtaaaaccgggagacatacttttgattaacagcacagggaatctaattgctcctcggggttacttcaaaatacgaagtgggaaaagctcaataatgaggtcagatgcacccattggcaaatgcaattctgaatgcatcactccaaatggaagcattcccaatgacaaaccatttcaaaatgtaaacaggatcacatatggggcctgtcccagatatgttaagcaaaacactctgaaattggcaacagggatgcggaatgtaccagagaaacaaactagaggcatattcggcgcaatcgcgggtttcatagaaaatggttgggagggaatggtggacggttggtacggtttcaggcatcaaaattctgagggcacaggacaagcagcagatcttaaaagcactcaagcagcaatcaaccaaatcaacgggaaactgaataggttaatcgagaaaacgaacgagaaattccatcaaattgaaaaagaattctcagaagtagaagggagaattcaggacctcgagaaatatgttgaggacactaaaatagatctctggtcgtacaacgcggagcttcttgttgccctggagaaccaacatacaattgatctaactgactcagaaatgaacaaactgtttgaaagaacaaagaagcaactgagggaaaatgctgaggatatgggcaatggttgtttcaaaatataccacaaatgtgacaatgcctgcatagggtcaatcagaaatggaacttatgaccatgatgtatacagagacgaagcattaaacaaccggttccagatcaaaggtgttgagctgaagtcaggatacaaagattggatcctatggatttcctttgccatatcatgctttttgctttgtgttgttttgctggggttcatcatgtgggcctgccaaaaaggcaacattaggtgcaacatttgcatttgaggggccctaacaaaacaaaaagatggggttattccctaaacttcatgggttacgtaattggaagttgggggacattgccacaagatcatattgtacaaaagatcaaacactgttttagaaaacttcctgtaaacaggcctattgattggaaagtatgtcaaaggattgtgggtcttttgggctttgctgctccatttacacaatgtggatatcctgccttaatgcctttgtatgcatgtatacaagctaaacaggctttcactttctcgccaacttacaaggcctttctaagtaaacagtacatgaacctttaccccgttgctcggcaacggcctggtctgtgccaagtgtttgctgacgcaacccccactggctggggcttggccataggccatcagcgcatgcgtggaacctttgtggctcctctgccgatccatactgcggaactcctagccgcttgttttgctcgcagccggtctggagcaaagctcataggaactgacaattctgtcgtcctctcgcggaaatatacatcgtttcgatctacgtatgatctttttccctctgccaaaaattatggggacatcatgaagccccttgagcatctgacttctggctaataaaggaaatttattttcattgcaatagtgtgttggaattttttgtgtctctcactcggaaggaattctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactc

SEQ ID NO: 55 - protein, 568 aa, Artificial Sequence
  Description of Artificial Sequence Synthetic polypeptide
MASKTIIALSYILCLVFAQKLPGNDNSTATLCLGHHAVSNGTLVKTITNDQIEVTNATELVQSSSTGRICDSPHQILDGENCTLIDALLGDPHCDGFQNKEWDLFVERSKAYSNCYPYDVPDYASLRSLVASSGTLEFNNESFNWTGVAQNGTSSACKRRSNKSFFSRLNWLHQLKYKYPALNVTMPNNEKFDKLYIWGVLHPSTDSDQISLYAQASGRVTVSTKRSQQTVIPNIGSRPWVRGVSSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQINGKLNRLIEKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 56 - protein, 63 aa, Artificial Sequence
  Description of Artificial Sequence Synthetic N terminal peptide of H3 Panama HA influenza antigen
MKTIIALSYILCLVFAQKLPGNDNSTATLCLGHHAVSNGTLVKTITNDQIEVTNATELVQSSS

SEQ ID NO: 57 - protein, 65 aa, Artificial Sequence
  Description of Artificial Sequence Synthetic N terminal sequence of H3 Panama HA antigen encoded by pPJV1671
MASKTIIALSYILCLVFAQKLPGNDNSTATLCLGHHAVSNGTLVKTITNDQIEVTNATELVQSSS

SEQ ID NO: 58 - protein, 62 aa, Artificial Sequence
  Description of Artificial Sequence Synthetic consensus of natural and pPJV1671 N terminal H3 Panama HA antigens
KTIIALSYILCLVFAQKLPGNDNSTATLCLGHHAVSNGTLVKTITNDQIEVTNATELVQSSS

SEQ ID NO: 59 - DNA, 5453 bp, Artificial Sequence
  Description of Artificial Sequence Synthetic polynucleotide
ggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacaggtcgacaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgactcaccgtccggatctcagcaagcaggtatgtactctccagggtgggcctggcttccccagtcaagactccagggatttgagggacgctgtgggctcttctcttacatgtaccttttgcttgcctcaaccctgactatcttccaggtcaggatcccagagtcaggggtctgtattttcctgctggtggctccagttcaggaacagtaaaccctgctccgaatattgcctctcacatctcgtcaatctccgcgaggactggggaccctgtgacgaacatggctagcgagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagacacacaatgggaagctctgcgatctagatggagtgaagcctctaattttgagagattgtagtgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattgggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaaaccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccatcggggaatgccccaaatatgtgaaatcaaacagattagtccttgcgactgggctcagaaatagccctcaaagagagagaagaagaaaaaagagaggattatttggagctatagcaggttttatagagggaggatggcagggaatggtagatggttggtatgggtaccaccatagcaacgagcaggggagtgggtacgctgcagacaaagaatccactcaaaaggcaatagatggagtcaccaataaggtcaactcgattattgacaaaatgaacactcagtttgaggccgttggaagggaatttaacaacttagaaaggagaatagagaatttaaacaagaagatggaagacgggttcctagatgtctggacttataatgctgaacttctagttctcatggaaaacgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaacggaacgtatgactacccgcagtattcagaagaagcaagactaaaaagagaggaaataagtggagtaaaattggaatcaataggaatttaccaaatattgtcaatttattctacagtggcgagctccctagcactggcaatcatggtagctggtctatccttatggatgtgctccaatgggtcgttacaatgcagaatttgcatttaaatgggccctaacaaaacaaaaagatggggttattccctaaacttcatgggttacgtaattggaagttgggggacattgccacaagatcatattgtacaaaagatcaaacactgttttagaaaacttcctgtaaacaggcctattgattggaaagtatgtcaaaggattgtgggtcttttgggctttgctgctccatttacacaatgtggatatcctgccttaatgcctttgtatgcatgtatacaagctaaacaggctttcactttctcgccaacttacaaggcctttctaagtaaacagtacatgaacctttaccccgttgctcggcaacggcctggtctgtgccaagtgtttgctgacgcaacccccactggctggggcttggccataggccatcagcgcatgcgtggaacctttgtggctcctctgccgatccatactgcggaactcctagccgcttgttttgctcgcagccggtctggagcaaagctcataggaactgacaattctgtcgtcctctcgcggaaatatacatcgtttcgatctacgtatgatctttttccctctgccaaaaattatggggacatcatgaagccccttgagcatctgacttctggctaataaaggaaatttattttcattgcaatagtgtgttggaattttttgtgtctctcactcggaaggaattctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactc

SEQ ID NO: 60 - protein, 570 aa, Artificial Sequence
  Description of Artificial Sequence Synthetic polypeptide
MASEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 61 - DNA, 5578 bp, Artificial Sequence
  Description of Artificial Sequence Synthetic PJV2012 construct containing LTA and TLB coding sequences
ggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcaaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacaggtcgacaattccttccgagtgagagacacaaaaaattccaacacactattgcaatgaaaataaatttcctttattagccagaagtcagatgctcaaggggcttcatgatgtccccataatttttggcagagggaaaaagatccctagttttccatactgattgccgcaattgaattgggggttttattattccatacacataatttatcaattttggtctcggtcagatatgtgattcttaatgtgtccttcatcctttcaatggcttttttttgggagtctatatgttgactgcccgggacttcgacctgaaatgttgcgccgctcttaaatgtaatgataaccatttctcttttgcctgccatcgattccgtatatgatagtatcttgtcatttatcgtatatatttgtgtgttgcgatattccgaacatagttctgtaatagactggggagcgctagcccccagagcagccaggggcaggaagcaaagcaccaagattagcaaagacctactagccatgttcgtcacagggtccccagtcctcgcggagattgacgagatgtgagaggcaatattcggagcagggtttactgttcctgaactggagccaccagcaggaaaatacagacccctgactctgggatcctgacctggaagatagtcagggttgaggcaagcaaaaggtacatgtaagagaagagcccacagcgtccctcaaatccctggagtcttgactggggaagccaggcccaccctggagagtacatacctgcttgctgagatccggacggtgagtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaacggggcggggttattacgacattttggaaagtcccgttgattttggtgctcgacctgcaggtcgacaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgactcaccgtccggatctcagcaagcaggtatgtactctccagggtgggcctggcttccccagtcaagactccagggatttgagggacgctgtgggctcttctcttacatgtaccttttgcttgcctcaaccctgactatcttccaggtcaggatcccagagtcaggggtctgtattttcctgctggtggctccagttcaggaacagtaaaccctgctccgaatattgcctctcacatctcgtcaatctccgcgaggactggggaccctgtgacgaacatggctagtaggtctttgctaatcttggtgctttgcttcctgcccctggctgctctgggggctagcaatggcgacaaattataccgtgctgactctagacccccagatgaaataaaacgttccggaggtcttatgcccagagggcataatgagtacttcgatagaggaactcaaatgaatattaatctttatgatcacgcgagaggaacacaaaccggctttgtcagatatgatgacggatatgtttccacttctcttagtttgagaagtgctcacttagcaggacagtctatattatcaggatattccacttactatatatatgttatagcgacagcaccaaatatgtttaatgttaatgatgtattaggcgtatacagccctcacccatatgaacaggaggtttctgcgttaggtggaataccatattctcagatatatggatggtatcgtgttaattttggtgtgattgatgaacgattacatcgtaacagggaatatagagaccggtattacagaaatctgaatatagctccggcagaggatggttacagattagcaggtttcccaccggatcaccaagcttggagagaagaaccctggattcatcatgcaccacaaggttgtggaaattcatcaagaacaattacaggtgatacttgtaatgaggagacccagaatctgagcacaatatatctcaggaaatatcaatcaaaagttaagaggcagatattttcagactatcagtcagaggttgacatatataacagaattcgggatgaattatgaggatctgggccctaacaaaacaaaaagatggggttattccctaaacttcatgggttacgtaattggaagttgggggacattgccacaagatcatattgtacaaaagatcaaacactgttttagaaaacttcctgtaaacaggcctattgattggaaagtatgtcaaaggattgtgggtcttttgggctttgctgctccatttacacaatgtggatatcctgccttaatgcctttgtatgcatgtatacaagctaaacaggctttcactttctcgccaacttacaaggcctttctaagtaaacagtacatgaacctttaccccgttgctcggcaacggcctggtctgtgccaagtgtttgctgacgcaacccccactggctggggcttggccataggccatcagcgcatgcgtggaacctttgtggctcctctgccgatccatactgcggaactcctagccgcttgttttgctcgcagccggtctggagcaaagctcataggaactgacaattctgtcgtcctctcgcggaaatatacatcgtttcgatctacgtatgatctttttccctctgccaaaaattatggggacatcatgaagccccttgagcatctgacttctggctaataaaggaaatttattttcattgcaatagtgtgttggaattttttgtgtctctcactcggaaggaattctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactc

SEQ ID NO: 62 - DNA, 6254 bp, Artificial Sequence
  Description of Artificial Sequence Synthetic PJV7788 construct
ggcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccggcatgcctgcaggtcgacaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgactcaccgtccggatctcagcaagcaggtatgtactctccagggtgggcctggcttccccagtcaagactccagggatttgagggacgctgtgggctcttctcttacatgtaccttttgcttgcctcaaccctgactatcttccaggtcaggatcccagagtcaggggtctgtattttcctgctggtggctccagttcaggaacagtaaaccctgctccgaatattgcctctcacatctcgtcaatctccgcgaggactggggaccctgtgacgaacatggctagtaggtctttgctaatcttggtgctttgcttcctgcccctggctgctctgggggctagcgctccccagtctattacagaactatgttcggaatatcgcaacacacaaatatatacgataaatgacaagatactatcatatacggaatcgatggcaggcaaaagagaaatggttatcattacatttaagagcggcgcaacatttcaggtcgaagtcccgggcagtcaacatatagactcccaaaaaaaagccattgaaaggatgaaggacacattaagaatcacatatctgaccgagaccaaaattgataaattatgtgtatggaataataaaacccccaattcaattgcggcaatcagtatggaaaactagggatctttttccctctgccaaaaattatggggacatcatgaagccccttgagcatctgacttctggctaataaaggaaatttattttcattgcaatagtgtgttggaattttttgtgtctctcactcggaaggaattgagggccggccctctgcaggtcgacaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgactcaccgtccggatctcagcaagcaggtatgtactctccagggtgggcctggcttccccagtcaagactccagggatttgagggacgctgtgggctcttctcttacatgtaccttttgcttgcctcaaccctgactatcttccaggtcaggatcccagagtcaggggtctgtattttcctgctggtggctccagttcaggaacagtaaaccctgctccgaatattgcctctcacatctcgtcaatctccgcgaggactggggaccctgtgacgaacatggctagtaggtctttgctaatcttggtgctttgcttcctgcccctggctgctctgggggctagcaatggcgacaaattataccgtgctgactctagacccccagatgaaataaaacgttccggaggtcttatgcccagagggcataatgagtacttcgatagaggaactcaaatgaatattaatctttatgatcacgcgagaggaacacaaaccggctttgtcagatatgatgacggatatgtttccacttctcttagtttgagaagtgctcacttagcaggacagtctatattatcaggatattccacttactatatatatgttatagcgacagcaccaaatatgtttaatgttaatgatgtattaggcgtatacagccctcacccatatgaacaggaggtttctgcgttaggtggaataccatattctcagatatatggatggtatcgtgttaattttggtgtgattgatgaacgattacatcgtaacagggaatatagagaccggtattacagaaatctgaatatagctccggcagaggatggttacagattagcaggtttcccaccggatcaccaagcttggagagaagaaccctggattcatcatgcaccacaaggttgtggaaattcatcaagaacaattacaggtgatacttgtaatgaggagacccagaatctgagcacaatatatctcaggaaatatcaatcaaaagttaagaggcagatattttcagactatcagtcagaggttgacatatataacagaattcgggatgaattatgaggatctgggccctaacaaaacaaaaagatggggttattccctaaacttcatgggttacgtaattggaagttgggggacattgccacaagatcatattgtacaaaagatcaaacactgttttagaaaacttcctgtaaacaggcctattgattggaaagtatgtcaaaggattgtgggtcttttgggctttgctgctccatttacacaatgtggatatcctgccttaatgcctttgtatgcatgtatacaagctaaacaggctttcactttctcgccaacttacaaggcctttctaagtaaacagtacatgaacctttaccccgttgctcggcaacggcctggtctgtgccaagtgtttgctgacgcaacccccactggctggggcttggccataggccatcagcgcatgcgtggaacctttgtggctcctctgccgatccatactgcggaactcctagccgcttgttttgctcgcagccggtctggagcaaagctcataggaactgacaattctgtcgtcctctcgcggaaatatacatcgtttcgatctacgtatgatctttttccctctgccaaaaattatggggacatcatgaagccccttgagcatctgacttctggctaataaaggaaatttattttcattgcaatagtgtgttggaattttttgtgtctctcactcggaaggaattctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactc

SEQ ID NO: 63 - DNA, 18 bp, Artificial Sequence
  Description of Artificial Sequence Synthetic oligonucleotide
gccactctcttccgacac

SEQ ID NO: 64 - DNA, 19 bp, Artificial Sequence
  Description of Artificial Sequence Synthetic oligonucleotide
caagaacatcacacggaac

SEQ ID NO: 65 - protein, 512 aa, Herpes Simplex virus 2
MATDIDMLIDLGLDLSDSELEEDALERDEEGRRDDPESDSSGECSSSDEDMEDPCGDGGAEAIDAAIPKGPPARPEDAGTPEASTPRPAARRGADDPPPATTGVWSRLGTRRSASPREPHGGKVARIQPPSTKAPHPRGGRRGRRRGRGRYGPGGADSTPNPRRRVSRNAHNQGGRHPASARTDGPGATHGEARRGGEQLDVSGGPRPRGTRQAPPPLMALSLTPPHADGRAPVPERKAPSADTIDPAVRAVLRSISERAAVERISESFGRSALVMQDPFGGMPFPAANSPWAPVLATQAGGFDAETRRVSWETLVAHGPSLYRTFAANPRAASTAKAMRDCVLRQENLIEALASADETLAWCKMCIHHNLPLRPQDPIIGTAAAVLENLATRLRPFLQCYLKARGLCGLDDLCSRRRLSDIKDIASFVLVILARLANRVERGVSEIDYTTVGVGAGETMHFYIPGACMAGLIEILDTHRQECSSRVCELTASHTIAPLYVHGKYFYCNSLF

SEQ ID NO: 66 - protein, 18 aa, Herpes Simplex virus 2
RVSWETLVAHGPSLYRTF

SEQ ID NO: 67 - protein, 18 aa, Herpes Simplex virus 2
VAHGPSLYRTFAANPRAA

SEQ ID NO: 68 - protein, 9 aa, Artificial Sequence
  Description of Artificial Sequence Synthetic peptide
HGPSLYRTF

SEQ ID NO: 69 - protein, 11 aa, Herpes Simplex virus 2
LYRTFAANPRA

SEQ ID NO: 70 - protein, 11 aa, Herpes Simplex virus 1
LYRTFAGNPRA